(19) United States
(12) Patent Application Publication
(10) Pub. No.: US 2005/0137172 A1
(43) Pub. Date: Jun. 23, 2005

(54) Haloacetamide and azide substituted compounds and methods of use thereof

(76) Inventors: James T. Dalton, Upper Arlington, OH (US); Duane D. Miller, Germantown, TN (US); Vipin Nair, Memphis, TN (US); Huiping Xu, Groton, CT (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/963,761

(22) Filed: Oct. 14, 2004

Related U.S. Application Data
(63) Continuation-in-part of application No. 10/684,582, filed on Oct. 15, 2003.

(51) Int. Cl. A61K 31/66; A61K 31/655; A61K 31/4015; A61K 31/277; A61K 31/32; A61K 31/195

(52) U.S. Cl. 514/114; 514/151; 514/425; 514/522; 514/616; 514/493; 514/563; 514/514

(57) ABSTRACT

The present invention relates to a novel class of anti-cancer compounds, which contain a haloacetamide or azide moiety and are, in one embodiment, alkylating agents. These agents, either alone or in a composition, are useful for treating cancer, preventing cancer, delaying the progression of cancer, treating and/or preventing the recurrence of cancer, suppressing, inhibiting or reducing the incidence of cancer, or inducing apoptosis in a cancer cell. Accordingly, the present invention provides a) methods of treating cancer in a subject; b) methods of preventing cancer in a subject; c) methods of delaying the progression of cancer in a subject; d) methods of treating the recurrence of cancer in a subject; e) methods of preventing the recurrence of cancer in a subject; f) methods of suppressing, inhibiting or reducing the incidence of cancer in a subject; and g) methods of inducing apoptosis in a cancer cell; by administering to the subject an anti-cancer compound of the present invention or an analog or metabolite thereof, its N-oxide, ester, pharmaceutically acceptable salt, hydrate, or any combination thereof as described herein.

### CROSS REFERENCE TO RELATED APPLICATIONS

This Application claims the benefit of U.S. Ser. No. 10/684,582, filed Oct. 15, 2003, which is hereby incorporated by reference.

### GOVERNMENT INTEREST STATEMENT

This invention was made in whole or in part with government support under grant number R29 CA068096 awarded by the National Cancer Institute, National Institute of Health, and under grant number R15 HD35329, awarded by the National Institute of Child Health and Human Development, National Institute of Health. The government may have certain rights in the invention.

### FIELD OF THE INVENTION

The present invention relates to a novel class of anti-cancer compounds that contain a haloacetamide or azide moiety. More particularly, the present invention provides a) methods of treating cancer in a subject; b) methods of preventing cancer in a subject; c) methods of delaying the progression of cancer in a subject; d) methods of treating the recurrence of cancer in a subject; e) methods of preventing the recurrence of cancer in a subject; f) methods of suppressing, inhibiting or reducing the incidence of cancer in a subject; and g) methods of inducing apoptosis in a cancer cell, by administering to the subject an anti-cancer compound of the present invention or an analog or metabolite thereof, its N-oxide, ester, pharmaceutically acceptable salt, hydrate, or any combination thereof as described herein.

### BACKGROUND OF THE INVENTION

Cancer is a disorder in which a population of cells has become, in varying degrees, unresponsive to the control mechanisms that normally govern proliferation and differentiation. The leading therapies to date are surgery, radiation and chemotherapy.

Traditionally, chemotherapeutic treatment of cancer has focused on killing cancer cells directly by exposing them to cytotoxic substances. Ideally, cytotoxic agents are specific for cancer and tumor cells while not affecting or having a mild effect on normal cells. Unfortunately, most cytotoxic agents target especially rapidly dividing cells (both tumor and normal) and thus injure both n eoplastic and normal cell populations.

Currently therapeutic agents used in clinical cancer therapy are categorized into six groups: alkylating agents, antibiotic agents, antimetabolic agents, biologic agents, hormonal agents, and plant-derived agents.

Alkylating agents are polyfunctional compounds that have the ability to substitute alkyl groups for hydrogen ions. These compounds react with phosphate, amino, hydroxyl, sulfihydryl, carboxyl, and imidazole groups. Examples of alkylating agents include bischloroethylamines (nitrogen mustards), aziridines, alkyl alkone sulfonates, nitrosoureas, and platinum compounds. Under physiological conditions, these drugs ionize and produce positively charged ions that attach to susceptible nucleic acids and proteins, leading to cell cycle arrest and/or cell death. The alkylating agents are cell cycle phase nonspecific agents because they exert their activity independently of the specific phase of the cell cycle. The nitrogen mustards and alkyl alkone sulfonates are most effective against cells in the G1 or M phase. Nitrosoureas, nitrogen mustards, and aziridines impair progression from the G1 and S phases to the M phase.

Antibiotic agents are a group of drugs that are produced in a manner similar to antibiotics as a modification of natural products. Examples of antibiotic agents include anthracyclines, mitomycin C, bleomycin, dactinomycin, and plicatomycin. These antibiotic agents interfere with cell growth by targeting various cellular components. For example, anthracyclines are generally believed to interfere with the action of DNA topoisomerase II in the regions of transcriptionally active DNA, which leads to DNA strand scissions.

The antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine.

Hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Plant-derived agents are a group of drugs that are derived from plants or modified based on the molecular structure of the agents. Examples of plant derived agents include vinca alkaloids, podophyllotoxins, and taxanes. These plant derived agents generally act as antimitotic agents that bind to tubulin and inhibit mitosis. Podophyllotoxins such as etoposide are believed to interfere with DNA synthesis by interacting with topoisomerase II, leading to DNA strand scission.

Biologic agents are a group of biomolecules that elicit cancer/tumor regression when used alone or in combination with chemotherapy and/or radiotherapy. Examples of biologic agents include immuno-modulating proteins such as cytokines, monoclonal antibodies against tumor antigens, tumor suppressor genes, and cancer vaccines.

Although thousands of potential anti-cancer agents have been evaluated, the treatment of human cancer remains fraught with complications and side effects, which often present an array of suboptimal treatment choices. Despite the great number of anti-neoplastic agents that are used in the clinic for cancer treatment, a need still exists for more effective drugs for treating cancer in a more specific manner. There is thus an urgent and ongoing need to develop new therapeutic approaches to the treatment of cancer, particularly chemical compounds that are easily obtainable and that inhibit the growth/proliferation of cancer tissues while having little or no effect on healthy tissues.

### SUMMARY OF THE INVENTION

The present invention relates to a novel class of anti-cancer compounds that contain a haloacetamide or azide moiety and are, in one embodiment, alkylating agents. These agents, either alone or in a composition, are useful for treating cancer, preventing cancer, delaying the progression of cancer, treating and/or preventing the recurrence of cancer, suppressing, inhibiting or reducing the incidence of cancer, or inducing apoptosis in a cancer cell. Accordingly, the present invention provides a) methods of treating cancer in a subject; b) methods of preventing cancer in a subject; c) methods of delaying the progression of cancer in a subject; d) methods of treating the recurrence of cancer in a subject; e) methods of preventing the recurrence of cancer in a subject; f) methods of suppressing, inhibiting or reducing the incidence of cancer in a subject; and g) methods of inducing apoptosis in a cancer cell, by administering to the subject an anti-cancer compound of the present invention or an analog or metabolite thereof, its N-oxide, ester, pharmaceutically acceptable salt, hydrate, or any combination thereof as described herein.

In one embodiment, the present invention provides an anti-cancer compound represented by the structure of formula I: 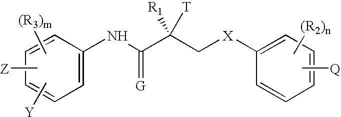
wherein
X is a bond, O, CH₂, NH, S, SO, SO₂, Se, PR, NO or NR;
G is O or S;
T is OH, OR, —NHCOCH₃, NHCOR, or 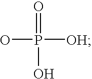
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 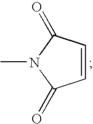
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 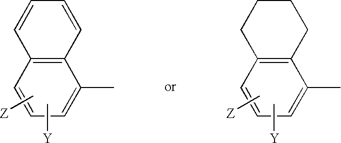
n is an integer of 1-4; and
m is an integer of 1-3.

In one embodiment of the invention, in the anti-cancer compound represented by the structure of formula I, if Z is NO₂, CN, COR, COOH or CONHR, Q is not NHCOCH₂A or N₃.

In one embodiment, the present invention provides an anti-cancer compound represented by the structure of formula I: 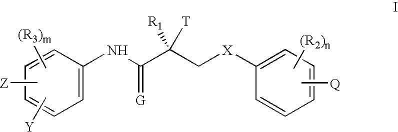
wherein
X is SO or SO₂;
G is O or S;
T is OH, OR, —NHCOCH₃, NHCOR, or 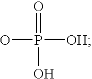
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 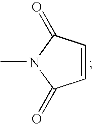
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 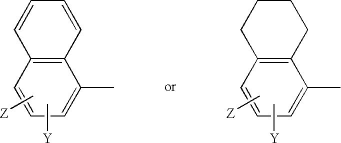
n is an integer of 14; and
m is an integer of 1-3.

In another embodiment, the present invention provides an analog, derivative, isomer, metabolite, pharmaceutically acceptable salt, pharmaceutical product, hydrate or N-oxide of the compound of formula I, or any combination thereof.

In one embodiment, G in compound I is O. In another embodiment, X in compound I is O. In another embodiment, T in compound I is OH. In another embodiment, R₁ in compound I is CH₃. In another embodiment, Z in compound I is NO₂. In another embodiment, Z in compound I is CN. In another embodiment, Y in compound I is CF₃. In another embodiment, Q in compound I is NHCOCH₂Cl. In another embodiment, Q in compound I is NHCOCH₂Br. In another embodiment, Q in compound I is in the para position. In another embodiment, Z in compound I is in the para position. In another embodiment, Y in compound I is in the meta position.

In another embodiment, the present invention provides an anti-cancer compound represented by the structure of formula II: 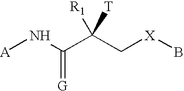
wherein
X is a bond, O, CH₂, NH, S, SO, SO₂, Se, PR, NO or NR;
G is O or S;
T is OH, OR, —NHCOCH₃, NHCOR or 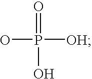
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 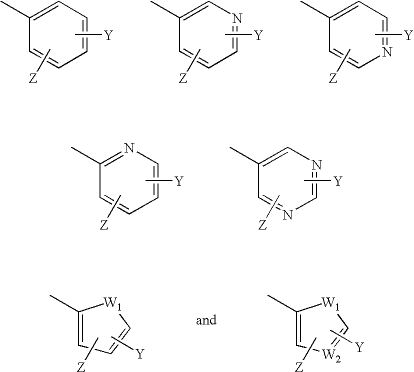
B is a ring selected from: 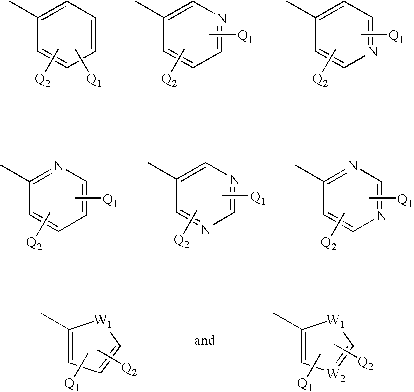
   wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 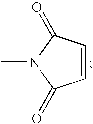
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 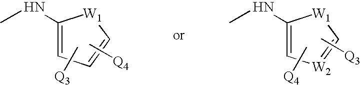
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

In one embodiment of the invention, in the anti-cancer compound represented by the structure of formula II, if Z is NO₂, CN, COR, COOH or CONHR, Q is not NHCOCH₂A or N₃;

In another embodiment, the present invention provides an anti-cancer compound represented by the structure of formula II: 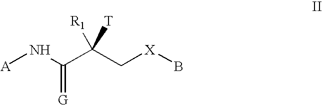
wherein
X is SO or SO₂;
G is O or S;
T is OH, OR, —NHCOCH₃, NHCOR or 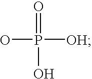
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 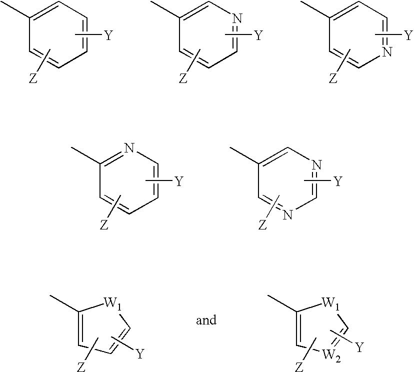
B is a ring selected from: 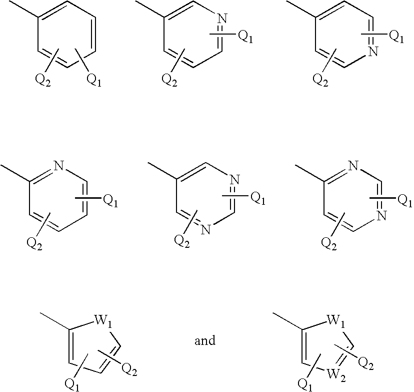
   wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH—CH₂, COCH═CH₂, COCH₂A or 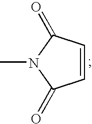
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 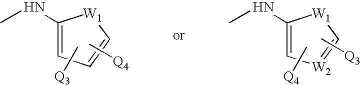
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂is N or NO.

In another embodiment, the present invention provides an analog, derivative, isomer, metabolite, pharmaceutically acceptable salt, pharmaceutical product, hydrate or N-oxide of the compound of formula II, or any combination thereof.

In one embodiment, G in compound II is O. In another embodiment, X in compound II is O. In another embodiment, T in compound II is OH. In another embodiment, R₁ in compound II is CH₃. In another embodiment, Z in compound II is NO₂. In another embodiment, Z in compound II is CN. In another embodiment, Y in compound II is CF₃. In another embodiment, Q₁ in compound II is NHCOCH₂Cl. In another embodiment, Q₁ in compound II is NHCOCH₂Br In another embodiment, Q₁ in compound II is in the para position. In another embodiment, Z in compound II is in the para position. In another embodiment, Y in compound II is in the meta position.

In another embodiment, the present invention provides an anti-cancer compound represented by the structure of formula III: 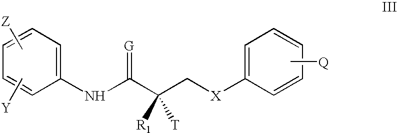
wherein
X is a bond, O, CH₂, NH, S, SO, SO₂, Se, PR, NO or NR;
G is O or S;
T is OH, OR, —NHCOCH₃, NHCOR or 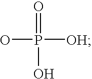
Y is CF₃, F, Cl, Br, I, CN, or SnR₃; one of Z or Q is NO₂, CN, COR, COOH, CONHR, F, Cl, Br or I, and the other is NCS, NHCOCH₂A, N₃, S₂F, N(OH)COR, CONHOH, NHSO₂CH₂A, NHCOCH═CH₂, COCH═CH₂, COCH₂A or 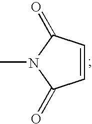
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In one embodiment of the invention, in the anti-cancer compound represented by the structure of formula III, if Z is NO₂, CN, COR, COOH or CONHR, Q is not NHCOCH₂A or N₃;

In another embodiment, the present invention provides an anti-cancer compound represented by the structure of formula III: 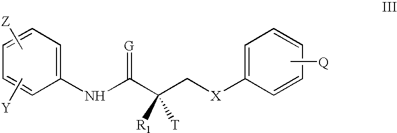
wherein
X is SO or SO₂;
G is O or S;
T is OH, OR, —NHCOCH₃, NHCOR or 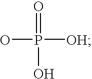
Y is CF₃, F, Cl, Br, I, CN, or SnR₃; one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 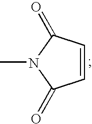
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In another embodiment, the present invention provides an analog, derivative, isomer, metabolite, pharmaceutically acceptable salt, pharmaceutical product, hydrate or N-oxide of the compound of formula III, or any combination thereof.

In one embodiment, G in compound III is O. In another embodiment, X in compound m is O. In another embodiment, T in compound III is OH. In another embodiment, R₁ in compound III is CH₃. In another embodiment, Z in compound m is NO₂. In another embodiment, Z in compound III is CN. In another embodiment, Y in compound III is CF₃. In another embodiment, Q in compound III is NHCOCH₂Cl. In another embodiment, Q in compound III is NHCOCH₂Br. In another embodiment, Q in compound III is in the para position. In another embodiment, Z in compound III is in the para position. In another embodiment, Y in compound III is in the meta position. In 20 another embodiment, G in compound III is O, T is OH, R₁ is CH₃, X is O, Z is NO₂, Y is CF₃, and Q is NCS.

In another embodiment, the present invention provides an anti-cancer compound represented by the structure of formula IV: 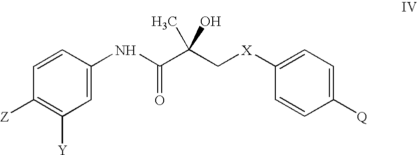
wherein
X is a bond, O, CH₂, NH, S, SO, SO₂, Se, PR, NO or NR;
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 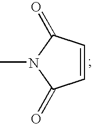
A is F, Cl, Br or I; and
R is alkyl, haloalkyl, dihaloalkyl, trihaloalkyl, CH₂F, CHF₂, CF₃, CF₂CF₃, aryl, phenyl, halogen, alkenyl or OH.

In one embodiment of the invention, in the anti-cancer compound represented by the structure of formula IV, if Z is NO₂, CN, COR, COOH or CONHR , Q is not NHCOCH₂A or N₃.

In another embodiment, the present invention provides an anti-cancer compound represented by the structure of formula IV: 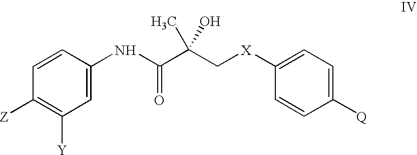
wherein
X is SO or SO₂;
Y is CF₃ F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 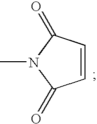
A is F, Cl, Br or I; and
R is alkyl, haloalkyl, dihaloalkyl, trihaloalkyl, CH₂F, CHF₂, CF₃, CF₂CF₃, aryl, phenyl, halogen, alkenyl or OH.

In another embodiment, the present invention provides an analog, derivative, isomer, metabolite, pharmaceutically acceptable salt, pharmaceutical product, hydrate or N-oxide of the compound of formula IV, or any combination thereof.

In one embodiment, X in compound IV is O. In another embodiment, Z in compound IV is NO₂. In another embodiment, Z in compound IV is CN. In another embodiment, Y in compound IV is CF₃. In another embodiment, Q in compound IV is NHCOCH₂Cl. In another embodiment, Q in compound IV is NHCOCH₂Br.

In another embodiment, the present invention provides an anti-cancer compound represented by the structure of formula XIX: 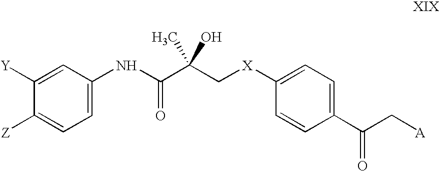
wherein
X is a bond, O, CH₂, NH, S, SO, SO₂, Se, PR, NO or NR;
Y is CF₃, F, Cl, Br, I, CN, or SnR₃;
Z is NO₂, CN, COR, COOH, CONHR, F, Cl, Br or I; and
A is F, Cl, Br or I.

In another embodiment, the present invention provides an analog, derivative, isomer, metabolite, pharmaceutically acceptable salt, pharmaceutical product, hydrate or N-oxide of the compound of formula XIX, or any combination thereof.

In another embodiment, the present invention provides an anti-cancer compound represented by the structure of formula XX: 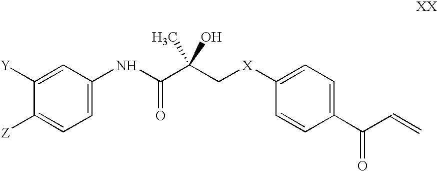
wherein
X is a bond, O, CH₂, NH, S, SO, SO₂, Se, PR, NO or NR;
Y is CF₃, F, Cl, Br, I, CN, or SnR₃; and
Z is NO₂, CN, COR, COOH, CONHR, F, Cl, Br or I.

In another embodiment, the present invention provides an analog, derivative, isomer, metabolite, pharmaceutically acceptable salt, pharmaceutical product, hydrate or N-oxide of the compound of formula XX, or any combination thereof.

In another embodiment, the present invention provides an anti-cancer compound represented by the structure of formula XXI: 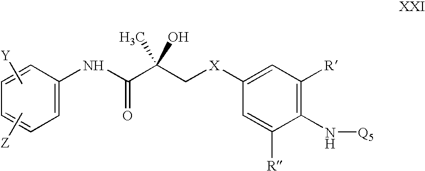
wherein
X is O, S, or SO₂;
Y is CF₃ F, Cl, Br, I, CN, or SnR₃;
Z is NO₂, CN, COR, COOH, CONHR, F, Cl, Br or I;
Q₅ is COCH═CH₂ or COCH₂A;
A is F, Cl, Br or I; and
each of R′ and R″, independently, are H, F, Cl, Br, I or alkyl.

In another embodiment, the present invention provides an analog, derivative, isomer, metabolite, pharmaceutically acceptable salt, pharmaceutical product, hydrate or N-oxide of the compound of formula XXI, or any combination thereof.

In another embodiment, the present invention provides an anti-cancer compound represented by the structure of formula XXII: 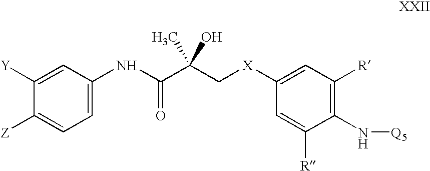
wherein
X is O, S, or SO₂;
Y is CF₃, F, Cl, Br, I, CN, or SnR₃;
Z is NO₂, CN, COR, COOH, CONHR, F, Cl, Br or I;
Q₅ is COCH═CH₂ or COCH₂A;
A is F, Cl, Br or I; and
each of R′ and R″, independently, are H, F, Cl, Br, I or alkyl.

In another embodiment, the present invention provides an analog, derivative, isomer, metabolite, pharmaceutically acceptable salt, pharmaceutical product, hydrate or N-oxide of the compound of formula XXII, or any combination thereof.

In another embodiment, the present invention provides an anti-cancer compound represented by the structure of the formula XXIII: 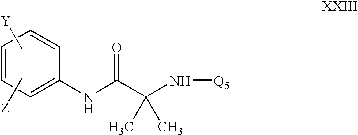
wherein
Y is CF₃, F, Cl, Br, I, CN, or SnR₃;
Z is NO₂, CN, COR, COOH, CONHR, F, Cl, Br or I;
Q₅ is COCH═CH₂ or COCH₂A; and
A is F, Cl, Br or I.

In another embodiment, the present invention provides an analog, derivative, isomer, metabolite, pharmaceutically acceptable salt, pharmaceutical product, hydrate or N-oxide of the compound of formula XXIII, or any combination thereof.

In another embodiment, the present invention provides an anti-cancer compound represented by the structure of the formula XXIV: 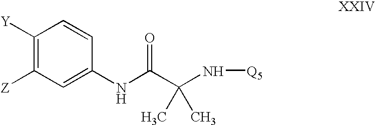
wherein
Y is CF₃, F, Cl, Br, I, CN, or SnR₃;
Z is NO₂, CN, COR, COOH, CONHR, F, Cl, Br or I;
Q₅ is COCH═CH₂ or COCH₂A; and
A is F, Cl, Br or I.

In another embodiment, the present invention provides an analog, derivative, 10 isomer, metabolite, pharmaceutically acceptable salt, pharmaceutical product, hydrate or N-oxide of the compound of formula XXIV, or any combination thereof.

In another embodiment, the present invention provides an anti-cancer compound represented by the structure of the formula XXV: 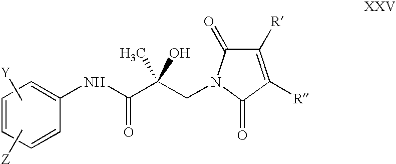
wherein
Y is CF₃, F, Cl, Br, I, CN, or SnR₃;
Z is NO₂, CN, COR, COOH, CONHR, F, Cl, Br or I; and
R′ and R″ are, independently of each other, a hydrogen, alkyl or a halogen.

In another embodiment there is provided according to formula XXV, wherein R′ and R″ together forms a fused ring system represented by the following structure.

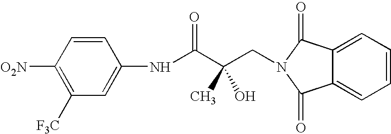

In another embodiment, the present invention provides an analog, derivative, isomer, metabolite, pharmaceutically acceptable salt, pharmaceutical product, hydrate or N-oxide of the compound of formula XXV, or any combination thereof.

In another embodiment, the present invention provides an anti-cancer compound represented by the structure of the formula XXVI: 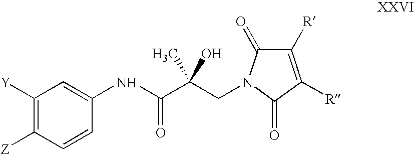
wherein
Y is CF₃, F, Cl, Br, I, CN, or SnR₃;
Z is NO₂, CN, COR, COOH, CONHR, F, Cl, Br or I; and
R′ and R″ are, independently of each other, a hydrogen, alkyl or a halogen.

In another embodiment, the present invention provides an analog, derivative, isomer, metabolite, pharmaceutically acceptable salt, pharmaceutical product, hydrate or N-oxide of the compound of formula XXVI, or any combination thereof.

In one embodiment, the present invention provides a composition comprising the anti-cancer compound of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof.

In another embodiment, the present invention provides a pharmaceutical composition comprising the anti-cancer compound of any of formulae I-IV, XIX-XXVI, XXXIII-XXXIV and any other compund described herein and/or its analog, derivative, isomer, metabolite, pharmaceutical product, hydrate, N-oxide, impurity, prodrug, polymorph, crystal, or any combination thereof and a suitable carrier or diluent.

In another embodiment, the present invention further provides a method of treating a subject suffering from cancer, comprising the step of administering to the subject the anti-cancer compound of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to treat cancer in the subject.

In another embodiment, the present invention provides a method of preventing cancer in a subject, comprising the step of administering to the subject the anti-cancer compound of formulae I-TV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to prevent cancer in the subject.

In another embodiment, the present invention further provides a method of delaying the progression of cancer in a subject suffering from cancer, comprising the step of administering to the subject the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to delay the progression of cancer in the subject.

In another embodiment, the present invention further provides a method of preventing the recurrence of cancer in a subject suffering from cancer, comprising the step of administering to the subject the anti-cancer compound of any of any of formulae I-V, XIX-XXVI, XXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to prevent the recurrence of cancer in the subject.

In another embodiment, the present invention provides a method of treating the recurrence of cancer in a subject suffering from cancer, comprising the step of administering to the subject the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to treat the recurrence of cancer in the subject.

In another embodiment, the present invention provides a method of suppressing, inhibiting or reducing the incidence of cancer in a subject suffering from cancer, comprising the step of administering to the subject the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to suppress, inhibit or reduce the incidence of cancer in the subject.

In another embodiment, the present invention further provides a method of irreversibly binding an anti-cancer compound to a cellular component, comprising the step of contacting a cell comprising the cellular component with the anti-cancer compound of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to irreversibly bind the anti-cancer compound to the cellular component.

In another embodiment, the present invention further provides a method of alkylating a cellular component, comprising the step of contacting a cell comprising the cellular component with the anti-cancer compound of any of any of formulae I-IV, XIX-XXVI, XXXIII-XXXV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to alkylate the cellular component.

In another embodiment, the present invention provides a method of inducing apoptosis in a cancer cell, comprising the step of contacting the cell with the anti-cancer compound of any of any of formulae I-IV, XIX-XXVI, XXXIII-XXV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to induce apoptosis in the cancer cell.

In another embodiment, the present invention provides process for preparing an anti-cancer compound represented by the structure of formula I: 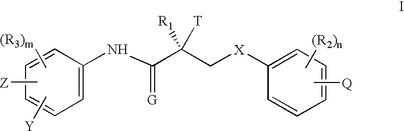
wherein
X is a bond, O, CH₂, NH, S, SO, SO₂, Se, PR, NO or NR;
G is O or S;
T is OH, OR, —NHCOCH₃, NHCOR, or 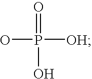
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 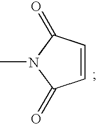
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 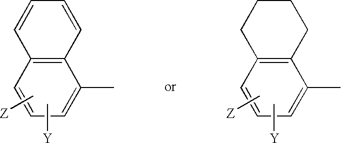
n is an integer of 1-4; and
m is an integer of 1-3;
   the process comprising the step of coupling a compound of formula VIII: 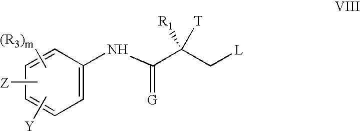
   wherein Z, Y, G, R₁, T, R₃ and m are as defined above and L is a leaving group, with a compound of formula IX: 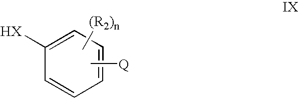
   wherein Q, X R₂ and n are as defined above.

In one embodiment of the invention, in the anti-cancer compound represented by the structure of formula I, if Z is NO₂, CN, COR, COOH or CONHR, Q is not NHCOCH₂A or N₃;

In another embodiment, the present invention provides process for preparing an anti-cancer compound represented by the structure of formula I: 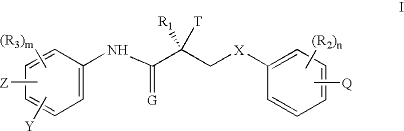
wherein
X is SO or SO₂;
G is O or S;
T is OH, OR, —NHCOCH₃, NHCOR, or 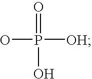
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 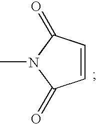
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF2, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 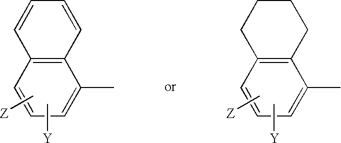
n is an integer of 1-4; and
m is an integer of 1-3;
   the process comprising the step of coupling a compound of formula VIII: 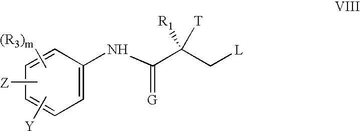
   wherein Z, Y, G, R₁, T, R₃ and m are as defined above and L is a leaving group, with a compound of formula IX: 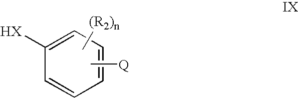
   wherein Q, X R₂ and n are as defined above.

In one embodiment of the invention, in the anti-cancer compound represented by the structure of formula I, if Z is NO₂, CN, COR, COOH or CONHR, Q is not NHCOCH₂A or N₃;

In one embodiment, the process further comprises the step of converting the Is anti-cancer compound to its analog, isomer, metabolite, derivative, pharmaceutically acceptable salt, pharmaceutical product, N-oxide, hydrate or any combination thereof

In another embodiment, the present invention provides process for preparing an anti-cancer compound represented by the structure of formula II: 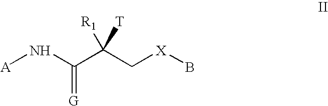
wherein
X is a bond, O, CH₂, NH, S, SO, SO₂, Se, PR, NO or NR;
G is O or S;
T is OH, OR, —NHCOCH₃, NHCOR or 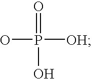
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 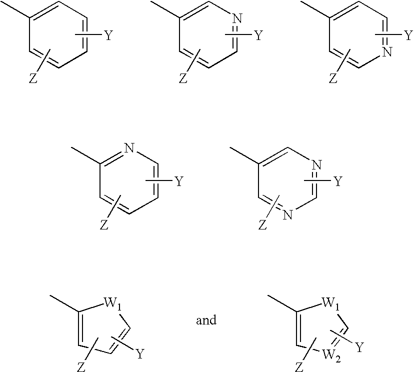
B is a ring selected from: 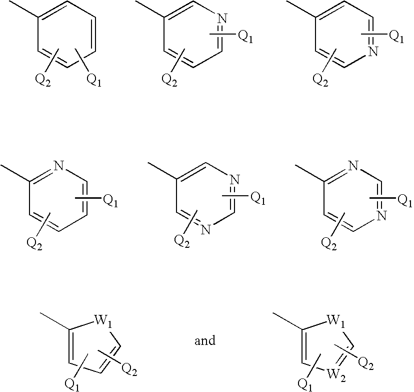
   wherein A and B cannot simultaneously be a benzene ring;
Y is CF₃, F, I, Br, Cl, CN CR₃ or SnR₃;
one of Z or Q, is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 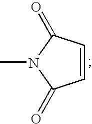
A is F, Cl, Br or I
Q2 is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 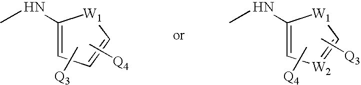
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;
   the process comprising the step of coupling a compound of formula XIII: 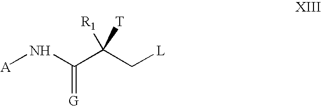
   wherein A, G, R₁ and T are as defined above and L is a leaving group, with a compound of formula HX-B wherein B and X are as defined above.

In one embodiment of the invention, in the anti-cancer compound represented by the structure of formula II, if Z is NO₂, CN, COR, COOH or CONHR, Q is not NHCOCH₂A or N₃;

In one embodiment, the process further comprises the step of converting the anti-cancer compound to its analog, isomer, metabolite, derivative, pharmaceutically acceptable salt, pharmaceutical product, N-oxide, hydrate or any combination thereof.

In another embodiment, the present invention provides process for preparing an anti-cancer compound represented by the structure of formula II: 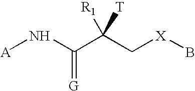
wherein
X is SO or SO₂;
G is O or S;
T is OH, OR, —NHCOCH₃, NHCOR or 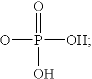
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 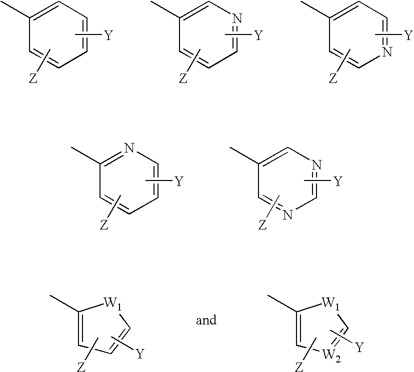
B is a ring selected from: 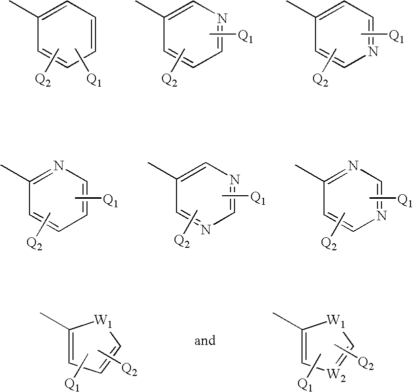
   wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 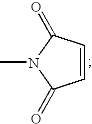
A is F, Cl, Br or I
Q² is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 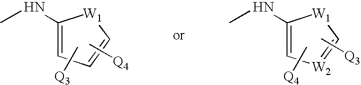
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;
   the process comprising the step of coupling a compound of formula XIII: 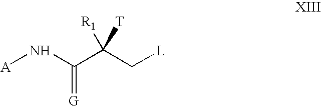
   wherein A, G, R₁ and T are as defined above and L is a leaving group, with a compound of formula HX-B wherein B and X are as defined above.

In another embodiment, the present invention provides process for preparing an anti-cancer compound represented by the structure of formula III: 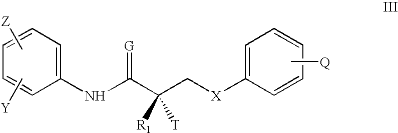
wherein
X is a bond, O, CH₂, NH, S, SO, SO₂, Se, PR, NO or NR;
G is O or S;
T is OH, OR, —NHCOCH₃, NHCOR, or 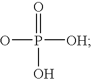
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 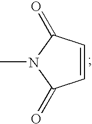
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃;
   the process comprising the step of coupling a compound of formula XIV: 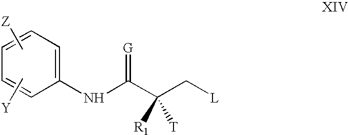
   wherein Z, Y, G R₁ and T are as defined above and L is a leaving group, with a compound of formula XV: 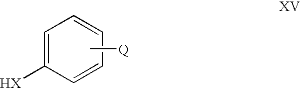
   wherein Q and X are as defined above.

In one embodiment of the invention, in the anti-cancer compound represented by the structure of formula III, if Z is NO₂, CN, COR, COOH or CONHR, Q is not NHCOCH₂A or N₃;

In another embodiment, the present invention provides process for preparing an S anti-cancer compound represented by the structure of formula III: 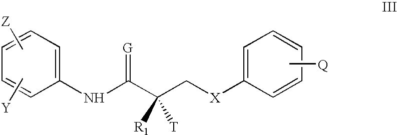
wherein
X is SO or SO₂;
G is O or S;
T is OH, OR, —NHCOCH₃, NHCOR, or 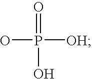
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 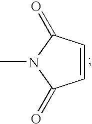
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃;
   the process comprising the step of coupling a compound of formula XIV: 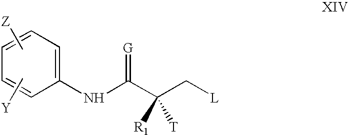
   wherein Z, Y, G R₁ and T are as defined above and L is a leaving group, with a compound of formula XV: 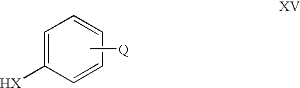
   wherein Q and X are as defined above.

In one embodiment, the process further comprises the step of converting the anti-cancer compound to its analog, isomer, metabolite, derivative, pharmaceutically acceptable salt, pharmaceutical product, N-oxide, hydrate or any combination thereof.

In another embodiment, the present invention provides process for preparing an anti-cancer compound represented by the structure of formula IV: 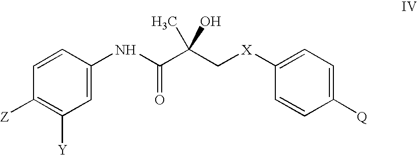
wherein
X is a bond, O, CH₂, NH, S, SO, SO₂, Se, PR, NO or NR;
Y is CF₃ F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 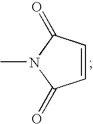
A is F, Cl, Br or I; and
R is alkyl, haloalkyl, dihaloalkyl, trihaloalkyl, CH₂F, CHF₂, CF₃, CF₂CF₃, aryl, phenyl, halogen, alkenyl or OH;
   the process comprising the step of coupling an amide of formula XVII: 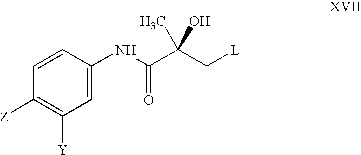
   wherein Z and Y are as defined above and L is a leaving group, with a compound of formula XVIII: 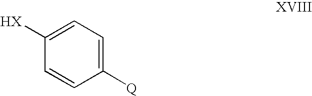
   wherein Q and X R₂ are as defined above.

In one embodiment of the invention, in the anti-cancer compound represented by the structure of formula IV, if Z is NO₂, CN, COR, COOH or CONHR, Q is not NHCOCH₂A or N₃;

In another embodiment, the present invention provides process for preparing an anti-cancer compound represented by the structure of formula IV: 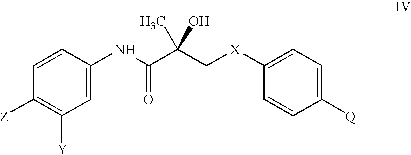
wherein
X is SO or SO₂;
Y is CF₃ F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 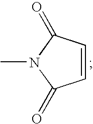
A is F, Cl, Br or I; and
R is alkyl, haloalkyl, dihaloalkyl, trihaloalkyl, CH₂F, CHF₂, CF₃, CF₂CF₃, aryl, phenyl, halogen, alkenyl or OH;
   the process comprising the step of coupling an amide of formula XVII: 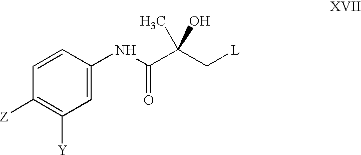
   wherein Z and Y are as defined above and L is a leaving group, with a compound of formula XVIII: 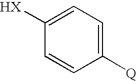
   wherein Q and X R₂ are as defined above.

In another embodiment, the present invention provides process for preparing an anti-cancer compound represented by the structure of the formula XXI: 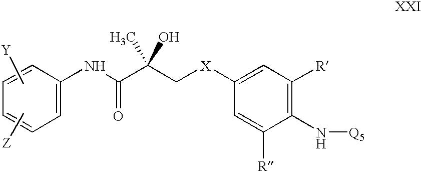
wherein
X is O, S, or SO;
Y is CF₃, F, Cl, Br, I, CN, or SnR₃;
Z is NO₂, CN, COR, COOH, CONHR, F, Cl, Br or I;
Q₅ is COCH═CH₂ or COCH₂A;
A is F, Cl, Br or I; and
each of R′ and R″, independently, are H, F, Cl, Br, I or alkyl.
   the process comprising the steps of:
coupling a compound represented by the structure of the formula XXVII: 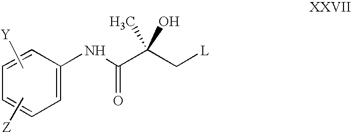
   wherein Y and Z are defined above and L is a leaving group, with a compound represented by the structure of the formula XXVIII: 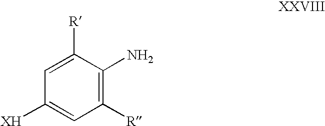
   wherein R′, R″ and X are defined above,
to prepare a compound represented by the structure of the formula XXIX: 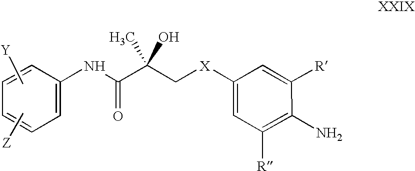
   wherein R′, R″, X, Y and Z are defined above; and
reacting the compound formula XXIX with A-Q₅, wherein A and Q₅ are defined above, to obtain a compound represented by the structure of the formula XXI.

In another embodiment, the present invention provides process for preparing an anti-cancer compound represented by the structure of the formula XXIII: 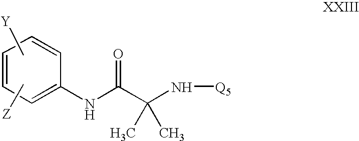
wherein
Y is CF₃, F, Cl, Br, I, CN, or SnR₃;
Z is NO₂, CN, COR, COOH, CONHR, F, Cl, Br or I;
Q₅ is COCH═CH₂ or COCH₂A; and
A is F, Cl, Br or I,
the process comprising the step of:
reacting a compound represented by the structure of the formula XXX: 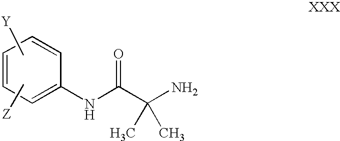
   wherein Y and Z are defined above,
with A-Q₅, wherein A and Q₅ are defined above, to obtain a compound represented by the structure of the formula XXIII.

In another embodiment, the present invention provides process for preparing an anti-cancer compound represented by the structure of the formula XXV: 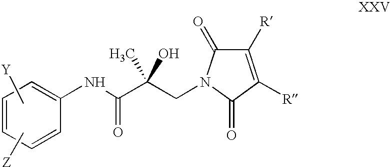
wherein
Y is CF₃, F, Cl, Br, I, CN, or SnR₃;
Z is NO₂, CN, COR, COOH, CONHR, F, Cl, Br or I; and
R′ and R″ are, independently of each other, a hydrogen, alkyl or a halogen, the process comprising the step of:
reacting a compound represented by the structure of the formula XXXI: 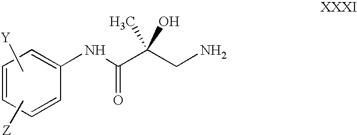
   wherein Y and Z are defined above, with a an unsubstituted or a substituted phthalic anhydride represented by the structure of the formula XXXII: 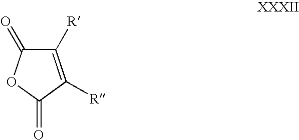
   wherein R′ and R″ are defined above, to obtain a compound represented by the structure of the formula XXV.

In one embodiment, the process further comprises the step of purifying the anti-cancer compound using a mixture of ethanol and water. In another embodiment, the process further comprises the step of converting the anti-cancer compound to its analog, isomer, metabolite, derivative, pharmaceutically acceptable salt, pharmaceutical product, N-oxide, hydrate or any combination thereof.

The novel anti-cancer compounds of the present invention, either alone or as a pharmaceutical composition, are useful for a) treating cancer in a subject; b) preventing cancer in a subject; c) delaying the progression of cancer in a subject; d) treating the recurrence of cancer in a subject; e) preventing the recurrence of cancer in a subject; f) suppressing, inhibiting or reducing the incidence of cancer in a subject; and/or g) inducing apoptosis in a cancer cell.

### BRIEF DESCRIPTION OF THE DRAWINGS

The present invention will be understood and appreciated more fully from the following detailed description taken in conjunction with the appended drawings in which:

FIG. 1A: Cytotoxicity of Compound A (bromoacetemido substituted) in different cell lines.

FIG 1B: Cytotoxicity of Compound B (chlorocetemido substituted) in different cell lines.

FIG 1C: Cytotoxicity of compound S-NTBA in different cell lines.

FIG. 2A: Growth Curve: Effect of Compound A (bromoacetamido substituted) on growth of different cell lines.

FIG. 2B: Growth Curve: Effect of Compound B (chloroacetamido substituted) on growth of different cell lines.

FIGS. 3A, B: Tunnel Assay: Top panel: LNCAP cells exposed to Compound A for 24 hours. Bottom Panel: 0.1% vehicle control.

FIGS. 4A-D: show inhibition of cell growth by compounds XXXIII-XXXVI of the invention.

### DETAILED DESCRIPTION OF THE INVENTION

The present invention relates to a novel class of anti-cancer compounds, which contain a haloacetamide or azide moiety and are, in one embodiment, alkylating agents. The compounds of the present invention, either alone or in a composition, are useful for treating cancer, preventing cancer, delaying the progression of cancer, treating and/or preventing the recurrence of cancer, suppressing, inhibiting or reducing the incidence of cancer, or inducing apoptosis in a cancer cell. Accordingly, the present invention provides a) methods of treating cancer in a subject; b) methods of preventing cancer in a subject; c) methods of delaying the progression of cancer in a subject; d) methods of treating the recurrence of cancer in a subject; e) methods of preventing the recurrence of cancer in a subject; f) methods of suppressing, inhibiting or reducing the incidence of cancer in a subject; and g) methods of inducing apoptosis in a cancer cell; by administering to the subject an anti-cancer compound of the present invention or an analog or metabolite thereof, its N-oxide, ester, pharmaceutically acceptable salt, hydrate, or any combination thereof as described herein.

In one embodiment, the present invention provides an anti-cancer compound represented by the structure of formula I: 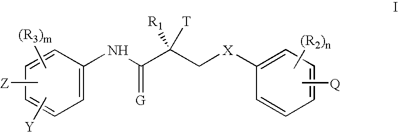
wherein
X is a bond, O, CH₂, NH, S, SO, SO₂, Se, PR, NO or NR;
G is O or S;
T is OH, OR, —NHCOCH₃, NHCOR, or 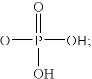
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 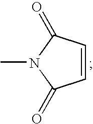
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fised ring system represented by the structure: 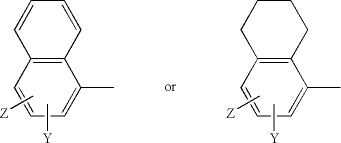
n is an integer of 1-4; and
m is an integer of 1-3.

In one embodiment of the invention, in the anti-cancer compound represented by the structure of formula I, if Z is NO₂, CN, COR, COOH or CONHR, Q is not NHCOCH₂A or N₃;

In one embodiment, the present invention provides an anti-cancer compound represented by the structure of formula I: 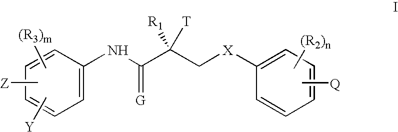
wherein
X is SO or SO₂;
G is O or S;
T is OH, OR, —NHCOCH₃, NHCOR, or 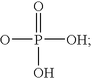
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 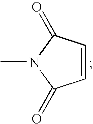
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, s COOH, CONH, CF₃ or SnR₃, or R₃ together with the benzene ring to which it is attached forms a fused ring system represented by the structure: 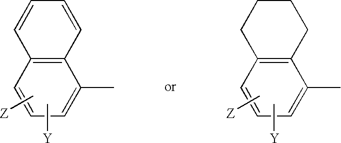
n is an integer of 1-4; and
m is an integer of 1-3.

In one embodiment of the invention, in the anti-cancer compound represented by the structure of formula I, if Z is NO₂, CN, COR, COOH or CONHR, Q is not NHCOCH₂A or N₃;

In one embodiment, this invention provides an analog of the compound of formula I. In another embodiment, this invention provides a derivative of the compound of formula I. In another embodiment, this invention provides an isomer of the compound of formula I. In another embodiment, this invention provides a metabolite of the compound of formula I. In another embodiment, this invention provides a pharmaceutically acceptable salt of the compound of formula I. In another embodiment, this invention provides a pharmaceutical product of the compound of formula I. In another embodiment, this invention provides a hydrate of the compound of formula I. In another embodiment, this invention provides an N-oxide of the compound of formula I. In another embodiment, this invention provides a combination of any of an analog, derivative, metabolite, isomer, pharmaceutically acceptable salt, pharmaceutical product, hydrate or N-oxide of the compound of formula I.

In one embodiment, G in compound I is O. In another embodiment, X in compound I is O. In another embodiment, T in compound I is OH. In another embodiment, R₁ in compound I is CH₃. In another embodiment, Z in compound I is NO₂. In another embodiment, Z in compound I is CN. In another embodiment, Y in compound I is CF₃. In another embodiment, Q in compound I is NHCOCH₂Cl. In another embodiment, Q in compound I is NHCOCH₂Br. In another embodiment, Q in compound I is N₃. In another embodiment, Q in compound I is COCH═CH₂. In another embodiment, Q in compound I is COCH₂A. In another embodiment, Q in compound I is

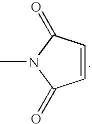

In another embodiment, Q in compound I is in the para position. In another embodiment, Z in compound I is in the para position. In another embodiment, Y in compound I is in the meta position.

In another embodiment, the present invention provides an anti-cancer compound represented by the structure of formula II: 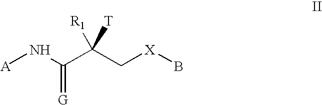
wherein
X is a bond, O, CH₂, NH, S, SO, SO₂, Se, PR, NO or NR;
G is O or S;
T is OH, OR, —NHCOCH₃, NHCOR or 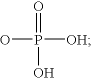
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 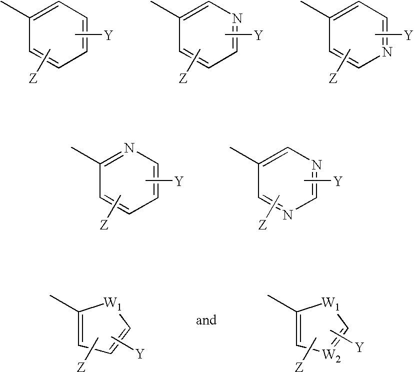
B is a ring selected from: 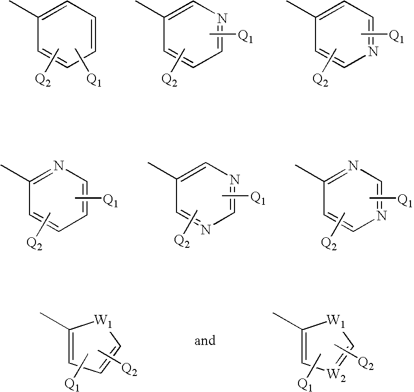
   wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 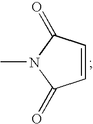
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 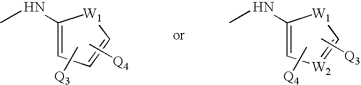
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

In one embodiment of the invention, in the anti-cancer compound represented by the structure of formula I, if Z is NO₂, CN, COR, COOH or CONHR, Q is not NHCOCH₂A or N₃;

In another embodiment, the present invention provides an anti-cancer compound represented by the structure of formula II: 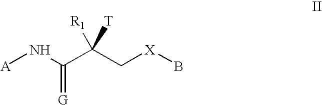
wherein
X is SO or SO₂;
G is O or S;
T is OH, OR, —NHCOCH₃, NHCOR or 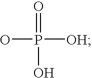
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 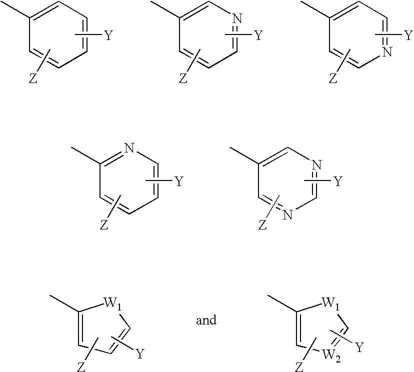
B is a ring selected from: 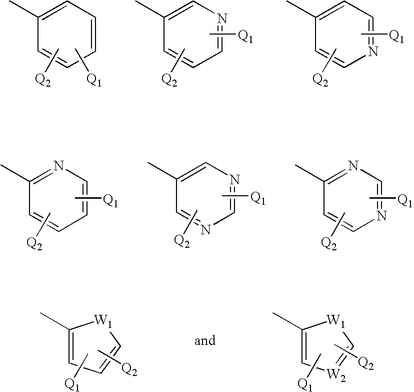
   wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH2A or 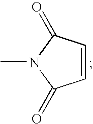
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONH, CONHR, NTHCSCH₃, NHCSCF₃, NHCSR NHSO₂CH₃, NHSO₂R, OR, COR, OCOR, OSO₂R, SO₂R, SR, 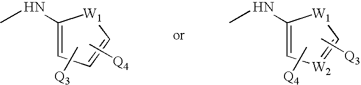
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONH, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

In one embodiment, this invention provides an analog of the compound of formula II. In another embodiment, this invention provides a derivative of the compound of formula II. In another embodiment, this invention provides an isomer of the compound of formula II. In another embodiment, this invention provides a metabolite of the compound of formula II. In another embodiment, this invention provides a pharmaceutically acceptable salt of the compound of formula II. In another embodiment, this invention provides a pharmaceutical product of the compound of formula II. In another embodiment, this invention provides a hydrate of the compound of formula II. In another embodiment, this invention provides an N-oxide of the compound of formula II. In another embodiment, this invention provides a combination of any of an analog, derivative, metabolite, isomer, pharmaceutically acceptable salt, pharmaceutical product, hydrate or N-oxide of the compound of formula II.

In one embodiment, G in compound II is O. In another embodiment, X in compound II is O. In another embodiment, T in compound II is OH. In another embodiment, R₁ in compound II is CH₃. In another embodiment, Z in compound II is NO₂. In another embodiment, Z in compound II is CN. In another embodiment, Y in compound II is CF₃. In another embodiment, Q₁ in compound II is NHCOCH₂Cl. In another embodiment, Q₁ in compound II is NHCOCH₂Br. In another embodiment, Q₁ in compound II is N₃. In another embodiment, Q₁ in compound II is COCH═CH₂. In another embodiment, Q₁ in compound II is COCH₂A. In another embodiment, Q₁ in compound II is 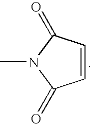
In another embodiment, Q₁ in compound II is in the para position. In another embodiment, Z in compound II is in the para position. In another embodiment, Y in compound II is in the meta position.

In another embodiment, the present invention provides an anti-cancer compound represented by the structure of formula III: 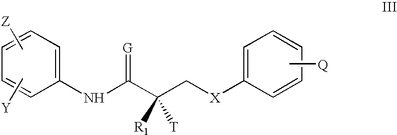
wherein
X is a bond, O, CH₂, NH, S, SO, SO₂, Se, PR, NO or NR;
G is O or S;
T is OH, OR, —NHCOCH₃, NHCOR or 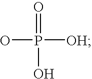
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH2, COCH═CH₂, COCH₂A or 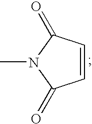
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In one embodiment of the invention, in the anti-cancer compound represented by the structure of formula I, if Z is NO₂, CN, COR, COOH or CONHR, Q is not NHCOCH₂A or N₃;

In another embodiment, the present invention provides an anti-cancer compound represented by the structure of formula III: 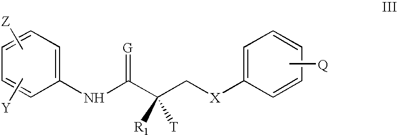
wherein
X is SO or SO₂;
G is O or S;
T is OH, OR, —NHCOCH₃, NHCOR or 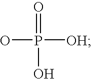
Y is CF3, F, Cl, Br, I, CN, or SnR₃;
one of Z or Q is NO₂, CN, COR, COOH, CONIR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 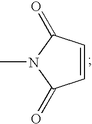
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and

R₁ is CH₃, CH₂F, CHF₂, CF₃, CH₂CH₃, or CF₂CF₃.

In one embodiment of the invention, in the anti-cancer compound represented by the structure of formula III, if Z is NO₂, CN, COR, COOH or CONHR, Q is not NHCOCH₂A or N₃;

In one embodiment, G in compound III is O. In another embodiment, X in compound III is O. In another embodiment, T in compound III is OH. In another embodiment, R₁ in compound III is CH₃. In another embodiment, Z in compound III is NO₂. In another embodiment, Z in compound III is CN. In another embodiment, Y in compound III is CF₃. In another embodiment, Q in compound m is NHCOCH₂Cl. In another embodiment, Q in compound III is NHCOCH₂Br. In another embodiment, Q in compound III is N₃. In another embodiment, Q in compound III is COCH═CH₂. In another embodiment, Q in compound III is COCH₂A. In another embodiment, Q in compound III is 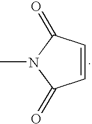
In another embodiment, Q in compound III is in the para position. In another embodiment, Z in compound III is in the para position. In an other embodiment, Y in compound III is in the meta position. In another embodiment, G in compound Ill is O, T is OH, R₁ is CH₃, X is O, Z is NO₂, Y is CF₃, and Q is NCS.

In one embodiment, G in compound III is O. In another embodiment, X in compound III is O. In another embodiment, T in compound III is OH. In another embodiment, R₁ in compound III is CH₃. In another embodiment, Z in compound III is NO₂. In another embodiment, Z in compound III is CN. In another embodiment, Y in compound III is CF₃. In another embodiment, Q in compound III is NCS. In another embodiment, Q in compound III is in the para position. In another embodiment, Z in compound III is in the para position. In another embodiment, Y in compound III is in the meta position. In another embodiment, G in compound III is O, T is OH, R₁ is CH₃, X is O, Z is NO₂, Y is CF₃, and Q is NCS.

In another embodiment, the present invention provides an anti-cancer compound represented by the structure of formula IV: 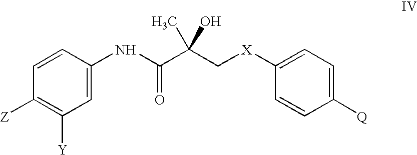
wherein
X is a bond, O, CH₂, NH₄, S, SO, SO₂, Se, PR, NO or NR;
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 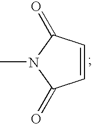
A is F, Cl, Br or I; and
R is alkyl, haloalkyl, dihaloalkyl, trihaloalkyl, CH₂F, CHF₂, CF₃, CF₂CF₃, aryl, phenyl, halogen, alkenyl or OH.

In one embodiment of the invention, in the anti-cancer compound represented by the structure of formula IV, if Z is NO₂, CN, COR, COOH or CONHR, Q is not NHCOCH₂A or N₃;

In another embodiment, the present invention provides an anti-cancer compound represented by the structure of formula IV: 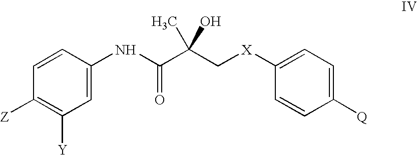
wherein
X is SO or SO₂;
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 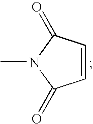
A is F, Cl, Br or I; and
R is alkyl, haloalkyl, dihaloalkyl, trihaloalkyl, CH₂F, CHF₂, CF₃, CF₂CF₃, aryl, phenyl, halogen, alkenyl or OH.

In one embodiment, this invention provides an analog of the compound of formula IV. In another embodiment, this invention provides a derivative of the compound of formula IV. In another embodiment, this invention provides an isomer of the compound of formula IV. In another embodiment, this invention provides a metabolite of the compound of formula IV. In another embodiment, this invention provides a pharmaceutically acceptable salt of the compound of formula IV. In another embodiment, this invention provides a pharmaceutical product of the compound of formula IV. In another embodiment, this invention provides a hydrate of the compound of formula IV. In another embodiment, this invention provides an N-oxide of the compound of formula IV. In another embodiment, this invention provides a combination of any of an analog, derivative, metabolite, isomer, pharmaceutically acceptable salt, pharmaceutical product, hydrate or N-oxide of the compound of formula IV.

In one embodiment, X in compound IV is O. In another embodiment, Z in compound IV is NO₂. In another embodiment, Z in compound IV is CN. In another embodiment, Y in compound IV is CF₃. In another embodiment, Q in compound IV is NHCOCH₂Cl. In another embodiment, Q in compound IV is NHCOCH₂Br. In another embodiment, Q in compound IV is N₃. In another embodiment, Q in compound IV is COCH═CH₂. In another embodiment, Q in compound IV is COCH₂A. In another embodiment, Q in compound IV is

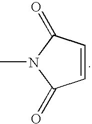

In another embodiment, the present invention provides an anti-cancer compound represented by the structure of formula XIX: 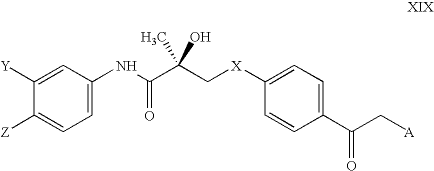
wherein
X is a bond, O, CH₂, NH, S, SO, SO₂, Se, PR, NO or NR;
Y is CF₃, F, Cl, Br, I, CN, or SnR₃;
Z is NO₂, CN, COR, COOH, CONHR, F, Cl, Br or I; and
A is F, Cl, Br or I.

In one embodiment of the invention, the compound of formula XIX is a ketone alkylating agent. In another embodiment, the alkylating group is an alpha halo amide.

In another embodiment, the present invention provides an analog, derivative, isomer, metabolite, pharmaceutically acceptable salt, pharmaceutical product, hydrate or N-oxide of the compound of formula XIX, or any combination thereof.

In another embodiment, the present invention provides an anti-cancer compound represented by the structure of formula XX: 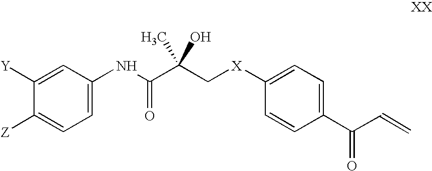
wherein
X is a bond, O, CH₂, NH, S, SO, SO₂, Se, PR, NO or NR;
Y is CF₃, F, Cl, Br, I, CN, or SnR₃; and
Z is NO₂, CN, COR, COOH, CONHR, F, Cl, Br or I.

In one embodiment of the invention, the compound of formula XIX is a ketone alkylating agent. In another embodiment, the alkylating group is an alpha beta unsaturated amide.

In another embodiment, the present invention provides an analog, derivative, isomer, metabolite, pharmaceutically acceptable salt, pharmaceutical product, hydrate or N-oxide of the compound of formula XX, or any combination thereof.

In another embodiment, the present invention provides an anti-cancer compound represented by the structure of formula XXI: 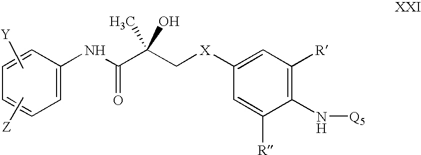
wherein
X is O, S, or SO₂;
Y is CF₃, F, Cl, Br, I, CN, or SnR₃;
Z is NO₂, CN, COR, COOH, CONHR, F, Cl, Br or I;
Q₅ is COCH═CH₂ or COCH₂A;
A is F, Cl, Br or I; and
each of R′ and R″, independently, are H, F, Cl, Br, I or alkyl.

In one embodiment of the invention, the B-ring (right ring) of the compound of formula XXI is mono or di substituted. In another embodiment, the mono or di substituted B-ring prevents metabolism of the alkylating functional group, either alpha halo amide or alpha beta unsaturated amide. In another embodiment, at least one of R′ and R″ is not hydrogen. In another embodiment, the alkyl is CH₃.

In another embodiment, the present invention provides an analog, derivative, isomer, metabolite, pharmaceutically acceptable salt, pharmaceutical product, hydrate or N-oxide of the compound of formula XXI, or any combination thereof.

In another embodiment, the present invention provides an anti-cancer compound represented by the structure of formula XXII: 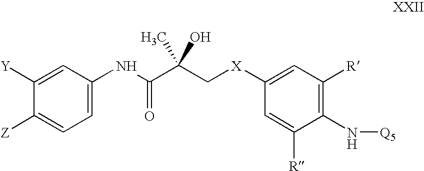
wherein
X is O, S, or SO₂;
Y is CF₃, F, Cl, Br, I, CN, or SnR₃;
Z is NO₂, CN, COR, COOH, CONHR, F, Cl, Br or I;
Q₅ is COCH═CH₂ or COCH₂A;
A is F, Cl, Br or I; and
each of R′ and R″, independently, are H, F, Cl, Br, I or alkyl.

In one embodiment of the invention, the B-ring (right ring) of the compound of formula XXII is mono or di substituted. In another embodiment, the mono or di substituted B-ring prevents metabolism of the alkylating functional group, either alpha halo amide or alpha beta unsaturated amide. In another embodiment, at least one of R′ and R″ is not hydrogen. In another embodiment, the alkyl is CH₃.

In another embodiment, the present invention provides an analog, derivative, isomer, metabolite, pharmaceutically acceptable salt, pharmaceutical product, hydrate or N-oxide of the compound of formula XXII, or any combination thereof.

In another embodiment, the present invention provides an anti-cancer compound represented by the structure of the formula XXIII: 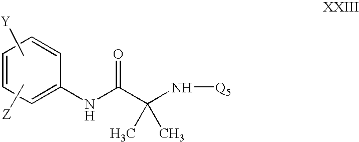
wherein
Y is CF₃, F, Cl, Br, I, CN, or SnR₃;
Z is NO₂, CN, COR, COOH, CONHR, F, Cl, Br or I;
Q₅ is COCH═CH₂ or COCH₂A; and
A is F, Cl, Br or I.

In one embodiment of the invention, the compound of formula XXIII is a flutamide-based alkylating agent. In another embodiment, the alkylating group may be, inter alia, an alpha halo amide or an alpha beta-unsaturated amide.

In one embodiment of the invention, Q₅ is COCH═CH₂. In another embodiment, Q₅ is COCH₂A.

In another embodiment, the present invention provides an analog, derivative, isomer, metabolite, pharmaceutically acceptable salt, pharmaceutical product, hydrate or N-oxide of the compound of formula XXIII, or any combination thereof.

In another embodiment, the present invention provides an anti-cancer compound represented by the structure of the formula XXIV: 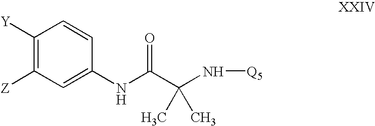
wherein
Y is CF₃, F, Cl, Br, I, CN, or SnR₃;
Z is NO₂, CN, COR, COOH, CONHR, F, Cl, Br or I;
Q₅ is COCH═CH₂ or COCH₂A; and
A is F, Cl, Br or I.

In one embodiment of the invention, the compound of formula XXIV is a flutanide-based alkylating agent. In another embodiment, the alkylating group may be, inter alia, an alpha halo amide or an alpha beta-unsaturated amide.

In one embodiment of the invention, Q₅ is COCH═CH₂. In another embodiment, Q₅ is COCH₂A.

In another embodiment, the present invention provides an analog, derivative, isomer, metabolite, pharmaceutically acceptable salt, pharmaceutical product, hydrate or N-oxide of the compound of formula XXIV, or any combination thereof.

In another embodiment, the present invention provides an anti-cancer compound represented by the structure of the formula XXV: 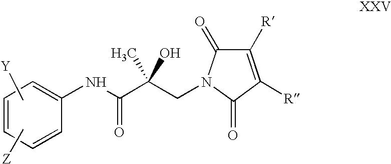
wherein
Y is CF₃, F, Cl, Br, I, CN, or SnR₃;
Z is NO₂, CN, COR, COOH, CONS, F, Cl, Br or I; and
R′ and R″ are, independently of each other, a hydrogen, alkyl or a halogen.

In one embodiment of the invention, the compound of formula XXV is an alkylating agent. In another embodiment, the unsaturated B-ring (imide ring) may be substituted or unsubstituted. In another embodiment, the substituted or unsubstituted unsaturated B-ring (imide ring) acts as an alkylating group.

In another embodiment, the present invention provides an analog, derivative, isomer, metabolite, pharmaceutically acceptable salt, pharmaceutical product, hydrate or N-oxide of the compound of formula XXV, or any combination thereof.

In another embodiment, the present invention provides an anti-cancer compound represented by the structure of the formula XXVI: 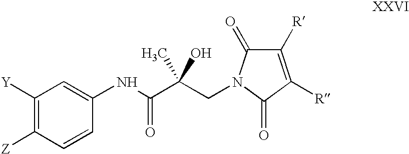
wherein
Y is CF₃, F, Cl, Br, I, CN, or SnR₃;
Z is NO₂, CN, COR, COOH, CONHR, F, Cl, Br or I; and
R′ and R″ are, independently of each other, a hydrogen, alkyl or a halogen.

In one embodiment of the invention, the compound of formula XXVI is an alkylating agent. In another embodiment, the unsaturated B-ring (imide ring) may be substituted or unsubstituted. In another embodiment, the substituted or unsubstituted unsaturated B-ring (imide ring) acts as an alkylating group.

In another embodiment, the present invention provides an analog, derivative, isomer, metabolite, pharmaceutically acceptable salt, pharmaceutical product, hydrate or N-oxide of the compound of formula XXVI, or any combination thereof.

In one embodiment, the invention provides an anti-cancer compound represented by the structure of the formula XXXIII:

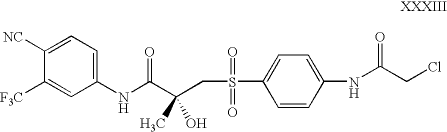

The substituent R is defined herein as an alkyl, haloalkyl, dihaloalkyl, trihaloalkyl, CH₂F, CHF₂, CF₃, CF₂CF₃; aryl, phenyl, halogen, alkenyl, or hydroxyl (OH).

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “haloalkyl” group refers to an alkyl group as defined above, which is substituted by one or more halogen atoms, e.g. by F, Cl, Br or I.

An “aryl” group refers to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers to an OH group. An “alkenyl” group refers to a group having at least one carbon to carbon double bond. A halo group refers to F, Cl, Br or I.

An “arylalkyl” group refers to an alkyl bound to an aryl, wherein alkyl and aryl are as defined above. An example of an aralkyl group is a benzyl group.

According to embodiments of the invention, the phrase “protecting group” as used herein means temporary modifications of a potentially reactive functional group which protect it from undesired chemical transformations.

In one embodiment of the invention, the protecting group may be, inter alia, an amino protecting group. In one embodiment of the invention, an amino protecting group may be, inter alia, benzyloxycarbonyl (Cbz), 9-fluorenylmethyloxycarbonyl (Fmoc), t-butyloxycarbonyl, (tBoc), biphenylisopropyloxycarbonyl, t-amyloxycarbonyl, isobomyloxycarbonyl, alpha-dimethyl-3,5-dimethoxybenzyloxycarbonyl, 2-cyano-t-butyloxycarbonyl, acetate (Ac), —COOAlkyl or any other appropriate amino protecting group.

As contemplated herein, the present invention relates to the use of an anti-cancer compound and/or its analog, derivative, isomer, metabolite, pharmaceutically acceptable salt, pharmaceutical product, hydrate, N-oxide, or combinations thereof. In one embodiment, the invention relates to the use of an analog of the anti-cancer compound. In another embodiment, the invention relates to the use of a derivative of the anti-cancer compound. In another embodiment, the invention relates to the use of an isomer of the anti-cancer compound. In another embodiment, the invention relates to the use of a metabolite of the anti-cancer compound. In another embodiment, the invention relates to the use of a pharmaceutically acceptable salt of the anti-cancer compound. In another embodiment, the invention relates to the use of a pharmaceutical product of the anti-cancer compound. In another embodiment, the invention relates to the use of a hydrate of the anti-cancer compound. In another embodiment, the invention relates to the use of an N-oxide of the anti-cancer compound. In another embodiment, the invention relates to the use of any of a combination of an analog, derivative, isomer, metabolite, pharmaceutically acceptable salt, pharmaceutical product, hydrate, or N-oxide of the anti-cancer compounds of the present invention.

As defined herein, the term “isomer” includes, but is not limited to, optical isomers and analogs, structural isomers and analogs, conformational isomers and analogs, and the like.

In one embodiment, this invention encompasses the use of various optical isomers of the anti-cancer compound. It will be appreciated by those skilled in the art that the anti-cancer compounds of the present invention contain at least one chiral center. Accordingly, the anti-cancer compounds used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mutures thereof, which form possesses properties useful in the treatment of cancer as described herein. In one embodiment, the anti-cancer compounds are the pure (R)-isomers. In another embodiment, the anti-cancer compounds are the pure (S)-isomers. In another embodiment, the anti-cancer compounds are a mixture of the (R) and the (S) isomers. In another embodiment, the anti-cancer compounds are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes derivatives of the anti-cancer compounds. The term “derivatives” includes but is not limited to ether derivatives, acid derivatives, amide derivatives, ester derivatives and the like. In addition, this invention further includes hydrates of the anti-cancer compounds. The term “hydrate” includes but is not limited to hemihydrate, monohydrate, dihydrate, trihydrate and the like.

This invention further includes metabolites of the anti-cancer compounds. The term “metabolite” means any substance produced from another substance by metabolism or a metabolic process.

This invention further includes pharmaceutical products of the anti-cancer compounds. The term “pharmaceutical product” means a composition suitable for pharmaceutical use (pharmaceutical composition), as defined herein.

This invention further includes prodrugs of the anti-cancer compounds. The term “prodrug” means a substance which can be converted in-vivo into a biologically active agent by such reactions as hydrolysis, esterification, desterification, activation, salt formation and the like.

This invention further includes crystals of the anti-cancer compounds. Furthermore, this invention provides polymorphs of the anti-cancer compounds. The term “crystal” means a substance in a crystalline state. The term “polymorph” refers to a particular crystalline state of a substance, having particular physical properties such as X-ray diffraction, IR spectra, melting point, and the like.

In another embodiment, the present invention provides a process for preparing the anti-cancer compounds of the present invention.

The process of the present invention is suitable for large-scale preparation, since all of the steps give rise to highly pure compounds, thus avoiding complicated purification procedures that ultimately lower the yield. Thus the present invention provides methods for the synthesis of non-steroidal agonist compounds that can be used for industrial large-scale synthesis and that provide highly pure products in high yield.

Thus, in another embodiment, the present invention provides process for preparing an anti-cancer compound represented by the structure of formula I: 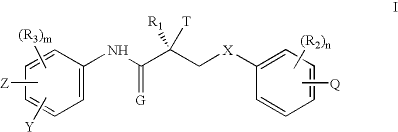
wherein
X is a bond, O, CH₂, NH, S, SO, SO₂, Se, PR, NO or NR;
G is O or S;
T is OH, OR, —NHCOCH₃, NHCOR or 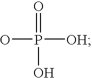
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 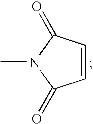
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 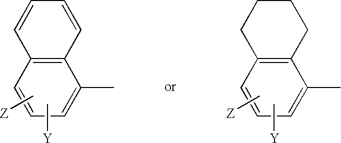
n is an integer of 1-4; and
m is an integer of 1-3;
the process comprising the step of coupling a compound of formula VIII: 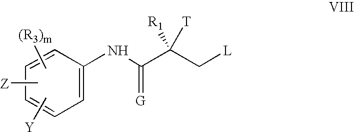
   wherein Z, Y, G, RI, T, R₃ and m are as defined above and L is a leaving group, with a compound of formula IX: 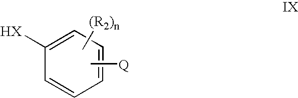
   wherein Q, X, R₂ and n are as defined above.

In one embodiment of the invention, in the anti-cancer compound represented by the structure of formula I, if Z is NO₂, CN, COR, COOH or CONHR, Q is not NHCOCH₂A or N₃;

In another embodiment, the present invention provides process for preparing an anti-cancer compound represented by the structure of formula I: 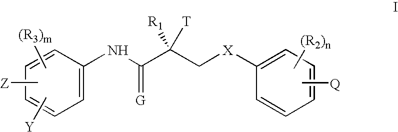
wherein
X is SO or SO₂;
G is O or S;
T is OH, OR, —NHCOCH₃, NHCOR, or 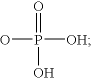
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 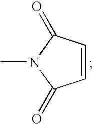
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONF, CF₃ or SnR₃, or R₃ together with the benzene ring to which it is attached forms a fused ring system represented by the structure: 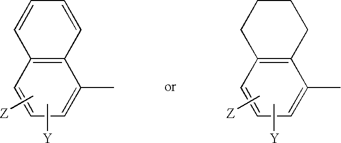
n is an integer of 1-4; and
m is an integer of 1-3;
the process comprising the step of coupling a compound of formula VIII: 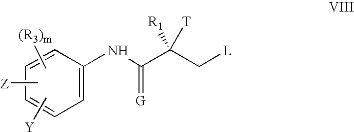
   wherein Z, Y, G, R₁, T, R₃ and m are as defined above and L is a leaving group, with a compound of formula IX: 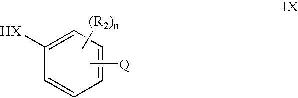
   wherein Q, X R₂ and n are as defined above.

In one embodiment, the coupling step is carried out in the presence of a base. In another embodiment, the leaving group L is Br. In another embodiment, the compound of formula VIII is prepared by
a. preparing a compound of formula X by ring opening of a cyclic compound of formula XI 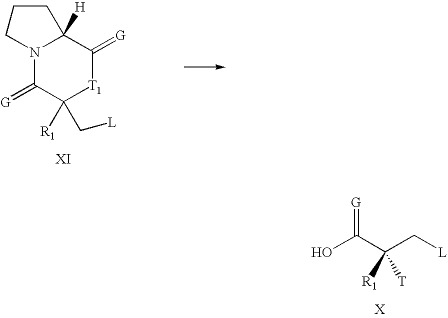
   wherein L, R₁, G and T are as defined above, and T₁ is O or NH; and
b. reacting an amine of formula XII: 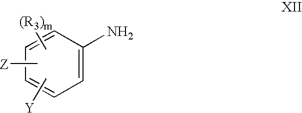
   wherein Z, Y, R₃ and m are as defined above, with the compound of formula X, in the presence of a coupling reagent, to produce the compound of formula VIII.

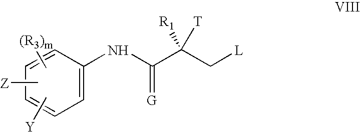

In one embodiment, step (a) is carried out in the presence of HBr. In another embodiment, the process further comprises the step of converting the anti-cancer compound to its analog, isomer, metabolite, derivative, pharmaceutically acceptable salt, pharmaceutical product, N-oxide, hydrate or any combination thereof.

In another embodiment, the present invention provides process for preparing an anti-cancer compound represented by the structure of formula II: 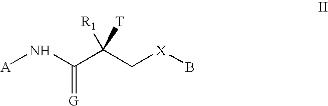
wherein
X is a bond, O, CH₂, NH, S, SO, SO₂, Se, PR, NO or NR;
G is O or S;
T is OH, OR, —NHCOCH₃, NHCOR or 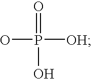
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 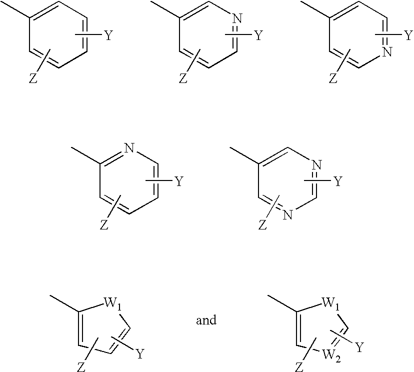
B is a ring selected from: 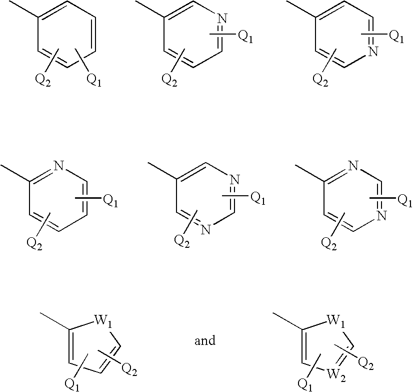
   wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 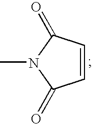
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 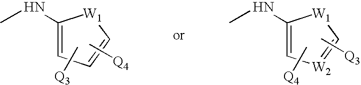
Q³ and Q⁴ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;
   the process comprising the step of coupling a compound of formula XIII: 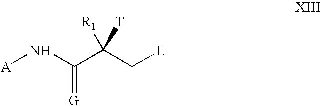
   wherein A, G, R₁ and T are as defined above and L is a leaving group, with a compound of formula HX-B wherein B and X are as defined above.

In one embodiment of the invention, in the anti-cancer compound represented by the structure of formula II, if Z is NO₂, CN, COR, COOH or CONHR, Q is not NHCOCH₂A or N₃;

In another embodiment, the present invention provides process for preparing an anti-cancer compound represented by the structure of formula II: 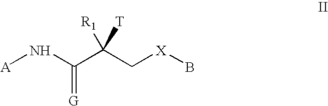
wherein
X is SO or SO₂;
G is O or S;
T is OH, OR, —NHCOCH₃, NHCOR or 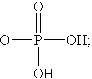
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 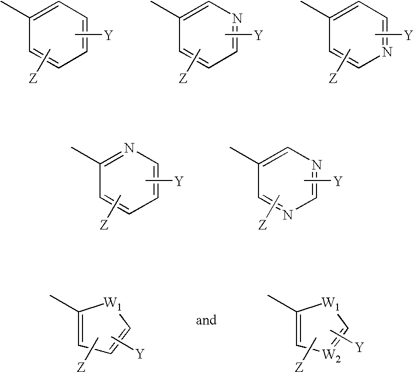
B is a ring selected from: 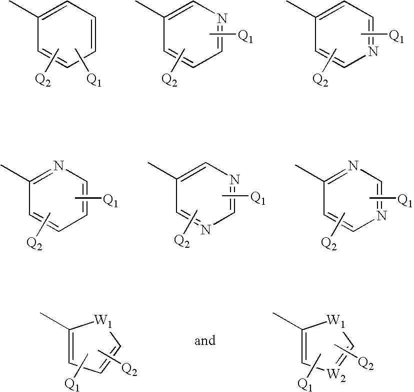
   wherein A and B cannot simultaneously be a benzene ring;
Y is CF₃, F, I, Br, Cl, CN CR₃ or SnR₃;
one of Z or Q, is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 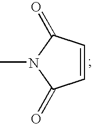
A is F, Cl, Br or I
Q² is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHFSO₂CH₃, NHSO₂R, OR, COR, OCOR, OSO₂R, SO₂R, SR, 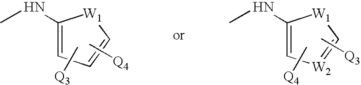
Q³ and Q⁴ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O,NH,NR,NO or S; and
W₂ is N or NO;
   the process comprising the step of coupling a compound of formula XIII: 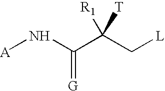
   wherein A, G, R, and T are as defined above and L is a leaving group,
with a compound of formula HX-B wherein B and X are as defined above.

In one embodiment of the invention, in the anti-cancer compound represented by the structure of formula II, if Z is NO₂, CN, COR, COOH or CONHR, Q is not NHCOCH₂A or N₃;

In one embodiment, the coupling step is carried out in the presence of a base. In another embodiment, the leaving group L is Br. In another embodiment, the compound of formula XIII is prepared by
a. preparing a compound formula X by ring opening of a cyclic compound of formula XI 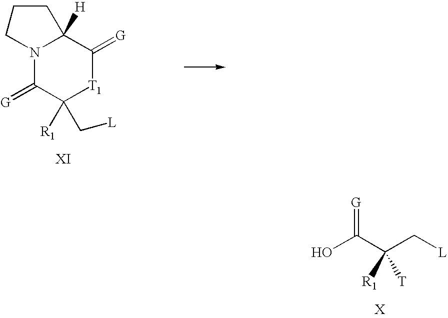
   wherein L, R₁, G and T are as defined above, and T₁ is O or NH; and
b. reacting an amine of formula A-NH₂ wherein A is as defined above, with the compound of formula X in the presence of a coupling reagent, to produce the amide of formula XIII.

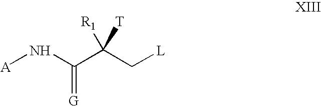

In one embodiment, step (a) is carried out in the presence of HBr. In another embodiment, the process further comprises the step of converting the anti-cancer compound to its analog, isomer, metabolite, derivative, pharmaceutically acceptable salt, pharmaceutical product, N-oxide, hydrate or any combination thereof.

In another embodiment, the present invention provides process for preparing an anti-cancer compound represented by the structure of formula III: 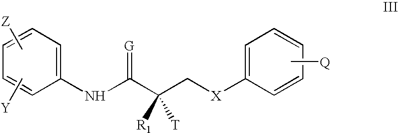
wherein
X is a bond, O, CH₂, NH, S, SO, SO₂, Se, PR, NO or NR;
G is O or S;
T is OH, OR, —NHCOCH₃, NHCOR or 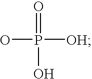
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 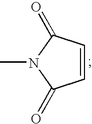
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃;
   the process comprising the step of coupling a compound of formula XIV: 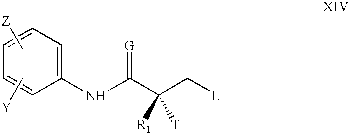
   wherein Z, Y, G R₁ and T are as defmed above and L is a leaving group, with a compound of formula XV: 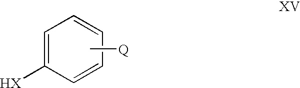
   wherein Q and X are as defined above.

In one embodiment of the invention, in the anti-cancer compound represented by the structure of formula III, if Z is NO₂, CN, COR, COOH or CONHR, Q is not NHCOCH₂A or N₃;

In another embodiment, the present invention provides process for preparing an anti-cancer compound represented by the structure of formula III: 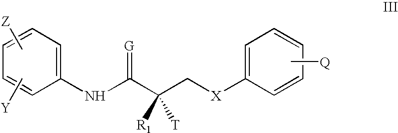
wherein
X is SO or SO₂;
G is O or S;
T is OH, OR, —NHCOCH₃, NHCOR, or 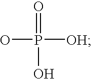
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 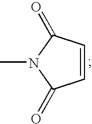
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃;
   the process comprising the step of coupling a compound of formula XIV: 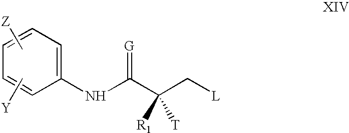
   wherein Z, Y, G R₁ and T are as defined above and L is a leaving group, with a compound of formula XV: 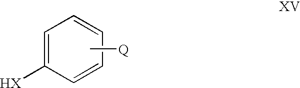
   wherein Q and X are as defined above.

In one embodiment, the coupling step is carried out in the presence of a base. In another embodiment, the leaving group L is Br. In another embodiment, the compound of formula XWV is prepared by
a. preparing a compound formula X by ring opening of a cyclic compound of formula XI 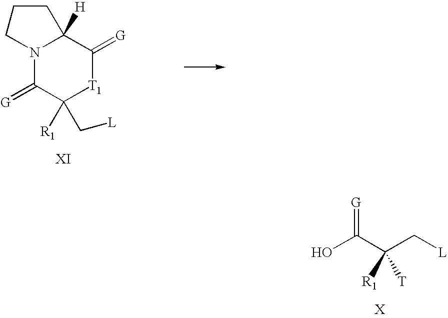
   wherein L, R₁, and T are as defined above, G is O and T₁ is O or NH; and
b. reacting an amine of formula XVI 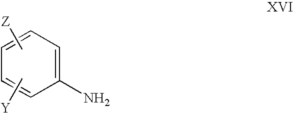
   with the compound of formula X in the presence of a coupling reagent, to produce the compound of formula XIV.

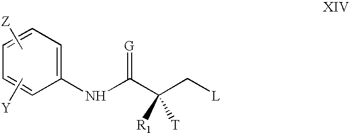

In one embodiment, step (a) is carried out in the presence of HBr. In another embodiment, the process further comprises the step of converting the anti-cancer compound to its analog, isomer, metabolite, derivative, pharmaceutically acceptable salt, pharmaceutical product, N-oxide, hydrate or any combination thereof.

In another embodiment, the present invention provides process for preparing an anti-cancer compound represented by the structure of formula IV: 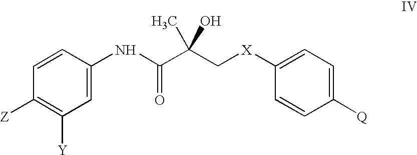
wherein
X is a bond, O, CH₂, NH, S, SO, SO₂, Se, PR, NO or NR;
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH2A or 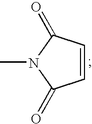
A is F, Cl, Br or I; and
R is alkyl, haloalkyl, dihaloalkyl, trihaloalkyl, CH₂F, CHF₂, CF₃, CF₂CF₃, aryl, phenyl, halogen, alkenyl or OH;
   the process comprising the step of coupling an amide of formula XVII: 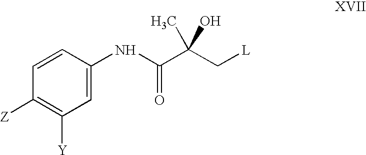
   wherein Z and Y are as defined above and L is a leaving group, with a compound of formula XVIII: 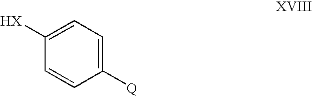
   wherein Q and X R₂ are as defined above.

In one embodiment of the invention, in the anti-cancer compound represented by the structure of formula I, if Z is NO₂, CN, COR, COOH or CONHR, Q is not NHCOCH₂A or N₃;

In another embodiment, the present invention provides process for preparing an anti-cancer compound represented by the structure of formula IV: 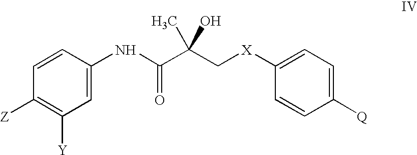
wherein
X is SO or SO₂;
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 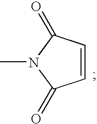
A is F, Cl, Br or I; and
R is alkyl, haloalkyl, dihaloalkyl, trihaloalkyl, CH₂F, CHF₂, CF₃, CF₂CF₃, aryl, phenyl, halogen, alkenyl or OH;
   the process comprising the step of coupling an amide of formula XVII: 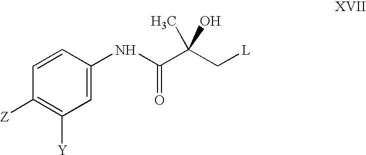
   wherein Z and Y are as defined above and L is a leaving group, with a compound of formula XVIII: 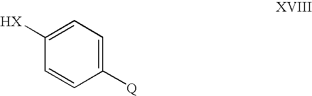
   wherein Q and X R₂ are as defined above.

In one embodiment, the coupling step is carried out in the presence of a base. In another embodiment, the leaving group L is Br. In another embodiment, the compound of formula XVII is prepared by
a. preparing a compound formula X by ring opening of a cyclic compound of formula XI 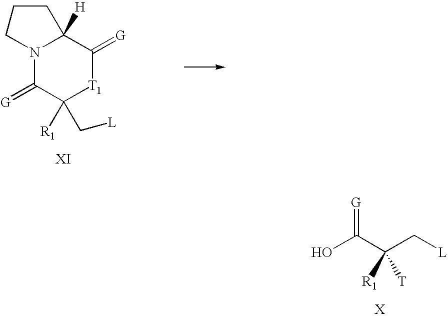
   wherein L, R₁, and T are as defined above, G is O and T₁ is O or NH; and
b. reacting an amine of formula XVIX 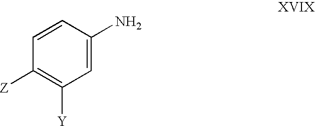
   with the compound of formula X in the presence of a coupling reagent, to produce the compound of formula XVII.

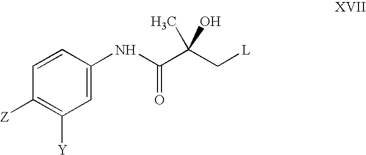

In one embodiment, step (a) is carried out in the presence of HBr. In another embodiment, the process further comprises the step of purifying the anti-cancer compound using a mixture of ethanol and water. In another embodiment, the process further comprises the step of converting the anti-cancer compound to its analog, isomer, metabolite, derivative, pharmaceutically acceptable salt, pharmaceutical product, N-oxide, hydrate or any combination thereof

In another embodiment, the present invention provides process for preparing an anti-cancer compound represented by the structure of the formula XXI: 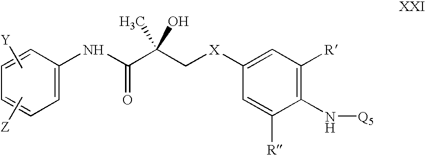
wherein
X is O, S, or SO;
Y is CF₃, F, Cl, Br, I, CN, or SnR₃;
Z is NO₂, CN, COR, COOH, CONE, F, Cl, Br or I;
Q₅ is COCH═CH₂ or COCH₂A;
A is F, Cl, Br or I; and
each of R′ and R″, independently, are H, F, Cl, Br, I or alkyl.
   the process comprising the steps of:
coupling a compound represented by the structure of the formula XXVII: 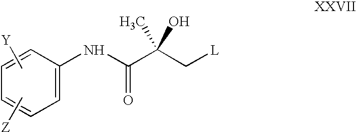
   wherein Y and Z are defined above and L is a leaving group, with a compound represented by the structure of the formula XXVIII: 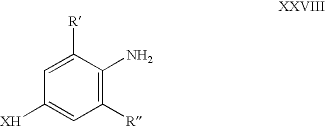
   wherein R′, R″ and X are defined above, to prepare a compound represented by the structure of the formula XXIX: 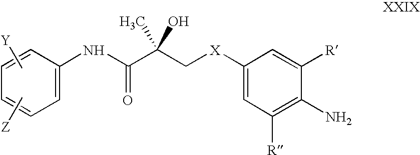
   wherein R′, R″, X, Y and Z are defined above; and reacting the compound formula XXIX with A-Q₅, wherein A and Q₅ are defined above, is to obtain a compound represented by the structure of the formula XXI.

In another embodiment, the present invention provides process for preparing an anti-cancer compound represented by the structure of the formula XXIII: 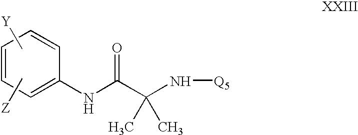
wherein
Y is CF₃, F, Cl, Br, I, CN, or SnR₃;
Z is NO₂, CN, COR, COOH, CONHR, F, Cl, Br or I;
Q₅ is COCH═CH₂ or COCH₂A; and
A is F, Cl, Br or I,
the process comprising the step of:
reacting a compound represented by the structure of the formula XXX: 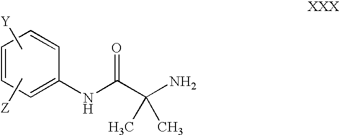
   wherein Y and Z are defined above,
with A-Q₅, wherein A and Q₅ are defined above, to obtain a compound represented by the structure of the formula XXII.

In another embodiment, the present invention provides process for preparing an anti-cancer compound represented by the structure of the formula XXV: 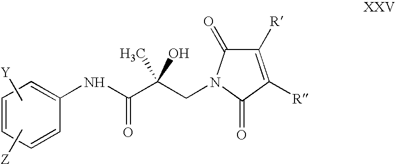
wherein
Y is CF₃, F, Cl, Br, I, CN, or SnR₃;
Z is NO₂, CN, COR, COOH, CONHR, F, Cl, Br or I; and
R′ and R″ are, independently of each other, a hydrogen, alkyl or a halogen,
the process comprising the step of:
reacting a compound represented by the structure of the formula XXXI: 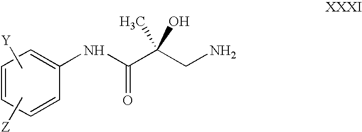
   wherein Y and Z are defined above, with a an unsubstituted or a substituted phthalic anhydride represented by the structure of the formula XXXII: 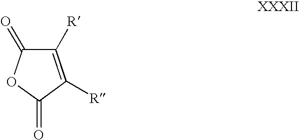
   wherein R′ and R″ are defined above, to obtain a compound represented by the structure of the formula XXV.

As demonstrated herein, Applicants have found that when the purification step of the anti-cancer compounds is carried out in the presence of a nontoxic organic solvent and water, such as ethanol and water, for example by recrystallization from a mixture of ethanol and water, a highly pure product with excellent crystal stability is obtained in high yields. In addition, the use of a nontoxic organic solvent/water for purification is safe and cheap, and avoids any biological hazards that may arise from the use of toxic organic solvents such as hexane. In one embodiment, the nontoxic organic solvent is ethanol.

Thus, in one embodiment, the present invention provides a synthetic process for preparing the anti-cancer compounds described herein, which involves a purification step comprising crystallization of the anti-cancer product using a mixture of a nontoxic organic solvent and water. In one embodiment, the nontoxic organic solvent is ethanol. In a particular embodiment, the crystallization step comprises mixing an ethanol solution comprising the anti-cancer compound with water, so as to crystallize the anti-cancer compound. In a further embodiment, the process further comprises the step of collecting the anti-cancer compound by filtration.

The process of the present invention is suitable for large-scale preparation, since all of the steps give rise to highly pure compounds, thus avoiding complicated purification procedures that ultimately lower the yield. Thus the present invention provides methods for the synthesis of the anti-cancer compounds of the present invention that can be used for industrial large-scale synthesis and that provide highly pure products in high yield. In addition, the methods described by the present invention utilize safe, environmentally friendly and cheap reagents and purification steps, thus avoiding any undesirable toxicological issues that may arise from the use of toxic, environmentally unfriendly or biologically unstable reagents.

It should be apparent to a person skilled in the art that any nontoxic organic solvent is suitable in the methods of the present invention, for example alcohols such as methanol or ethanol, aromatic compounds such as toluene and xylene, DMSO, THF, cyclohexane and the like.

In one embodiment, the nontoxic organic solvent is ethanol. Any grade and purity level of ethanol is suitable. In one embodiment, the ethanol is neat ethanol. In another embodiment, the ethanol is an ethanol solution that contains denaturants, such as toluene, methanol and the like.

It is understood by a person skilled in the art that when T₁ is O or NH, T is compound VIII is O or NH₂. Thus, when T in compound I is OR, the reaction will involve a further step of converting the OH to OR by a reaction with, for example, an alkyl halide R—X. When T in compound I is NHCOR, NHCOCH₃, the reaction will involve a further step of converting the NH₂ to NHCOR or NHCOCH₃, by a reaction with, for example, the corresponding acyl chloride ClCOR or ClCOCH₃.

In one embodiment, the coupling step defined hereinabove is carried out in the presence of a base. Any suitable base that will deprotonate the hydrogen of the —XH moiety (for example, a phenol moiety when X is O) and allow the coupling may be used. Nonlimiting examples of bases are carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), alkali metal hydrides such as sodium hydride (NaH), potassium hydride (KH) and lithium hydride (LiH), and the like.

The leaving group L is defined herein as any removable group customarily considered for chemical reactions, as will be known to the person skilled in the art. Suitable leaving groups are halogens, for example F, Cl, Br and I; alkyl sulfonate esters (—OSO₂R) wherein R is an alkyl group, for example methanesulfonate (mesylate), trifluoromethanesulfonate, ethanesulfonate, 2,2,2-trifiuoroethanesulfonate, perfluoro butanesulfonate; aryl sulfonate esters (—OSO₂Ar) wherein Ar is an aryl group, for example p-toluoylsulfonate (tosylate), benzenesulphonate which may be unsubstituted or substituted by methyl, chlorine, bromine, nitro and the like; NO₃, NO₂, or sulfate, sulfite, phosphate, phosphite, carboxylate, imino ester, N₂ or carbamate.

The reaction is conveniently carried out in a suitable inert solvent or diluent such as, for example, tetrahydrofuran, diethyl ether, aromatic amines such as pyridine; aliphatic and aromatic hydrocarbons such as benzene, toluene, and xylene; dimethylsulfoxide (DMSO), dimethylformamide (DMF), and dimethylacetamide (DMAC). The reaction is suitably carried out at a temperature in the range of, for example, −20 to 120 C., for example at or near ambient temperature.

The coupling reagent defined hereinabove is a reagent capable of turning the carboxylic acid/thiocarboxylic acid of formula X into a reactive derivative thereof, thus enabling coupling with the respective amine amine to form an amide/thioamide bond. A suitable reactive derivative of a carboxylic acid/thiocarboxylic acid is, for example, an acyl halide/thioacyl halide, for example an acyl/thioacyl chloride formed by the reaction of the acid/thioacid and an inorganic acid chloride, for example thionyl chloride; a mixed anhydride, for example an anhydride formed by the reaction of the acid and a chloroformate such as isobutyl chloroformate; an active ester/thioester, for example an ester/thioester formed by the reaction of the acid/thioacid and a phenol, an ester/thioester or an alcohol such as methanol, ethanol, isopropanol, butanol or N-hydroxybenzotriazole; an acyl/thioacyl azide, for example an azide formed by the reaction of the acid/thioacid and azide such as diphenylphosphoryl azide; an acyl cyanide/thioacyl cyanide, for example a cyanide formed by the reaction of an acid and a cyanide such as diethylphosphoryl cyanide; or the product of the reaction of the acid/thioacid and a carbodiimide such as dicyclohexylcarbodiimide.

The reaction is conveniently carried out in a suitable inert solvent or diluent as described hereinabove, suitably in the presence of a base such as triethylamine, and at a temperature in the range, as described above.
In another embodiment of the invention, there is provided a compound according to the structure as set forth in Formula 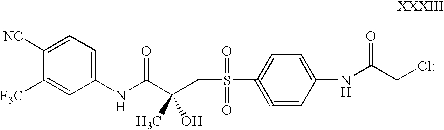
In another embodiment of the invention, there is provided a compund according to the structure set forth in formula XXXIV: 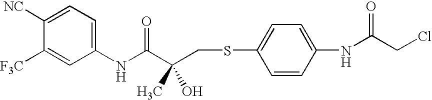
In another embodiment, there is provided a compund according to the structure set forth in formula XXXV: 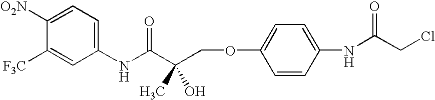
In another embodiment, there is provided a compund according to the structure set forth in formula XXXVI:

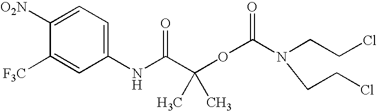

In another embodiment of the invention, there is provided a compound according to the following formula:

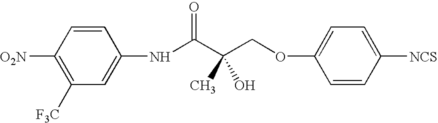

In another embodiment of the invention, there is provided a compound according to the following formula:

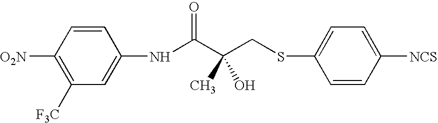

In an embodiment of the invention, there is provided an anti-cancer compound represented by the structure of formula I: 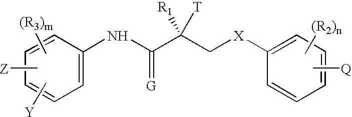
X is a bond, O, CH₂, NH, S, SO, SO₂, Se, PR, NO or NR;
G is O or S;
T is OH, OR, —NHCOCH₃, NHCOR or 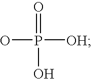
Y is CF₃, F, Cl, Br, I, CN, or SnR₃;
Z and Q are same or different and are NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, NHCOCF₃, NHCOCH₂OH, NHCOCH₃, N═C═NH, COCH₂A or 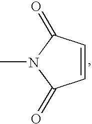
   with the provisio that when Z is NO₂, CN, COR, COOH or CONHR, Q is not NHCOCH₂A or N₃;
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are H, F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are H, F, Cl, Br, I, CN, NO₂, COR, COOH, CONHR, CF₃ or SnR₃, or R₃ together with the benzene ring to which it is attached forms a fused ring system represented by the structure: 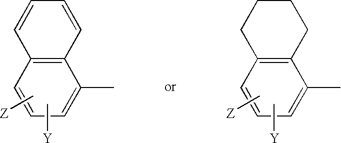
n is an integer of 1-4; and
m is an integer of 1-3.

In an embodiment of the invention there is provided a compound according to the following formula:

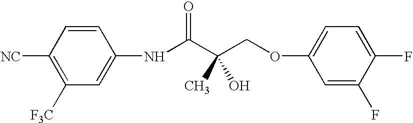

In another embodiment of the invention there is provided a compound according to the following formula:

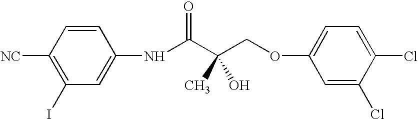

In another embodiment of the invention there is provided a compound according to the following formula:

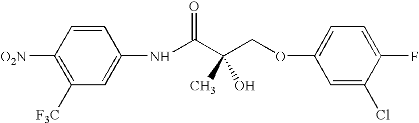

In another embodiment of the invention there is provided a compound according to the following formula:

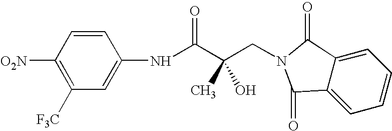

In another embodiment of the invention there is provided a compound according to the following formula:

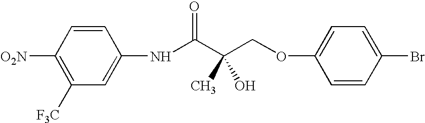

In another embodiment of the invention there is provided a compound according to the following formula:

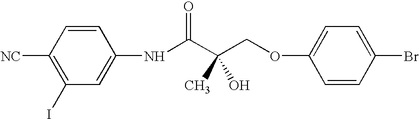

In another embodiment of the invention there is provided a compound according to the following formula:

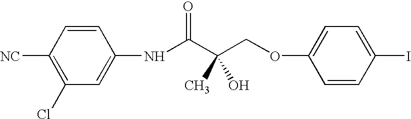

In another embodiment of the invention there is provided a compound according to the following formula:

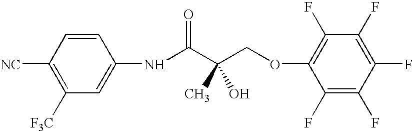

In another embodiment of the invention there is provided a compound according to the following formula:

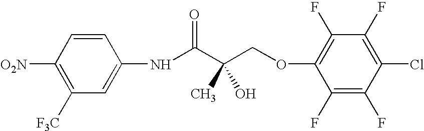

In another embodiment of the invention there is provided a compound according to the following formula:

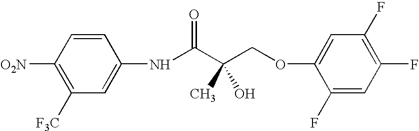

In another embodiment of the invention there is provided a compound according to the following formula:

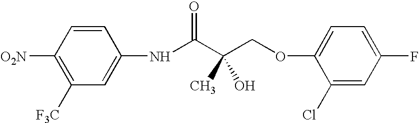

In another embodiment of the invention there is provided a compound according to the following formula:

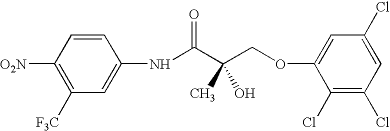

In another embodiment of the invention there is provided a compound according to the following formula:

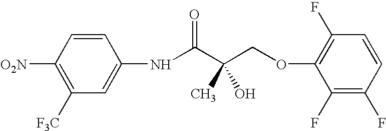

In another embodiment of the invention there is provided a compound according to the following formula:

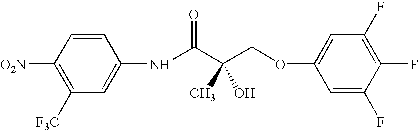

In another embodiment of the invention there is provided a compound according to the following formula:

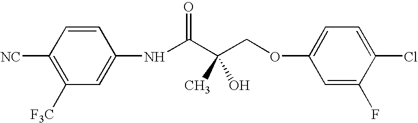

In another embodiment of the invention there is provided a compound according to the following formula:

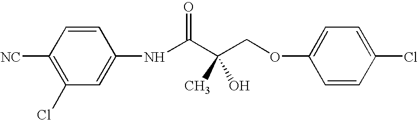

In another embodiment of the invention there is provided a compound according to the following formula:

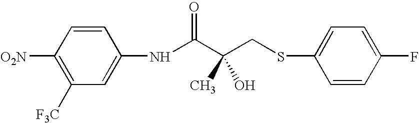

In another embodiment of the invention there is provided a compound according to the following formula:

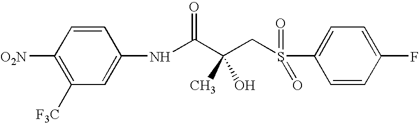

In another embodiment of the invention there is provided a compound according to the following formula:

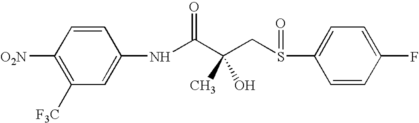

In another embodiment of the invention there is provided a compound according to the following formula:

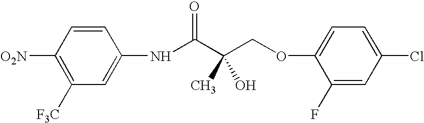

In another embodiment of the invention there is provided a compound according to the following formula:

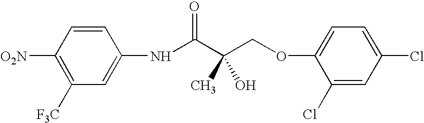

In another embodiment of the invention there is provided a compound according to the following formula:

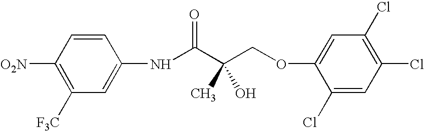

In another embodiment of the invention there is provided a compound according to the following formula:

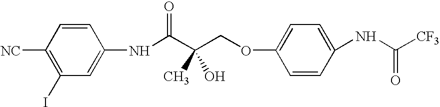

In another embodiment of the invention there is provided a compound according to the following formula:

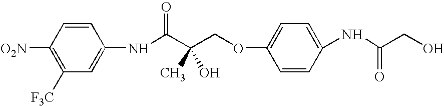

In another embodiment of the invention there is provided a compound according to the following formula:

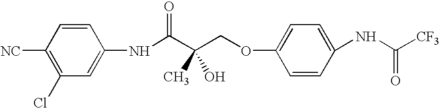

In another embodiment of the invention there is provided a compound according to the following formula:

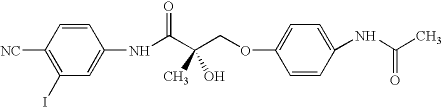

In another embodiment of the invention there is provided a compound according to the following formula:

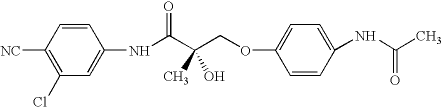

In another embodiment of the invention there is provided a compound according to the following formula:

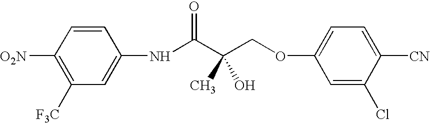

In another embodiment of the invention there is provided a compound according to the following formula:

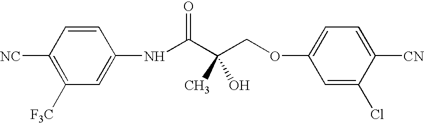

In another embodiment of the invention there is provided a compound according to the following formula:

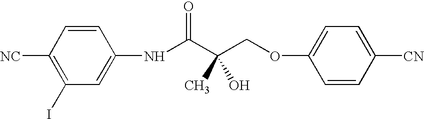

In another embodiment of the invention there is provided a compound according to the following formula:

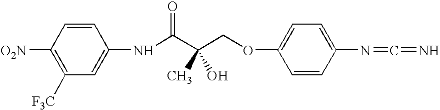

In another embodiment of the invention there is provided a compound according to the following formula:

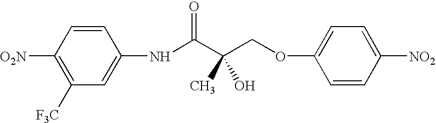

In another embodiment of the invention there is provided a compound according to the following formula:

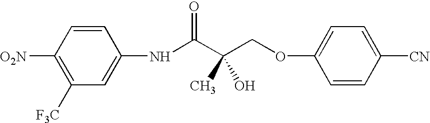

In another embodiment of the invention there is provided a compound according to the following formula:

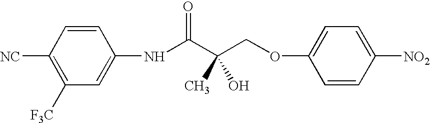

In another embodiment of the invention there is provided a compound according to the following formula:

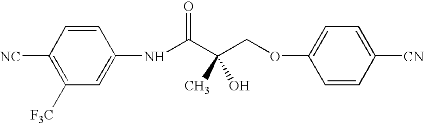

In another embodiment of the invention there is provided a compound according to the following formula:

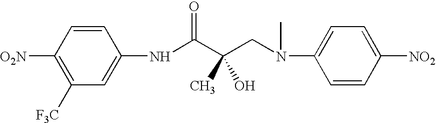

In another embodiment of the invention there is provided a compound according to the following formula:

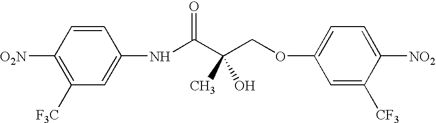

In another embodiment of the invention there is provided a compound according to he following formula:

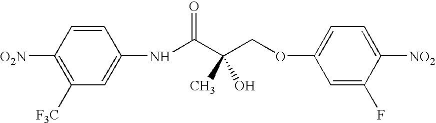

In another embodiment of the invention there is provided a compound according to the following formula:

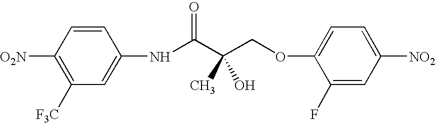

In another embodiment of the invention there is provided a compound according to the following formula:

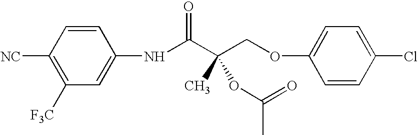

In another embodiment of the inevtnion, there is provided an anti-cancer compound represented by the structure of formula II: 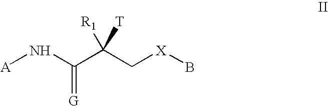
wherein
X is a bond, O, CH₂, NH, S, SO, SO₂, Se, PR, NO or NR;
G is O or S; 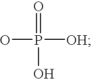
T is OH, OR, —NHCOCH₃, NHCOR or
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;

A is a ring selected from:

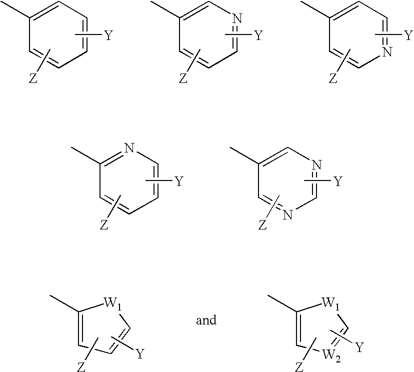

B is a ring selected from: 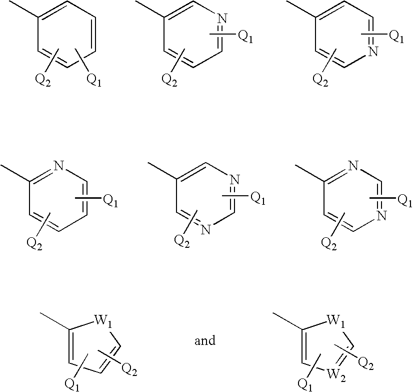
wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂,
COCH₂A or 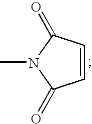
   wherein if Z is CN, Q₁ is hydrogen;
with the provisio that when Z is NO₂, CN, COR, COOH or CONHR, Q is not NHCOCH₂A or N₃;
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 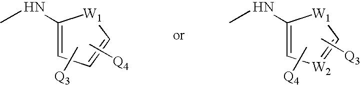
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;
or its analog, isomer, metabolite, derivative, pharmaceutically acceptable salt, pharmaceutical product, N-oxide, hydrate or any combination thereof.

In an embodiment of the invention there is provided a compound according to the following formula:

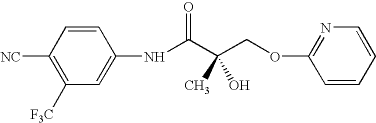

In another embodiment of the invention there is provided a compound according to the following formula: 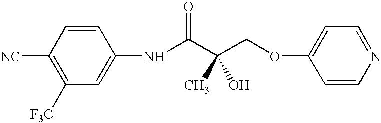
Biological Effects of the Anti-Cancer Agents

As contemplated herein, the compounds of the present invention are effective, either alone or in a composition, are useful for treating cancer, preventing cancer, delaying the progression of cancer, treating and/or preventing the recurrence of cancer, suppressing, inhibiting or reducing the incidence of cancer, or inducing apoptosis in a cancer cell.

Thus, in one embodiment, the present invention further provides a method of treating a subject suffering from cancer, comprising the step of administering to the subject the anti-cancer compound of any of formulae I-IV, XIX-XXVI, XIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to treat cancer in the subject.

In another embodiment, the present invention provides a method of preventing cancer in a subject, comprising the step of administering to the subject the anti-cancer compound of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to prevent cancer in the subject.

In another embodiment, the present invention further provides a method of delaying the progression of cancer in a subject suffering from cancer, comprising the step of administering to the subject the anti-cancer compound of any of any of formulae is I-IV, XIX-XXVI, XIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to delay the progression of cancer in the subject.

In another embodiment, the present invention further provides a method of preventing the recurrence of cancer in a subject suffering from cancer, comprising the step of administering to the subject the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to prevent the recurrence of cancer in the subject.

In another embodiment, the present invention provides a method of treating the recurrence of cancer in a subject suffering from cancer, comprising the step of administering to the subject the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to treat the recurrence of cancer in the subject.

In another embodiment, the present invention provides a method of suppressing, inhibiting or reducing the incidence of cancer in a subject suffering from cancer, comprising the step of administering to the subject the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to suppress, inhibit or reduce the incidence of cancer in the subject.

In another embodiment, the present invention further provides a method of irreversibly binding an anti-cancer compound to a cellular component, comprising the step of contacting a cell comprising the cellular component with the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to irreversibly bind the anti-cancer compound to the cellular component.

In another embodiment, the present invention further provides a method of alkylating a cellular component, comprising the step of contacting a cell comprising the cellular component with the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to alkylate the cellular component.

As used herein, the term “cancer” is interchangeable with the terms malignancy, malignant or neoplasm, and refers to a disease of cells characterized by an abnormal growth of cells that tend to proliferate in an uncontrolled way and, in some cases, to metastasize. Cancer is a disorder in which a population of cells has become, in varying degrees, unresponsive to the control mechanisms that normally govern proliferation and differentiation. Cancer refers to various types of malignant neoplasms and tumors, including metastasis to different sites.

Nonlimiting examples of cancers that can be treated with the anti-cancer compounds of the present invention are adenocarcinoma, adrenal gland tumor, ameloblastoma, anaplastic tumor, anaplastic carcinoma of the thyroid cell, angiofibroma, angioma, angiosarcoma, apudoma, argentaffinoma, arrhenoblastoma, ascites tumor cell, ascitic tumor, astroblastoma, astrocytoma, ataxia-telangiectasia, atrial myxoma, basal cell carcinoma, benign tumor, bone cancer, bone tumor, brainstem glioma, brain tumor, breast cancer, Burkitt's lymphoma, carcinoma, cerebellar astrocytoma, cervical cancer, cherry angioma, cholangiocarcinoma, a cholangioma, chondroblastoma, chondroma, chondrosarcoma, chorioblastoma, choriocarcinoma, colon cancer, common acute lymphoblastic leukaemia, craniopharyngioma, cystocarcinoma, cystofibroma, cystoma, cytoma, ductal carcinoma in situ, ductal papilloma, dysgerminoma, encephaloma, endometrial carcinoma, endothelioma, ependymoma, epithelioma, erythroleukaemia, Ewing's sarcoma, extra nodal lymphoma, feline sarcoma, fibroadenoma, fibrosarcoma, follicular cancer of the thyroid, ganglioglioma, gastrinoma, glioblastoma multiforme, glioma, gonadoblastoma, haemangioblastoma, haemangioendothelioblastoma, haemangioendothelioma, haemangiopericytoma, haematolymphangioma, haemocytoblastoma, haemocytoma, hairy cell leukaemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angiofibroma, Kaposi sarcoma, kidney tumour, large cell lymphoma, leukemia, chronic leukemia, acute leukemia, lipoma, liver cancer, liver metastases, Lucke carcinoma, lymphadenoma, lymphangioma, lymphocytic leukaemia, lymphocytic lymphoma, lymphocytoma, lymphoedema, lymphoma, lung cancer, malignant mesothelioma, malignant teratoma, mastocytoma, medulloblastoma, melanoma, meningioma, mesothelioma, metastatic cancer, Morton's neuroma, multiple myeloma, myeloblastoma, myeloid leukemia, myelolipoma, myeloma, myoblastoma, myxoma, nasopharyngeal carcinoma, nephroblastoma, neuroblastoma, neurofibroma, neurofibromatosis, neuroglioma, neuroma, non-Hodgkin's lymphoma, oligodendroglioma, optic glioma, osteochondroma, osteogenic sarcoma, osteosarcoma, ovarian cancer, Paget's disease of the nipple, pancoast tumor, pancreatic cancer, phaeochromocytoma, pheochromocytoma, plasmacytoma, primary brain tumor, progonoma, prolactinoma, prostate cancer, prostate carcinogenesis, pre-malignant lesions of prostate cancer, prostate intraepithelial neoplasia (PIN), high prostate intraepithelial neoplasia (HPIN), renal cell carcinoma, retinoblastoma, rhabdomyosarcoma, rhabdosarcoma, solid tumor, sarcoma, secondary tumor, seminoma, skin cancer, small cell carcinoma, squamous cell carcinoma, strawberry haemangioma, T-cell lymphoma, teratoma, testicular cancer, thymoma, trophoblastic tumor, tumourigenic, vestibular schwannoma, Wilm's tumor, or a combination thereof.

A “cancer cell” is defined herein as a neoplastic cell, a pre-malignant cell, a metastatic cell, a malignant cell, a tumor cell, an oncogenic cell, a cell with a cancer genotype, a cell of malignant phenotype, a cell with a malignant genotype, a cell displaying cancer-associated metabolic atypia, an oncogene transfected cell, a virus-transformed cell, a cell that expresses a marker for an oncogene, a cell that expresses a marker for cancer, or a combination thereof.

The compounds of the present invention contain a functional group (e.g. haloacetamide or azide), which promotes irreversible binding to biological targets, i.e. covalent bond formation with cellular components. Thus, in one embodiment, the compounds are alkylating agents, which bind irreversibly to biological targets such as nucleic acids and proteins.

An “alkylating agent” is defined herein as an agent that alkylates (forms a covalent bond) with a cellular component, such as protein, DNA, RNA or enzyme. It is a highly reactive chemical that introduces alkyl radicals into biologically active molecules and thereby prevents their proper functioning. The alkylating moiety is an electrophilic group that interacts with nucleophilic moieties in cellular components. For example, in one embodiment, an alkylating group is an isocyanate moiety, an electrophilic group that forms covalent bonds with nucleophilic groups (N, O, S etc.) in cellular components. In another embodiment, an alkylating group is an isothiocyanate moiety, another electrophilic group that forms covalent bonds with nucleophilic groups (N, O, S etc.) in cellular components. In another embodiment, an alkylating group is a haloalkyl (CH₂Hal wherein Hal is halogen), an electrophilic group that forms covalent bonds with nucleophilic groups in cellular components. In another embodiment, an alkylating group is a haloalkyl-amido (NHCOCH₂X wherein X is halogen), an electrophilic group that forms covalent bonds with nucleophilic groups in cellular components. In another embodiment, the alkylating group is an azide, also an electrophilic group that forms covalent bonds with nucleophilic groups in cellular components.

A “cellular component” is defined herein as any intracellular, extracellular, or membrane bound component found in a cell.

In another embodiment, the present invention provides a method of inducing apoptosis in a cancer cell, comprising the step of contacting the cell with the anti-cancer compound of any of any of formulae I-IV, XIX-XXVI, XXXIII-XXXIV and any other compund described herein and/or its analog, derivative, isomer, metabolite, pharmaceutically acceptable salt, pharmaceutical product, hydrate, N-oxide, impurity, prodrug, polymorph, crystal, or any combination thereof, in an amount effective to induce apoptosis in the cancer cell.

As defined herein, “apoptosis”, or programmed cell death, is a form of cell death in which a programmed sequence of events leads to the elimination of cells without releasing harmful substances into the surrounding area. Apoptosis plays a crucial role in developing and maintaining health by eliminating old cells, unnecessary cells, and unhealthy cells.

As defined herein, “contacting” means that the anti-cancer compound of the present invention is introduced into a sample containing the enzyme in a test tube, flask, tissue culture, chip, array, plate, microplate, capillary, or the like, and incubated at a temperature and time sufficient to permit binding of the anti-cancer compound to the enzyme. Methods for contacting the samples with the anti-cancer compound or other specific binding components are known to those skilled in the art and may be selected depending on the type of assay protocol to be run. Incubation methods are also standard and are known to those skilled in the art.

In another embodiment, the term “contacting” means that the anti-cancer compound of the present invention is introduced into a subject receiving treatment, and the anti-cancer compound is allowed to come in contact with the cellular component in vivo.

As used herein, the term “treating” includes preventative as well as disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission. As used herein, the term “delaying” means stopping, hindering, slowing down, postponing, holding up or setting back. The term “treating” in the context of cancer includes the treatment of cancer metastases.

As used herein, the term “administering” refers to bringing a subject in contact with an anti-cancer compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

In one embodiment, the methods of the present invention comprise administering an anti-cancer compound as the sole active ingredient. However, also encompassed within the scope of the present invention are methods of cancer treatment comprising administering the anti-cancer compounds of the present invention in combination with other established cancer therapeutic drugs, including, but not limited to:
1) Alkylating agents—e.g. bischloroethylamines (nitrogen mustards), aziridines, alkyl alkone sulfonates, nitrosoureas, platinum compounds.
2) Antibiotic agents—e.g. anthracyclines, mitomycin C, bleomycin, dactinomycin, plicatomycin.
3) Antimetabolic agents—e.g. fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine.
4) Hormonal agents—e.g. synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.
5) Plant-derived agents—e.g. vinca alkaloids, podophyllotoxins, and taxanes.
6) Biologic agents—e.g. immuno-modulating proteins such as cytokines, monoclonal antibodies against tumor antigens, tumor suppressor genes, and cancer vaccines.

Thus, in one embodiment, the methods of the present invention comprise administering the anti-cancer compound of the present invention, in combination with an alkylating agent. In another embodiment, the methods of the present invention comprise administering the anti-cancer compound of the present invention, in combination with an antibiotic. In another embodiment, the methods of the present invention comprise administering the anti-cancer compound of the present invention, in combination with an antimetabolite. In another embodiment, the methods of the present invention comprise administering the anti-cancer compound of the present invention, in combination with a hormonal agent. In another embodiment, the methods of the present invention comprise administering the anti-cancer compound of the present invention, in combination with a plant-derived agent. In another embodiment the methods of the present invention comprise administering the anti-cancer compound of the present invention, in combination with a biologic agent.

Pharmaceutical Compositions

In one embodiment, the present invention provides a composition comprising the anti-cancer compound of the present invention and/or its analog, derivative, isomer, metabolite, pharmaceutically acceptable salt, pharmaceutical product, hydrate, N-oxide, impurity, prodrug, polymorph, crystal, or any combination thereof.

In another embodiment, the present invention provides a pharmaceutical composition comprising the anti-cancer compound of the present invention and/or its analog, derivative, isomer, metabolite, pharmaceutical product, hydrate, N-oxide, impurity, prodrug, polymorph, crystal, or any combination thereof; and a suitable carrier or diluent.

As used herein, “pharmaceutical composition” means therapeutically effective amounts of the anti-cancer together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or Lyophilized or otherwise dried formulations and include diluents of various buffer content (e.g., Tris-HCI., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Controlled or sustained release compositions include formulation in lipophilic depots (e.g., fatty acids, waxes, oils).

Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, intravaginally, intracranially and intratumorally.

Further, as used herein “pharmaceutically acceptable carriers” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Other embodiments of the compositions of the invention incorporate particulate forms, protective coatings, protease inhibitors or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

The pharmaceutical preparation can comprise the anti-cancer agent alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories.

Pharmaceutically acceptable carriers include gums, starches, sugars, cellulosic materials, and mixtures thereof. The pharmaceutical preparation containing the anti-cancer agent can be administered to a subject by, for example, subcutaneous implantation of a pellet; in a further embodiment, the pellet provides for controlled release of anti-cancer agent over a period of time. The preparation can also be administered by intravenous, intraarterial, or intramuscular injection of a liquid preparation, oral administration of a liquid or solid preparation, or by topical application. Administration can also be accomplished by use of a rectal suppository or a urethral suppository.

The pharmaceutical preparations of the invention can be prepared by known dissolving, mixing, granulating, or tablet-forming processes. For oral administration, the anti-cancer agents or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration. (subcutaneous, intravenous, intraarterial, or intramuscular injection), the anti-cancer agents or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Typically, such compositions are prepared as aerosols of the polypeptide delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the anti-cancer agents or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid).

For use in medicine, the salts of the anti-cancer will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

The following examples are presented in order to more fully illustrate the preferred embodiments of the invention. They should in no way be construed, however, as limiting the broad scope of the invention.

### EXPERIMENTAL DETAILS SECTION

### EXAMPLE 1

### Experimental Methods

Cell Lines

The origins of the cell lines used in the studies described herein are shown in Table 1 below:

TABLE 1

| Cell line | Morphology | Origin | Patient |
|---|---|---|---|
| LNCaP | Epithelial | Needle | 50-year-old white male with |
| | | aspiration | tage D1 prostatic cancer |
| | | biopsy of left | |
| | | supraclavicular | |
| | | lymph node | |
| DU 145 | Epithelial | Metastatic CNS | 69-year-old white male with |
| | | lesion | metastatic carcinoma of the |
| | | | prostate and a 3 year history of |
| | | | lymphocytic leukemia |
| PC-3 | Epithelial | Prostatic | 62-year-old male Caucasian with |
| | | metastatic bone | grade IV prostatic |
| | | marrow | adenocarcinoma |
| PPC-1 | Epithelial | Transurethral | 67-year-old black male with |
| (primary prostate | | resection of the | stage D2 poorly differentiated |
| carcinoma-1) | | prostate | adenocarcinoma of prostate |
| TSU | Epithelial | Metastatic | 73-year-old male Japanese with a |
| | | tumor in a | moderately differentiated prostatic |
| | | cervical lymph node | adenocarcinoma |

Cell Culture

Prostate cancer cell lines were obtained from ATCC. All cells were grown in RPMI-1640 medium containing 2 mM L-glutamine supplemented with 10% fetal bovine serum (FBS) and maintained in a 5% CO2/95% air humidified atmosphere at 37° C.

Assay for Cell Growth Inhibition (Sulforhodamine B Assay)

Cells were plated on 96-well plates and incubated with drug-containing culture medium (200 μL/well) for 4 (DU 145, PC-3, PPC-1, and TSU) or 6 (LNCaP) days. Medium was replaced with freshly prepared batches every other day during the incubation. At the end of drug treatment, an aliquot of 50 μL of cold (4° C.) trichloroacetic acid (TCA, 50%) was gently layered on the top of growth medium in each well to make a final TCA concentration of 10%. The mixtures were incubated at 4° C. for 1 hour, and then washed 5 times with tap water to remove TCA, growth medium, low-molecular-weight metabolites, and serum proteins. The plates were air dried overnight. Next, fixed cells were stained with 50 μL of SRB solution (0.4%, wt/vol) for 10 minutes. After staining, SRB solution was decanted, and plates were quickly rinsed 5 times with 1% acetic acid to remove unbound dye and air dried overnight. The cellular protein-bound SRB was then dissolved with 200 μL unbuffered Tris base (10 mM, pH 10.5) for 30 minutes on a rocking platform shaker, and absorbance at 540 nm was measured by a plate reader.

Percentage of cell survival was calculated by absorbance at 540 nm in testing wells divided by absorbance in negative control wells (medium without the test compound). Percentages of cell survival versus drug concentrations were plotted and the concentration of drug that inhibited cell growth by 50% (IC50) was determined by nonlinear regression using WinNonlin (Pharsight Corporation, Mountain View, Calif.).

### EXAMPLE 2

### Effect of Haloacetamide Substituted Compounds in Different Cell Lines

METHODS: LNCaP, DU145, PC-3, TSU, and PPC-1 cells were cultured in 96-well plates and treated with increasing concentrations of the compound of interest for 4 days. Cell survival was determined by the sulforhodamine B assay and was plotted as a percentage of control (drug-free wells) versus drug concentration. The concentration of drug that inhibited cell growth by 50% (IC50) was determined by nonlinear regression. Known anticancer drugs were used as cytotoxic positive controls.

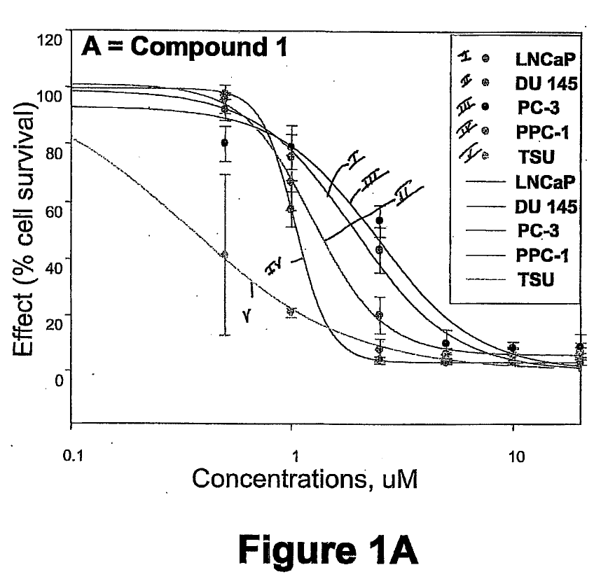

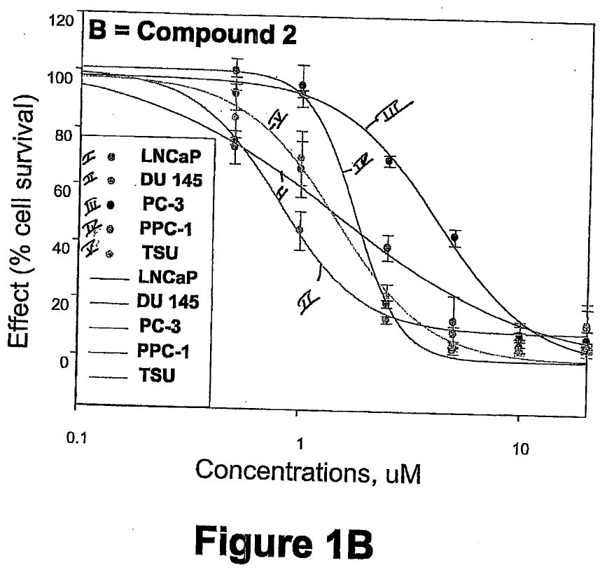

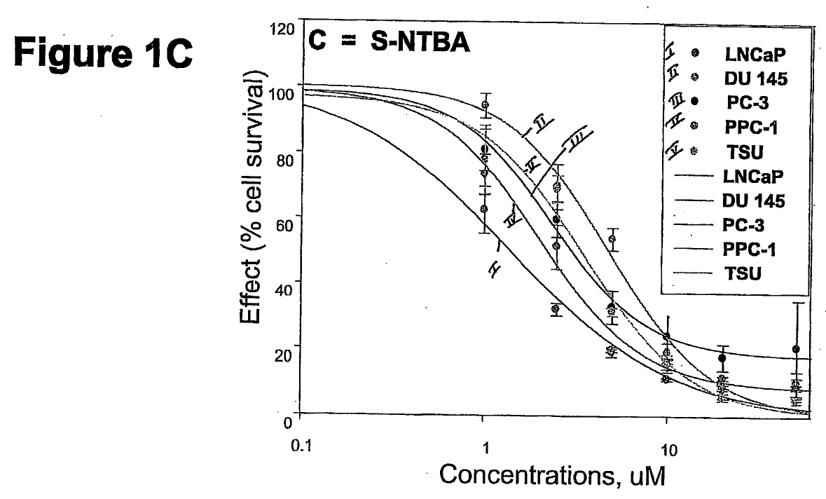

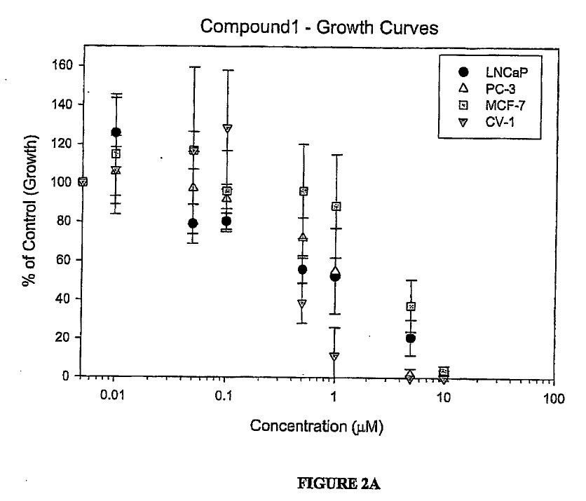

RESULTS: The IC₅₀s of Compounds A and B, as well as S-NTBA, 5-FU and Melphalan in prostate cancer cell lines DU 145, PC-3, TSU, PPC-1 and LNCaP are shown in Table 1. The cytotoxicity of compounds A, B and S-NTBA in different cell lines are shown in FIG. 1A-C, respectively. Compounds A and B demonstrated IC₅₀ values in the low micro-molar range in inhibiting the growth of all of five prostate cancer cell lines.

LNCaP cells were not more sensitive to compounds A and B than other cell lines. The IC₅₀s from one-day treatment and 4 or 6 days treatment did not show significant difference, indicating that the growth inhibitory activity of these compounds was not likely a reversible process.

These studies indicated that compounds A and B may have potential as chemotherapeutic agents for the treatment of prostate cancer.

TABLE 1
Prostate Cancer Cell Lines

| | | Prostate Cancer Cell Lines | | | | |
|---|---|---|---|---|---|---|
| Name | Structure | DU145 | PC-3 | TSU | PPC-1 | LNCaP |
| Compound A (μM) (Compound 15 Scheme 1) | 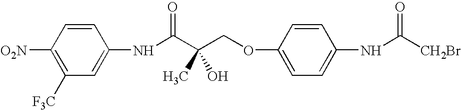 | 1.3±0.3 | 2.41±0.6 | 0.4±0.3 | 1.1±0.1 | 1.1±0.2 |
| Compound B (μM) (Compound 14 Scheme 1) | 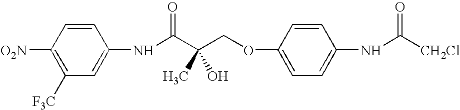 | 0.9±0.1 | 4.2±0.2 | 1.4±0.4 | 1.8±0.1 | 4.4±0.8 |
| S-NTBA (μM) | 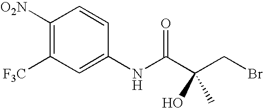 | 4.7±0.3 | 3.1±0.5 | 3.5±0.2 | 2.2±0.2 | 1.3±0.2 |
| 5-FU (μM) | | 2.6±0.9 | 12.1±0.9 | 2.9±0.9 | 5.5±0.3 | 0.9±0.3 |
| Melphalan (μM) | | 31.0±4.8 | 30.4±3.1 | 4.0±0.2 | 16.2±1.8 | 10.3±0.1 |

### EXAMPLE 3

### Effect of Haloacetamide Substituted Compounds on Cell Growth

Growth Curve:

MATERIALS: DMSO is the vehicle control and the solvent for Compound A and Compound B.

METHODS: Cells were plated at 5-10×10⁴ cells/well in five 6-well plates and incubated at 37° C., 5% CO₂ for 24 h to allow the cells sufficient time to attach and be in log phase growth at the start of the experiment. The media was aspirated from four of the plates and replaced with media containing vehicle control (DMSO) or drug dissolved in DMSO. The total volume of DMSO/drug added to each well was equal to 0.1% of the media volume in each well. LNCaP, PC-3, MCF-7, and CV-1 cells were treated with vehicle control, and increasing concentrations of Compound A and Compound B (0.01, 0.05, 0.1, 0.5, 1.0, 5.0, and 10.0 μM). Three wells were treated with the same concentration of the drugs or DMSO for each treatment condition listed above. The cells from the remaining 6-well plate were collected and counted to determine plating efficiency. The 6-well plates containing DMSO/drug were incubated for 120 h at 37° C., 5% CO₂. After 120 h, the media from each well was collected along with trypsinized cells and centrifuged at 150×g for 4 min. The cells were resuspended in 1 mL of media, from which 90 μl was taken and combined with 10 μl trypan blue for counting on a hemacytometer.

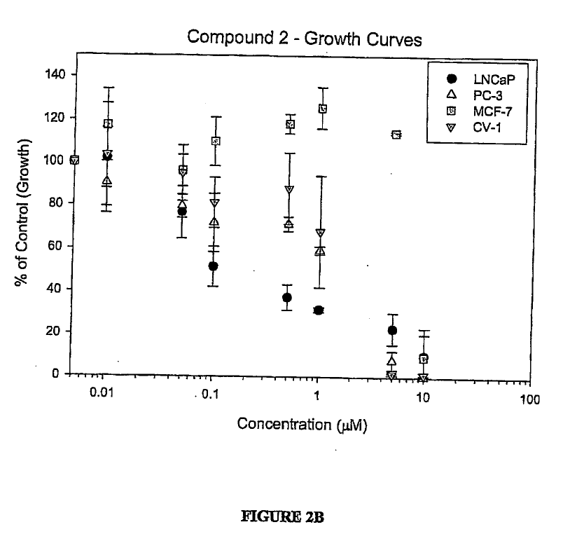

RESULTS: The results are presented in FIG. 2. Results indicate that the haloacetamides are potent cytotoxic agents. Compound A exhibits non-selective growth inhibitory activity against various cancer cell lines in vitro where LNCaP (AR-dependent) cells are inhibited by approximately the same molar concentration of Compound A as the PC-3, MCF-7 and CV-1 cells (which are prostate, breast, and monkey kidney cell lines, respectively, none of which are dependent on the AR for growth) (FIG. 2A). Compound B appears to exhibit some selectivity in that LNCaP cells are approximately 10-fold more sensitive than the PC-3 or CV-1 cells. Only at very high concentrations (i.e. >5 micromolar) are the MCF-7 cells sensitive to Compound B (FIG. 2B).

Tunnel Assay:

MATERIALS: In Situ Cell Death Detection Kit, Fluorescein (Roche).

METHODS: DNA fragmentation of apoptotic cells was monitored by the TUNEL assay as described by the supplier. Briefly, LNCaP cells were plated at 2×10⁵ cells/well in 2-well chamber slides and incubated at 37° C., 5% CO₂ for 24 h to allow the cells sufficient time to attach and be in log phase growth at the start of the experiment. The media was aspirated and replaced with media containing vehicle control (DMSO) or drug dissolved in DMSO. The total volume of DMSO/drug added to each well was equal to 0.1% of the media volume in each well. LNCaP cells were treated with vehicle control, and increasing concentrations of Compound A and Compound B (0.1, 1.0, and 10.0 μl) for 24-48 h. Two wells were treated with the same concentration of the drugs or DMSO for each treatment condition listed above. The media was collected along with the trypsinized cells and centrifuged at 150 ×g for 4 min. The cells were resuspended in 50 μl PBS, pipetted onto poly-lysine coated slides, and then fixed in 4% methanol-free formaldehyde in PBS (H 7.4) for 25 min at 4° C. Cells were permeabilized in 0.2% Triton X-100 in PBS for 5 min at room temperature. Terminal deoxynucleotidyl transferase labeling of 3′-ends of DNA strand breaks was performed using fluorescein-12-dUTP with an apoptosis detection system. Following end labeling, cells were then washed with PBS containing 0.1% Triton X-100 and 5 mg/ml albumin from bovine serum (BSA). All cells were stained with 1 μg/ml propidium iodide for 15 min. Green and red fluorescence emissions were observed microscopically using 520 nm and >620 nm filters, respectively.

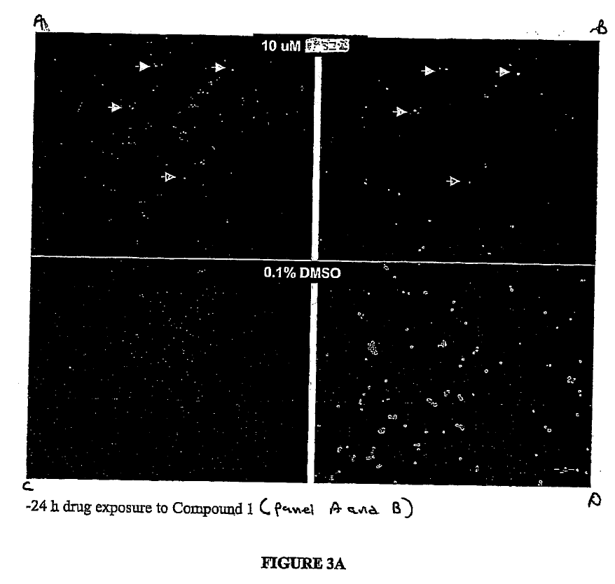

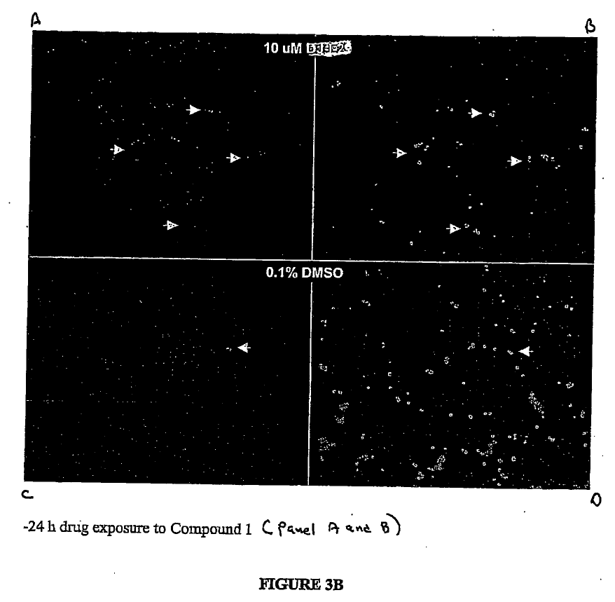

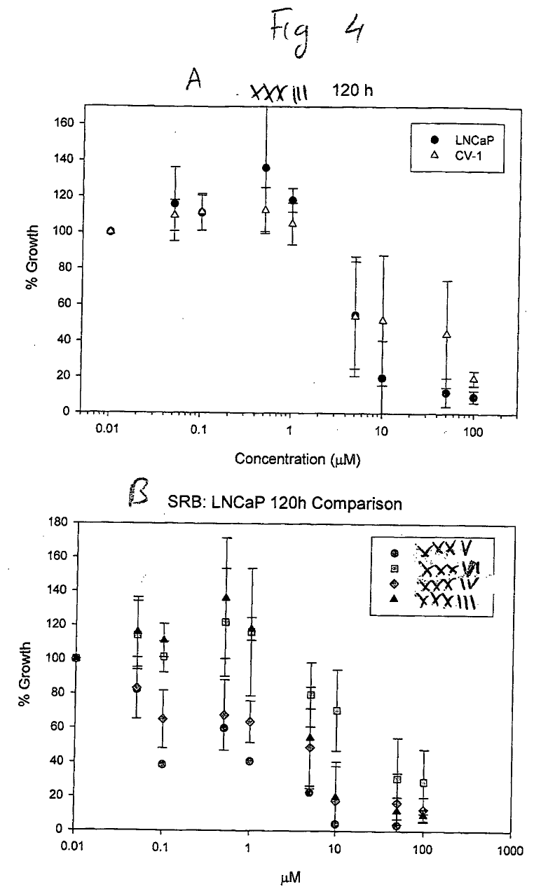

RESULTS: The TUNEL assay is used to determine whether cells are undergoing apoptosis (cell death mechanism) as a result of drug treatment. During apoptosis the DNA of affected cells is fragmented, leaving 3′ and 5′ ends exposed. TUNEL assay incorporates a dye that labels the 3′ ends of such DNA fragments which are then visualized by fluorescence. Results show that cells exposed to Compound A for 24 hours exhibit green fluorescence (relative to the 0.1% DMSO vehicle control cells) (FIGS. 3A and B). The green fluorescene demonstrates that the cells have fragmented DNA and are undergoing apoptosis. There are also fewer cells stained with propidium iodide (relative to vehicle control) which is a further indication that many of the cells have died and floated away. Results for Compound B were similar (data not shown).

Without wishing to be bound to any particular mechanism or theory, one possible mechanism of action for haloacetamide compounds such as compounds A and B is that they alkylate cellular nucleophiles, the brominated derivative (Compound A) being more potent (more reactive) than the chlorinated derivative (Compound B), thus requiring a higher concentration of Compound B before apoptosis is initiated.

### EXAMPLE 4

### Inhibition of Cell Growth by Compound XXXIII-XXXVII of the Invention

The experiment was conducted using the following compounds:

| Compound No. | Structure | Ki (nM) |
|---|---|---|
| XXXIII | 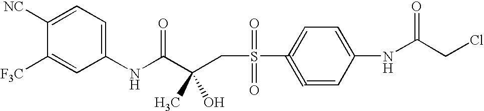 | 14 |
| XXXIV | 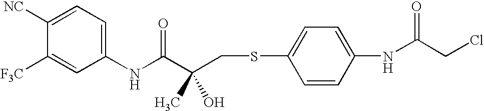 | 6.54 |
| XXXV | 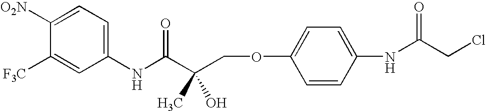 | 1.9 |
| XXXVI | 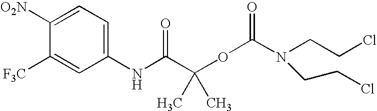 | DNB |

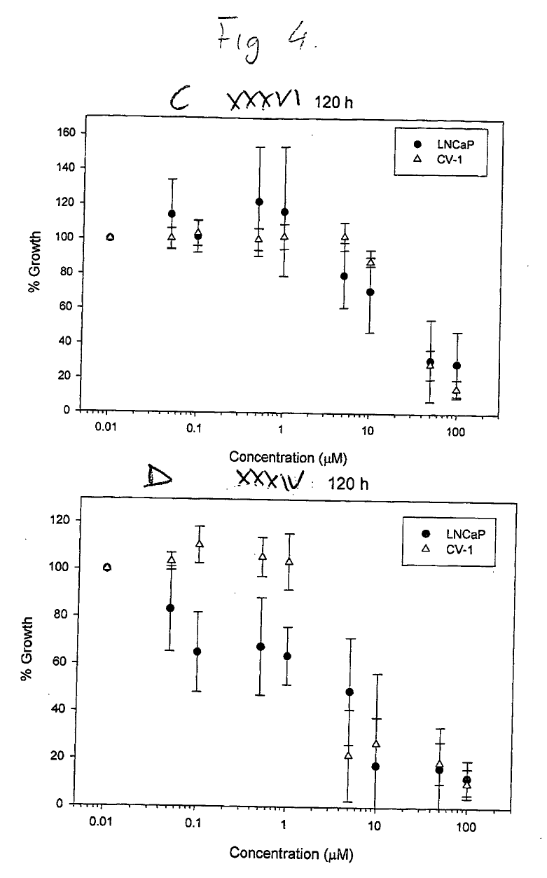

LNCap and CV-1 cells were treated by the above compounds for 120 h. FIGS. 4A-D show dose dependent inhibition of the cells growth by all compounds of the invention.

### EXAMPLE 5

### Synthesis of the Compounds of the Invention

Compounds 10-24 of the present invention were synthesized according to the reactions set forth in Scheme 1 below: 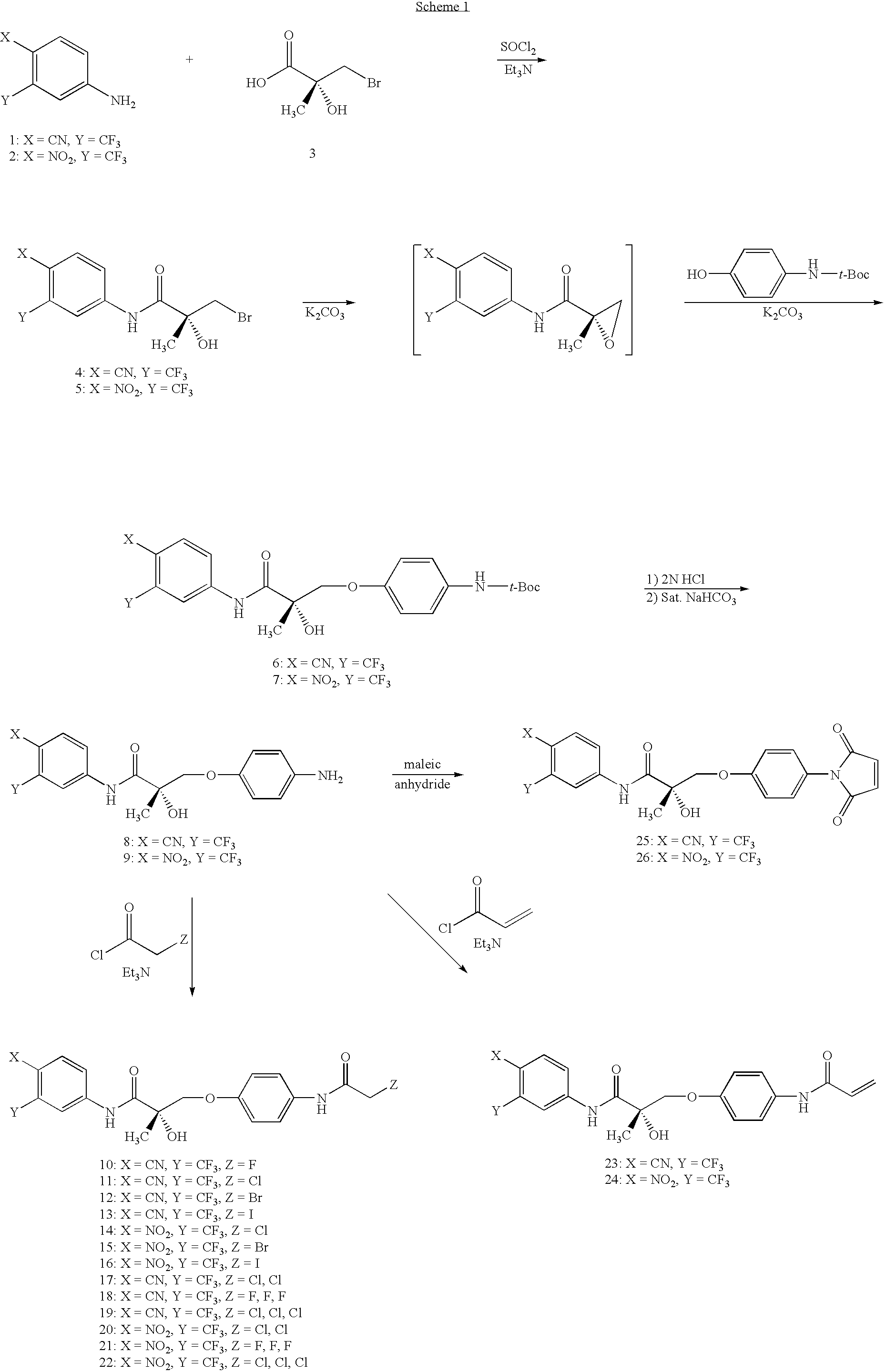
General Procedure for the Synthesis of Bromoanilide Compounds (4, 5, and 28)

To a cold solution of bromoacid^{I} 3 (0.29 mol) in 300 mL of THF was added SOCl₂ (0.39 mol) in a dropwise manner under an argon atmosphere. The reaction mixture was stirred for 3 h under an ice-water bath and then Et₃N (0.39 mol), aniline (1, 2, or 25², 0.19 mol) were added. The reaction mixture was stirred for 20 h at room temperature and concentrated under reduced pressure to give a solid which was treated with 300 mL of H₂O. The solution was extracted with EtOAc (2×400 mL) and combined EtOAc extracts were washed with saturated NaHCO₃ solution (2×300 mL) and brine (300 mL), successively. The organic layer was dried over MgSO₄ and concentrated under reduced pressure to give an oil which was purified by column chromatography using CH₂Cl₂/EtOAc (8:2) to give a solid which was recrystallized from EtOAc/hexane to give a target compound.

1) Kirkovsky, L.; Mukherjee, A.; Yin, D.; Dalton, J. T.; Miller, D. D. Chiral nonsteroidal affinity ligands for the androgen receptor. 1. Bicalutamide analogues bearing electrophilic groups in the B aromatic ring. J. Med. Chem 2000, 43(4), 581-590.

2) Van Dort, M. E.; Robins, D. M.; Wayburn, B. Design, Synthesis, and Pharmacological Characterization of 4-[4,4-Dimethyl-3-94-hydroxybutyl]-5-oxo-2-thioxo-1-imidazolidinyl]-2-iodobenzonitrile as a High-Affinity Nonsteroidal Androgen Receptor Ligand. 2000, 43, 3344-3347.

General Procedure for the Synthesis of t-Boc-Aminophenoxy Compounds (6 and 7)

To a solution of bromoanilide (20.21 mmol) in 200 mL of acetone was added anhydrous K₂CO₃ (60.6 mmol). The reaction mixture was heated to reflux for 2 h and concentrated under reduced pressure to give a solid. The resulting residue was treated with K₂CO₃ (40.42 mmol), 4-t-Boc-aminophenol (20.21 mmol), 200 mL of methyl ethyl ketone. The reaction mixture was heated to reflux for 3.5 h and concentrated under reduced pressure to give a solid. Solid was treated with H₂O (150 mL) and extracted with CH₂Cl₂ (2×80 mL). The combined CH₂Cl₂ extracts were washed with 10% NaOH (2×100 mL), H₂O (100 mL), successively. The organic layer was dried over MgSO₄ and concentrated under reduced pressure to give a solid which was purified by column chromatography using EtOAc/hexane (1:2) to give a target compound.

General Procedure for the Synthesis of Aniline Compounds (8 and 9)

To a solution of N-t-Boc protected compound (8.01 mmol) in 30 mL of CH₃OH was added 30 mL of 2N HCl solution in diethyl ether. The reaction mixture was stirred overnight at room temperature and concentrated under reduced pressure to give an oil. Oil was treated with 30 mL of saturated NaHCO₃ solution and extracted with EtOAc (2×30 mL). The combined extracts were washed with 30 mL of brine, dried over MgSO₄, and concentrated under reduced pressure to give a target compound.

General Procedure for the Synthesis of Alpha-Halogeno and Vinyl Ketone Compounds (11-24)

To a cold solution of haloacetyl chloride or acryloyl chloride (6.88 mmol) in 50 mL of CH₂Cl₂ was added Et₃N (9.18 mmol) and aniline compound (4.59 mmol). The reaction mixture was stirred overnight at room temperature, washed with H₂O (2×30 mL), and dried over MgSO₄. The solvent was removed under reduced pressure to give an oil which was purified by column chromatography using EtOAc/hexane (8:2) to give an oil. Oil was crystallized from EtOAc/hexane to give a target compound.

Compound 15 (bromoacetamido derivative) corresponds to Compound A in Examples 2 and 3 above. Compound 14 (chloroacetamido derivative) corresponds to Compound B in Examples 2 and 3 above.

General Procedure for the Synthesis of Maleamide Compounds (25 and 26)

A solution of aniline compound (0.46 mmol) in 50 mL of CH₂Cl₂ and 0.5 mL of DMF was heated to reflux overnight. After cooling, the solution was concentrated under reduced pressure to give an oil which was treated with 50 mL of CH₂Cl₂, and washed with H₂O (2×30 mL). The organic layer was dried over MgSO₄ and concentrated under reduced pressure to give an oil which was purified by column chromatography using EtOAc/hexane (3:2) to give a target compound.

The Synthesis of α-Fluoroacetamide Compound (10)

Compound 10 was synthesized according to the reaction set forth in Scheme 2 below:

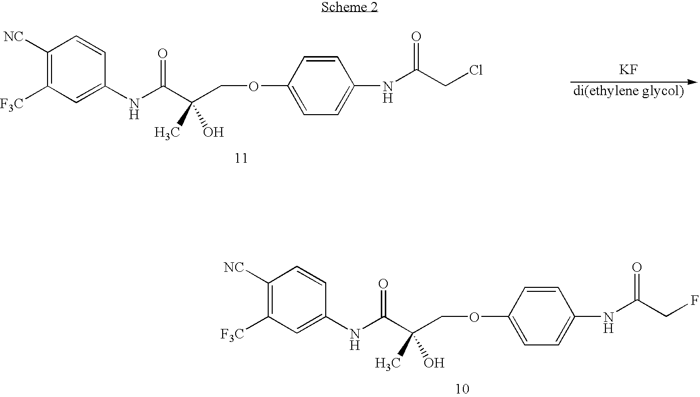

A stirred mixture of α-chloroamide 11 (130 mg, 0.32 mmol), potassium fluoride (46 mg, 0.80 mmol), and 5 mL of di(ethylene glycol) was heated to 125-135° C. for 2 h in the sealed tube. The reaction mixture was diluted with 20 mL of H₂O, extracted with CH₂Cl₂ (2×20 mL). The combined CH₂Cl₂ extracts were dried over MgSO₄ and concentrated under reduced pressure to give an oil which was purified by flash column chromatography using EtOAc/hexane (1:1) to give 73 mg (53.0%) of 10 as a yellowish solid.

Synthesis of Benzenesulfonyl Fluoride Compound (36)

Compound 36 was synthesized according to the reaction set forth in Scheme 3 below: 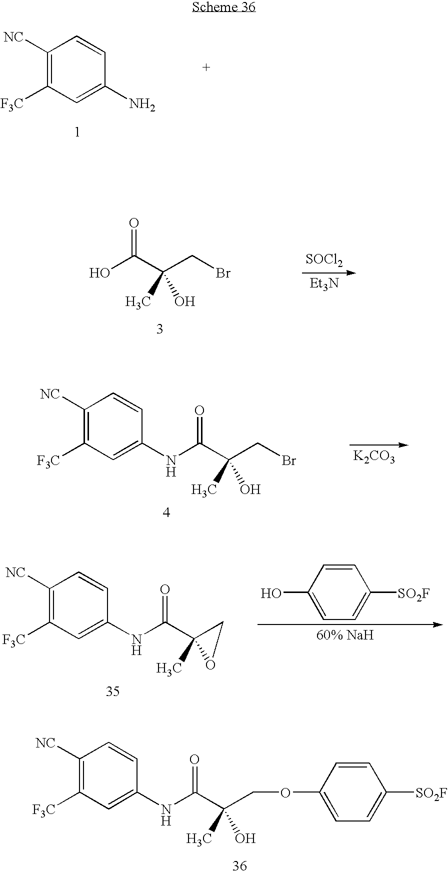
4-[((2S)-3-{[4-cyano-3-(trifluoromethyl)phenyl]amino}-2-hydroxy-2-methyl-3-oxopropyl)oxy]benzenesulfonyl fluoride (36)

To a solution of bromoamide (4, 1.0 g, 2.85 mmol) in 40 mL of acetone was added anhydrous K₂CO₃ (1.18 g, 8.54 mmol). The reaction mixture was heated to reflux for 1 h and concentrated under reduced pressure to give a solid. The solid was treated with 40 mL of H₂O and extracted with EtOAc (2×30 mL). The combined EtOAc extracts were washed with brine (1×30 mL), dried over MgSO₄, and concentrated under reduced pressure to give an epoxide compound 35 as an oil. Without further purification, a solution of epoxide in 10 mL of THF was added to a suspension of the sodium salt of 4-fluorosulfonyl phenol [prepared from a 60% NaH dispension (0.11 g, 3.13 mmol) in oil and 4-fluorosulfonyl phenol^{I} (0.5 g, 2.85 mmol) in 10 mL of THF] and stirred at room temperature overnight. The reaction mixture was concentrated under reduced pressure, treated with H₂O (10 mL), and extracted with EtOAc (2×20 mL). The combined EtOAc extracts were washed with 10% NaOH (2×20 mL), brine (20 mL), and dried over MgSO₄. The solvent was removed under reduced pressure to give an oil which was purified by column chromatography using EtOAc/hexane (1:1) to give a target compound (36, 0.25 g, 19.7%) as a colorless oil: ¹H NMR (CDCl₃/TMS) □ 1.66 (s, 3H, CH₃), 3.41 (s, 1H, OH), 4.15 (d, J=9.2 Hz, 1H, CH), 4.58 (d, J=9.2 Hz, 1H, CH), 7.11 (d, J=8.9 Hz, 2H, ArH), 7.82 (d, J=8.5 Hz, 1H, ArH), 7.94-8.13 (m, 3H, ArH), 8.14 (s, 1H, NH); MS (ESI): m/z 445.1 [M−H]⁻; Anal. Calcd. for C₁₈H₁₄F₄N₂O₅S. 0.25 EtOAc: C 48.72, H 3.44, N, 5.98. Found: C 48.93, H 3.52, N 5.83.

1) Steinkopf, W. Aromatische sulfofluoride. J. Prakt. Chem. 1927, 117, 21.

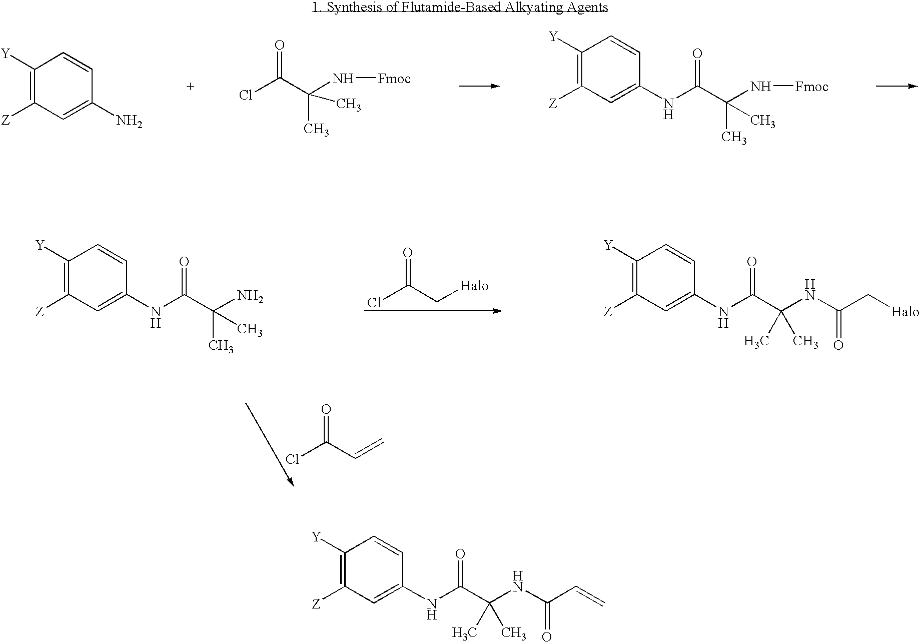

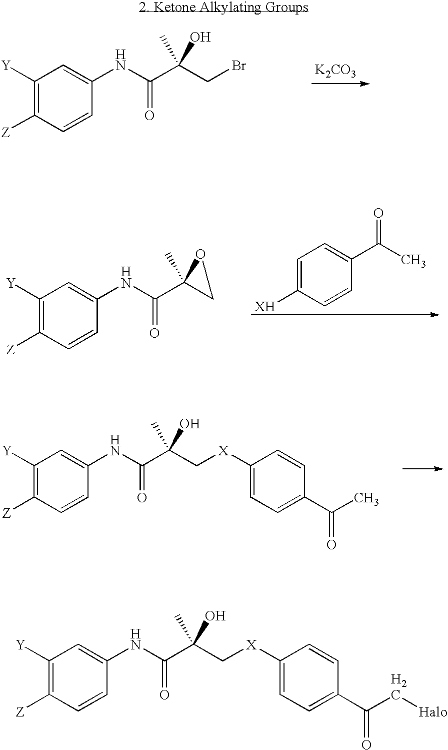

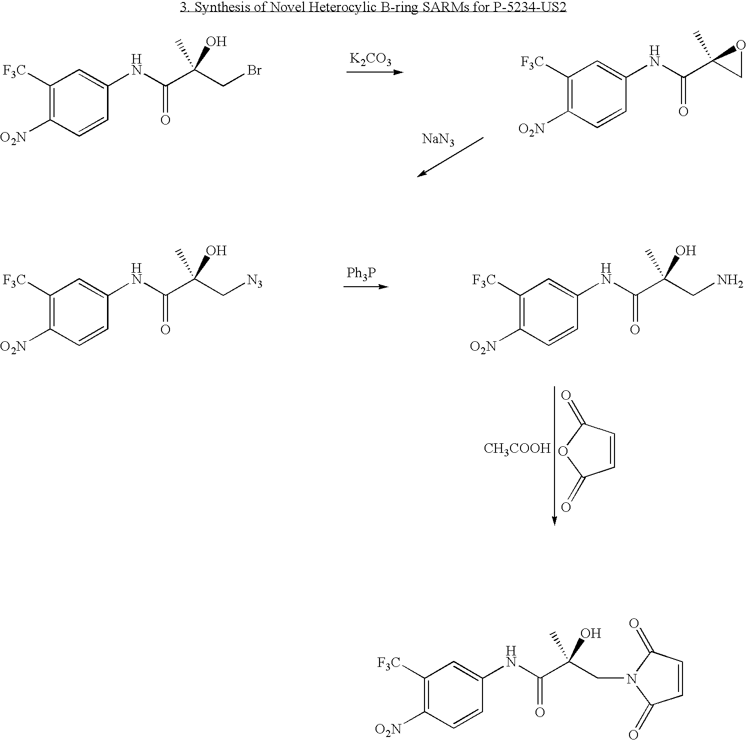

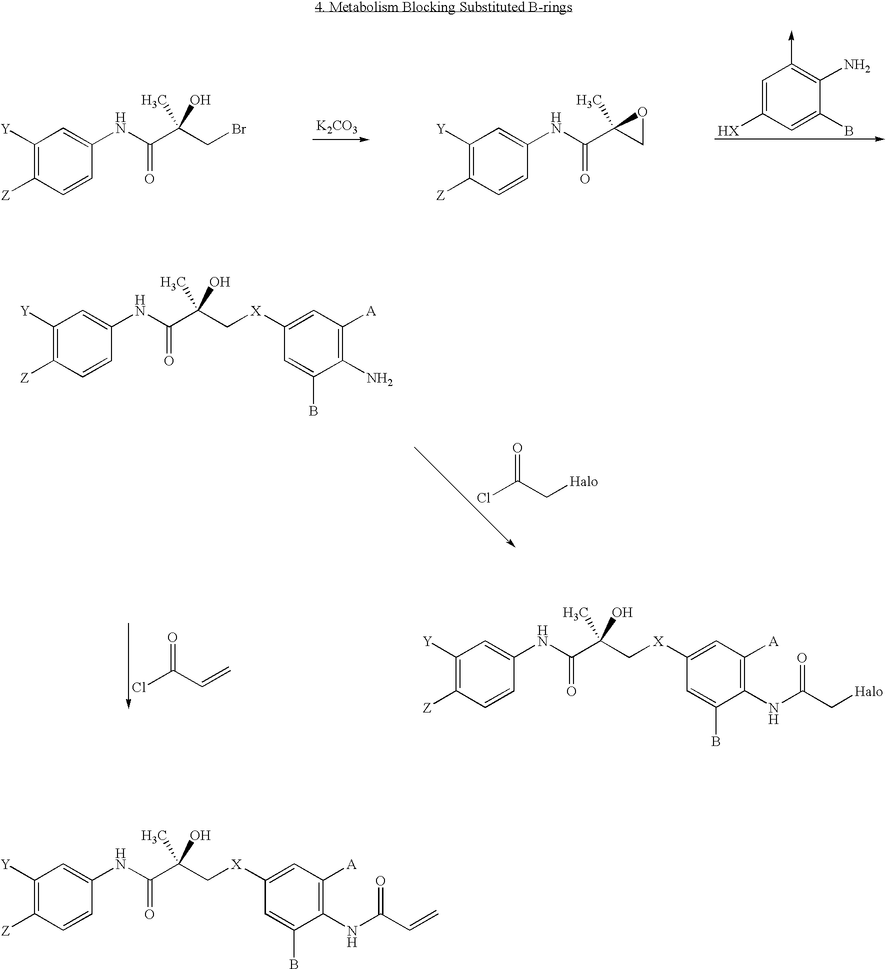

A and B are each independently H, CH₃ or halogen and X is O, S or SO₂.

The physical properties of several of the compounds of the present invention are summarized in Table 2 below:

| COMD | | | MASS | | | YIELD |
|---|---|---|---|---|---|---|
| NO | STRUCTURES | ¹H NMR | [M−H]⁻ | C, H, N | MP (° C.) | (%) |
| 4 | 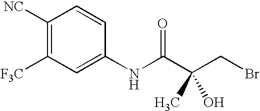 | (CDCl₃) δ 9.04(s, 1H, NH), 8.12(d, J=2.1 Hz, 1H, ArH), 7.99(dd, J=8.4, 2.1 Hz, 1H, ArH), 7.85 (d, J=8.4 Hz, 1H, ArH), 4.05 (d, J=10.8 Hz, 1H, CH), 3.63 (d, J=10.8 Hz, 1H, CH), 3.11 (s, 1H, OH), 1.66 (s, 3H, CH₃) | 450.0 | C₁₂H₁₀BrF₃N₂O₂: C, 41.05; H, 2.87; N, 7.98 Found: C, 41.25; H, 2.89; N, 8.01. | 124-126 | 77.0 |
| 5 | 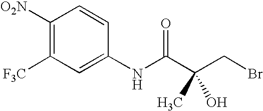 | (DMSO-d₆) δ 10.54 (s, 1H, NH), 8.54 (d, J=2.1 Hz, 1H, ArR), 8.34 (dd, J=9.0, 2.1 Hz, 1H, ArH), 8.18(d, J=9.0 Hz, 1H, ArH), 6.37 (s, 1H, OH), 3.82 (d, J=10.4 Hz, 1H, CH), 3.58 (d, J=10.4 Hz, 1H, CH), 1.48 (s, 3H, CH₃) | 370.8 [M]⁺ | C₁₁H₁₀BrF₃N₂O₄: C, 35.60; H, 2.72; N, 7.55 Found: C, 35.68; H, 2.72; N, 7.49. | 98-100 | 80.0 |
| 28 | 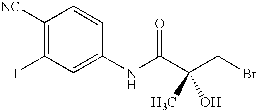 | (CDCl₃) δ 8.81(s, 1H, NH), 8.27(d, J=2.0 Hz, 1H, ArH), 7.70(dd, J=8.5, 2.0 Hz, 1H, ArH), 7.56 (d, J=8.5 Hz, 1H, ArH), 4.01 (d, J=10.5 Hz, 1H, CH), 3.59 (d, J=10.5 Hz, 1H, CH), 3.01 (s, 1H, OH), 1.62 (s, 3H, CH₃) | 409.3 [M+H]⁺ | C₁₁H₁₀BrIN₂O₂: C, 32.30; H, 2.46; N, 6.85 Found: C, 32.42; H, 2.43; N, 6.75. | 157-160 | 57.2 |
| 6 | 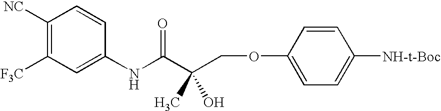 | (CDCl₃) δ 9.17 (s, 1H, NH), 8.10 (s, 1H, ArH), 7.96 (d, J=8.4 Hz, 1H, ArH), 7.80 (d, J=8.4, Hz, 1H, ArH), 7.27 (d, J=8.1 Hz, 1H, ArH), 6.84 (d, J=8.1 Hz, 2H, ArH), 6.43 (bs, 1H, NH), 4.42 (d, J=9.0 Hz, 1H, CH), 3.95 (d, J=9.0 Hz, 1H, CH), 3.54 (s, 1H, OH), 1.57 (s, 3H, CH₃), 1.50 (s, 9H, CH₃) | 478.1 | C₂₃H₂₄F₃N₃O₅, H₂O: C, 55.53; H, 5.27; N, 8.45 Found: C, 55.21; H, | | |
| # 4.94: N, 816. | 156-158 | 57 | | | | |
| 7 | 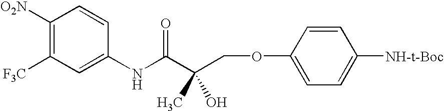 | (CDCl₃) δ 9.20 (s, 1H, NH), 8.10 (s, 1H, ArH), 8.02-8.01 (m, 2H, ArH), 7.27 (d, J=8.6 Hz, 2H, ArH), 6.84 (d, J=8.6, Hz, 2H, ArH), 4.42 (d, J=9.0 Hz, 1H, CH), 3.95 (d, J=9.0 Hz, 1H, CH), 3.54 (s, 1H, OH), 1.58 (s, 3H, CH₃), 1.51 (s, 9H, CH₃) | 497.8 | C₂₂H₂₄F₃N₃O₇: C, 52.91; H, 4.84; N, 8.41 Found: C, 52.77; H, 4.91; N, 8.42. | 145-147 | 58 |
| 8 | 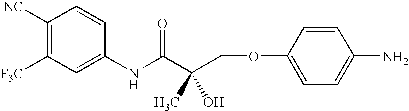 | (DMSO-d₆) δ 10.54 (bs, 1H, NH), 8.57 (d, J=1.8 Hz 1H, ArH), 8.32 (dd, J=8.7, 1.8 Hz, 1H, ArH), 8.11 (d, J=8.7 Hz, 1H, ArH), 6.63 (d, J=9.0 Hz, 2H, ArH), 6.48 (d, J=9.0 Hz, 2H, ArH), 6.17 (bs, 1H, OH), 4.61 (bs, 1H, NH₂), 4.08 (d, J=9.6 Hz, 1H, CH₂), 3.85 (d, J=9.6 Hz, 1H, CH₂), 1.18 (s, 3H, CH₃) | 398.0 | C₁₇H₁₆F₃N₃O₅, 0.5C₄H₈O₂: C, 51.47; H, | | |
| # 4.36; N, 9.48 Found: C, 51.58; H, 4.36; N, 9.91. | 177-179 | 91.1 | | | | |
| 9 | 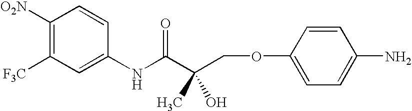 | (CDCl₃) δ 8.38 (d, J=1.8 Hz, 1H, ArH), 8.14 (dd, J=8.7, 1.8 Hz, 1H, ArH), 8.04 (d, J=8.7 Hz, 1H, ArH), 7.11 (d, J=9.0 Hz, 2H, ArH), 6.98 (d, J=9.0 Hz, 2H, ArH), 4.32 (d, J=9.6 Hz, 1H, CH), 4.03 (d, J=9.6 Hz, 1H, CH), 1.52 (s, 3H, CH₃). | 398.0 | C₁₇H₁₆F₃N₃O₅, 0.5C₄H₈O₂: C, 51.47; H, 4.36; N, 9.48 Found: C, 51.58; H, 4.36; N, 9.91. | 138-140 | 92.3 |
| 10 | 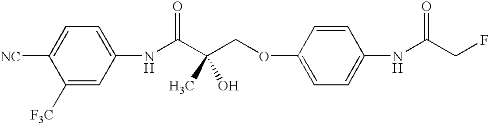 | (CDCl₃) δ 9.25 (bs, 1H, NH), 8.12 (d, J=1.5 Hz, 1H, ArH), 7.97 (dd, J=8.4, 1.5 Hz, 1H, ArH), 7.79 (d, J=8.4 Hz, 1H, ArH), 7.46 (d, J=9.0 Hz, 2H, ArH), 6.87 (d, J=9.0 Hz, 2H, ArH), 4.99 (s, 1H, CH₂F), 4.83 (s, 1H, CH₂F), 4.43 (d, J=9.0 Hz, CH₂), 3.97 (d, J=9.0 Hz, CH₂), 3.81 (bs, 1H, OH), 1.57 (s, 3H, CH₃) | 438.1 | C₂₀H₁₇F₄N₃O₄, 0.3C₄H₈O₂: | | |
| # C, 54.67; H, 4.20; N, 9.02 Found: C, 54.82; H, 4.28; N, 8.99. | 67-69 | 53 | | | | |
| 11 | 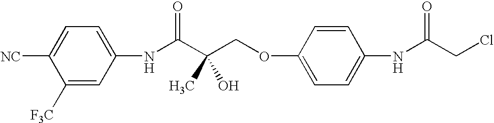 | (CDCl₃) δ 9.18 (bs, 1H, NH), 8.19 (bs, 1H, NH), 8.11 (d, J=1.5 Hz, 1H, ArH), 7.96 (dd, J=8.7, 2.1 Hz, 1H, ArH), 7.80 (d, J=8.7 Hz, 2H, ArH), 7.44 (d, J=9.0 Hz, 2H, ArH), 6.88 (d, J=9.0 Hz, 2H, ArH), 4.45 (d, J=9.3 Hz, CH₂), 4.18 (s, 2H, CH₂Cl), 3.98 (d, J=9.3 Hz, CH₂), 3.60 (bs, 1H, OH), 1.59 (s, 3H, CH₃) | 454.1 | C₂₀H₁₇ClF₃N₃O₄, 0.25H₂O: C, 52.18; | | |
| # H, 3.83; N, 9.13 Found: C, 52.08; H, 3.84; N, 8.93. | 68-70 | 95 | | | | |
| 12 | 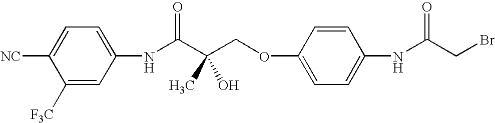 | (CDCl₃) δ 9.18 (bs, 1H, NH), 8.19 (bs, 1H, NH), 8.11 (d, J=2.1 Hz, 1H, ArH), 7.96 (dd, J=8.7, 2.1 Hz, 1H, ArH), 7.80 (d, J=8.7 Hz, 2H, ArH), 7.44 (d, J=9.0 Hz, 2H, ArH), 6.88 (d, J=9.0 Hz, 2H, ArH), 4.45 (d, J=9.3 Hz, CH₂), 4.18 (s, 2H, CH₂Cl), 3.98 (d, J=9.3 Hz, CH₂), 3.60 (bs, 1H, OH), 1.59 (s, 3H, CH₃) | 498.1 | C₂₀H₁₇BrF₃N₃O₄, 0.3H₂O: C, 47.50; H, 3.51; | | |
| # N, 8.31 Found: C, 47.36; H, 3.34; N, 8.32. | 77-79 | 75 | | | | |
| 13 | 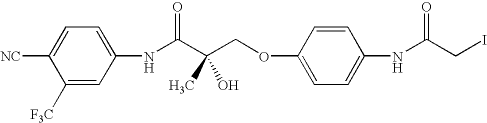 | (CD₃OD) δ 8.37 (d, J=1.5 Nz, 1H, ArH), 8.15 (dd, J=8.6, 1.5 Hz, 1H, ArH), 7.93 (d, J=8.6 Hz, 1H, ArH), 7.44 (d, J=9.0 Hz, 2H, ArH), 6.92 (d, J=9.0 Hz, 2H, ArH), 4.33 (d, J=9.3 Hz, CH₂), 4.03 (d, J=9.3 Hz, CH₂), 3.84 (s, 2H, CH₂I), 1.59 (s, 3H, CH₃) | 546.3 | C₂₀H₁₇F₃IN₃O₄,0.7 H₂O: C, 42.90; H, 3.31; N, 7.50 Found: C, 42.82; H, 3.30; N, 7.48. | 78-80 | 76 |
| 23 | 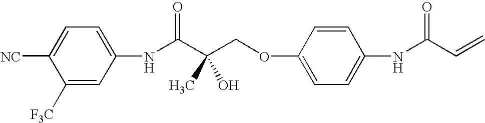 | (CDCl₃) δ 9.21 (bs, 1H, NH), 8.11 (d, J=1.8 Hz, 1H, ArH), 7.96 (dd, J=8.7, 1.8 Hz, 1H, ArH), 7.80 (d, J=8.7 Hz, 1H, ArH), 7.46 (d, J=8.8 Hz, 2H, ArH), 7.38 (bs, 1H, NH), 6.84 (d, J=8.8 Hz, 2H, ArH), 6.44 (d, J=16.8 Hz, 1H, CH═CH₂), 6.24 (dd, J=16.8, 10.2 Hz, 1H, CH═CH₂), 5.78 (d, J=10.2 Hz, 1H, CH═CH₂), 4.42 (d, J=9.0 Hz, 1H, CH₂), 3.97 (d, J=9.0 Hz, 1H, CH₂), 3.77 | | | | |
| # bs, 1H, OH), 1.57 (s, 3H, CH₃) | 432.1 | C₂₁H₁₈F₃N₃O₄,0.3C₄H₃O₂: C, 57.99; H, 4.47; N, 9.14 Found: C, 57.65; H, 4.40; N, 9.15. | 76-78 | 73 | | |
| 14 | 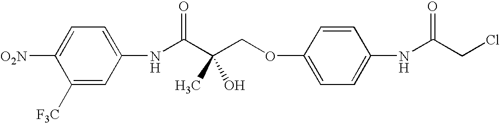 | (DMSO-d₆) δ 10.65 (bs, 1H, NH), 10.16 (bs, 1H, NH), 8.59 (d, J=2.1 Hz, 1H, ArH), 8.39 (dd, J=9.0, 2.1 Hz, 1H, ArH), 8.20 (d, J=9.0 Hz, 1H, ArH), 7.47 (d, J=9.0 Hz, 2H, ArH), 6.90 (d, J=9.0 Hz, 2H, ArH), 6.28 (bs, 1H, OH), 4.22 (d, J=9.0 Hz, CH₂), 4.21 (s, 2H, CH₂Cl), 3.97 (d, J=9.0 Hz, CH₂), 1.45 (s, 3H, CH₃) | 474.0 | C₁₉H₁₇ClF₃N₃O₆, 0.2 C₄H₈ | | |
| # H₈O₂: C, 48.20; H, 3.80; N, 8.52 Found: C, 48.53; H, 3.77; N, 8.59. | 97-99 | 95 | | | | |
| 15 | 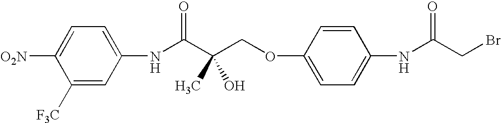 | (DMSO-d₆) δ 10.63 (bs, 1H, NH), 10.23 (bs, 1H, NH), 8.58 (d, J=2.4 Hz, 1H, ArH), 8.38 (dd, J=9.0, 2.4 Hz, 1H, ArH), 8.20 (d, J=9.0 Hz, 1H, ArH), 7.48 (d, J=9.0 Hz, 2H, ArH), 6.90 (d, J=9.0 Hz, 2H, ArH), 6.27 (bs, 1H, OH), 4.20 (d, J=9.8 Hz, CH₂), 3.99 (s, 2H, CH₂Br), 3.98 (d, J=9.8 Hz, CH₂), 1.44 (s, 3H, CH₃) | 518.7 | C₁₉H₁₇BrF₃N₃O₆, 0.5 H₂O.: | | |
| # C, 43.12; H, 3.43; N, 7.94 Found: C, 43.15; H, 3.20; N, 7.73. | 104-106 | 75 | | | | |
| 16 | 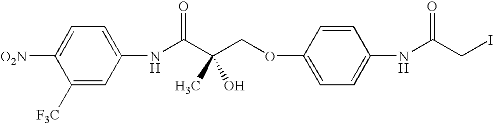 | (CD₃OD) δ 8.38 (d, J=1.8 Hz, 1H, ArH), 8.19 (dd, J=8.9, 1.8 Hz, 1H, ArH), 8.06 (d, J=8.9 Hz, 1H, ArH), 7.45 (d, J=9.0 Hz, 2H, ArH), 6.94 (d, J=9.0 Hz, 2H, ArH), 4.34 (d, J=9.5 Hz, CH₂), 4.04 (d, =9.5 Hz, CH₂), 3.84 (s, 2H, CH₂I), 1.55 (s, 3H, CH₃) | 566.0 | C₁₉H₁₇F₃IN₃O₆, 0.2C₄H₈O₂.: C, 40.66; H, 3.21; N, 7.18 Found: C, 40.87; H, | | |
| # 3.07; N, 7.17. | 82-84 | 76 | | | | |
| 24 | 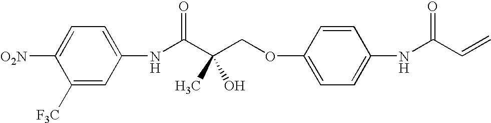 | (CDCl₃) δ 9.37 (bs, 1H, NH), 8.09 (s, 1H, ArH), 7.97 (m, 2H, ArH), 7.72 (bs, 1H, NH), 7.36 (d, J=8.4 Hz, 2H, ArH), 6,73 (d, J=8.4 Hz, 2H, ArH), 6.40 (d, J=16.8 Hz, 1H, CH═CH₂), 6.25 (dd, J=16.8, 10.2 Hz, 1H, CH═CH₂), 5.75 (d, J=10.2 Hz, 1H, CH═CH₂), 4.36 (d, J=9.00 Hz, 1H, CH₂), 4.19 (bs, 1H, OH), 3.91 (d, J=9.00 Hz, 1H, CH₂), 1.63 (s, 3H, CH₃) | 452.1 | | | |
| # C₂₀H₁₈F₃N₂O₆: C, 52.98; H, 4.00; N, 9.27 Found: C, 53.10; H, 4.13; N, 9.03. | 76-78 | 73 | | | | |
| 17 | 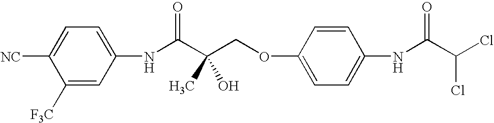 | (DMSO-d₆) δ 10.59 (bs, 1H, NH), 10.50 (bs, 1H, NH), 8.56 (s, 1H, ArH), 8.32 (d, J=5.7 Hz, 1H, ArH), 8.11 (d, J=5.7 Hz, 1H, ArH), 7.49 (d, J=5.4 Hz, 2H, ArH), 6.94 (d, J=5.4 Hz, 2H, ArH), 6.55 (s, 1H, CH), 6.28 (bs, 1H, OH), 4.22 (d, J=7.2 Hz, 1H, CH₂), 3.99 (d, J=7.2 Hz, 1H, CH₂), 1.44 (s, 3H, CH₃) | 488.3 | C₂₀H₁₆Cl₂F₃N₃O₄.0.5C₄H₈O₂: | | |
| # C, 49.24; H, 3.54; N, 8.20 Found: C, 49.21; H, 3.51; N, 8.16. | 140-142 | 72 | | | | |
| 18 | 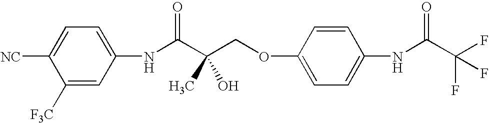 | (DMSO-d₆) δ 11.10 (bs, 1H, NH), 10.59 (bs, 1H, NH), 8.57 (s, 1H, ArH), 8.33 (d, J=8.0 Hz, 1H, ArH), 8.11 (d, J=8.0 Hz, 1H, ArH), 7.56 (d, J=8.1 Hz, 2H, ArH), 6.98 (d, J=8.1 Hz, 2H, ArH), 6.29 (bs, 1H, OH), 4.25 (d, J=9.1 Hz, 1H, CH₂), 4.02 (d, J=9.1 Hz, 1H, CH₂), 1.20 (s, 3H, CH₃) | 474.0 | C₂₀H₁₅F₆N₃O₄.: C, 50.54; H, 3.18; N, 8.84 Found: C, 50.50; H, 3.38; N, 8.67 | | |
| # 80-82 | 79 | | | | | |
| 19 | 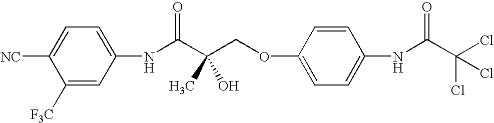 | (DMSO-d₆) δ 10.67 (bs, 1H, NH), 10.60 (bs, 1H, NH), 8.57 (s, 1H, ArH), 8.33 (d, J=8.4 Hz, 1H, ArH), 8.12 (d, J=8.4 Hz, 1H, ArH), 7.52 (d, J=8.7 Hz, 2H, ArH), 6.97 (d, J=8.7 Hz, 2H, ArH), 4.25 (d, J=9.3 Hz, 1H, CH₂), 4.01 (d, J=9.3 Hz, 1H, CH₂), 1.45 (s, 3H, CH₃) | 522.2 | C₂₀H₁₇F₃N₃O₄.0.7H₂O: C, 45.31; H, 2.97; N, 7.93 Found: C, 45.29; H, 2.94; N, 7.68. | | |
| # 153-155 | 75 | | | | | |
| 20 | 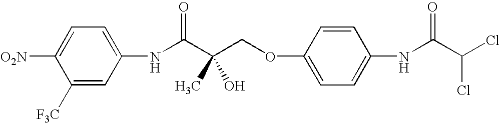 | (CDCl₃) δ 9.30 (bs, 1H, NH), 8.26 (bs, 1H, NH), 8.12 (d, J=1.8 Hz, 1H, ArH), 8.04 (dd, J=9.0, 1.8 Hz, 1H, ArH), 7.98 (d, J=9.0 Hz, 1H, ArH), 7.43 (d, J=9.0 Hz, 2H, ArH), 6.89 (d, J=9.0 Hz, 2H, ArH), 6.08 (s, 1H, CH), 4.46 (d, J=9.0 Hz, CH₂), 3.99 (d, J=9.0 Hz, CH₂), 3.51 (bs, 1H, OH), ), 1.61 (s, 3H, CH₃) | 508.2 | C₁₉H₁₆Cl₂F₃N₃O₆.0.5 H₂O.: C, | | |
| # 43.95; H, 3.30; N, 8.09 Found: C, 43.99; H, 3.19; N, 7.95 | 64-66 | 82 | | | | |
| 21 | 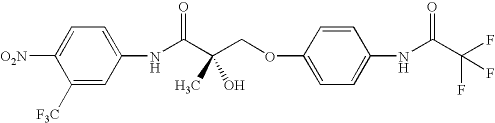 | (CDCl₃) δ 9.23 (bs, 1H, NH), 8.12 (bs, 1H, NH), 8.02 (m, 3H, ArH), 7.49 (d, J=9.0 Hz, 2H, ArH), 6.92 (d, J=9.0 Hz, 2H, ArH), 4.48 (d, J=9.0 Hz, CH₂), 4.01 (d, J=9.0 Hz, CH₂), 3.55 (bs, 1H, OH), 1.62 (s, 3H, CH₃), | 494.0 | C₁₉H₁₅F₆N₃O₆.: C, 46.07; H, 3.05; N, 8.48 Found: C, 45.89; H, 3.16; N, 8.20 | 58-60 | 83 |
| 22 | 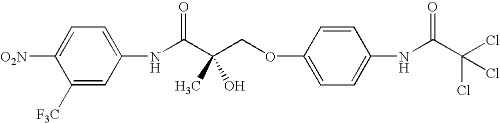 | (CDCl₃) δ 9.28 (bs, 1H, NH), 8.37 (bs, 1H, NH), 8.13 (m, 1H, ArH), 8.03 (m, 2H, ArH), 7.49 (d, J=9.0 Hz, 2H, ArH), 6.94 (d, J=9.0 Hz, 2H, ArH), 4.49 (d, J=9.3 Hz, CH₂), 4.03 (m, 2H, CH₂and OH), 1.62 (s, 3H, CH₃) | 542.2 | C₁₉H₁₅Cl₃F₃N₃O₆.0.5 H₂O: C, 41.21; H, 2.91; N, 7.59 Found: C, 41.07; H, 2.68; N, 7.65 | 59-61 | 79 |
| 25 | 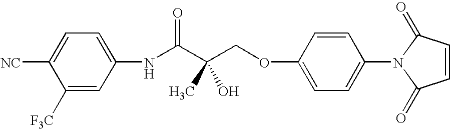 | (CDCl₃) δ 9.18 (bs, 1H, NH), 8.11 (d, J=1.8 Hz 1H, ArH), 7.98 (dd, J=8.4, 1.8 Hz, 1H, ArH), 7.80 (d, J=8.4 Hz, 1H, ArH), 7.26 (d, J=9.0 Hz, 2H, ArH), 6.99 (d, J=9.0 Hz, 2H, ArH), 6.84 (s, 2H, CH═CH), 4.48 (d, J=9.3 Hz, 1H, CH₂), 4.02 (d, J=9.0 Hz, 1H, CH₂), 3.57 (bs, 1H, OH), 1.59 (s, 3H, CH₃) | 458.1 | C₂₂H₁₆F₃N₃O₅: C, 57.52; H, 3.51; N, 9.15. Found: | | |
| # C, 57.72; H, 3.72; N, 8.87. | 144-146 | 60 | | | | |
| 26 | 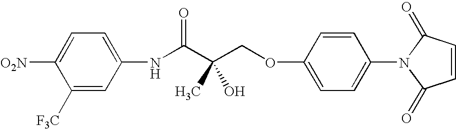 | (CDCl₃) δ 9.26 (bs, 1H, NH), 8.10 (s, 1H, ArH), 8.02 (m, 2H, ArH), 7.25 (d, J=8.7 Hz, 2H, ArH), 6.98 (d, J=8.7 Hz, 2H, ArH), 6.84 (s, 2H, CH═CH), 4.48 (d, J=9.0 Hz, 1H, CH₂), 4.02 (d, J=9.0 Hz, 1H, CH₂), 3.63 (bs, 1H, OH), 1.60 (s, 3H, CH₃) | 477.9 | C₂₁H₁₆F₃N₃O₇.0.5H₂O: C, 51.65; H, 3.51; N, 8.60. Found: C, 51.63; H, 3.44; N, 8.35 | 82-84 | 67 |

It will be appreciated by a person skilled in the art that the present invention is not limited by what has been particularly shown and described hereinabove. Rather, the scope of the invention is defined by the claims that follow:

1. An anti-cancer compound represented by the structure of formula I: 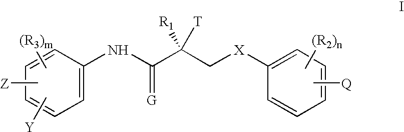
X is a bond, O, CH₂, NH, S, SO, SO₂, Se, PR, NO or NR;
G is O or S;
T is OH, OR, —NHCOCH₃, NHCOR or 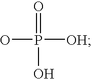
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 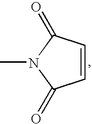
with the provisio that when Z is NO₂, CN, COR, COOH or CONHR, Q is not NHCOCH₂A or N₃;
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 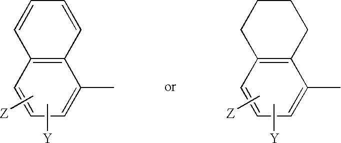
n is an integer of 1-4; and
m is an integer of 1-3.

2. A anti-cancer compound represented by the structure of formula I: 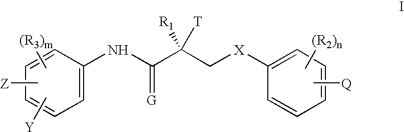
wherein
X is a bond, O, CH₂, NH, S, SO, SO₂, Se, PR, NO or NR;
G is O or S;
T is OH, OR, —NHCOCH₃, NHCOR or 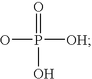
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 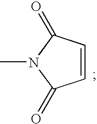
with the provisio that when Z is NO₂, CN, COR, COOH or CONHR, Q is not NHCOCH₂A or N₃;
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃, SnR₃, or R₃ together with the benzene ring to which it is attached forms a fused ring system represented by the structure: 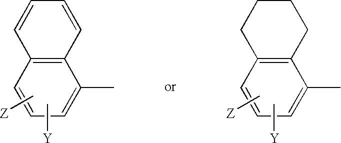
n is an integer of 1-4; and
m is an integer of 1-3;
or its analog, isomer, metabolite, derivative, pharmaceutically acceptable salt, pharmaceutical product, N-oxide, hydrate or any combination thereof.

3. The compound according to claim 1, wherein G is O.

4. The compound according to claim 1, wherein T is OH.

5. The compound according to claim 1, wherein R₁ is CH₃.

6. The compound according to claim 1, wherein X is O.

7. The compound according to claim 1, wherein Z is NO₂.

8. The compound according to claim 1, wherein Z is CN.

9. The compound according to claim 1, wherein Y is CF₃.

10. The compound according to claim 1, wherein Q is NHCOCH₂Cl.

11. The compound according to claim 1, wherein Q is NHCOCH₂Br.

12. The compound according to claim 1, wherein said compound is an alkylating agent.

13. A anti-cancer compound represented by the structure of formula II: 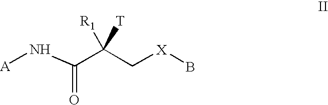
wherein
X is a bond, O, CH₂, NH, S, SO, SO₂, Se, PR, NO or NR;
G is O or S;
T is OH, OR, —NHCOCH₃, NHCOR or 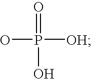
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 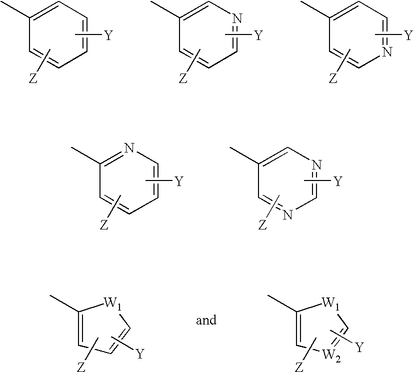
B is a ring selected from: 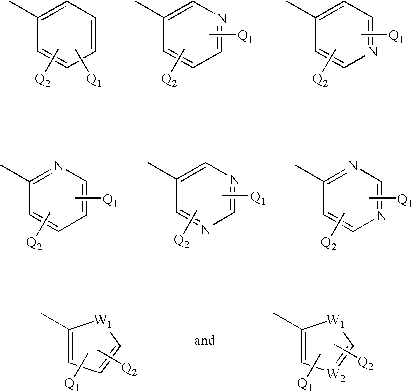
wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 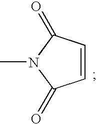
with the provisio that when Z is NO₂, CN, COR, COOH or CONHR, Q is not NHCOCH₂A or N₃;
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 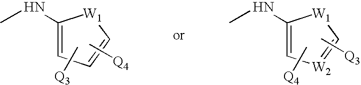
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

14. A anti-cancer compound represented by the structure of formula II: 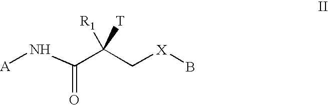
wherein
X is a bond, O, CH₂, NH, S, SO, SO₂, Se, PR, NO or NR;
G is O or S;
T is OH, OR, —NHCOCH₃, NHCOR or 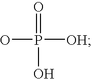
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 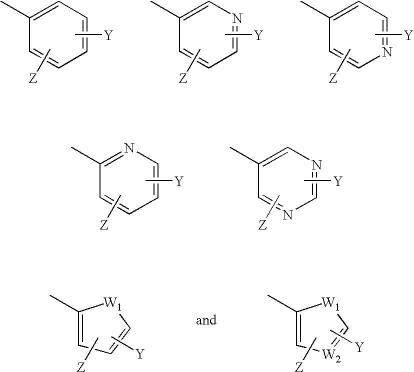
B is a ring selected from: 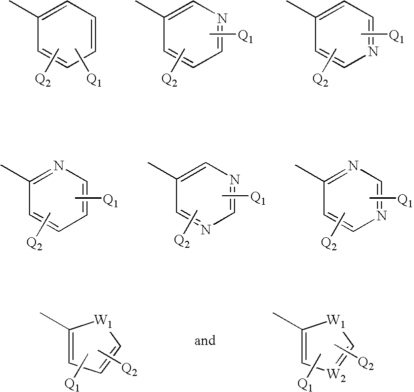
wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 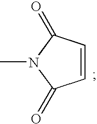
with the provisio that when Z is NO₂, CN, COR, COOH or CONHR, Q is not NHCOCH₂A or N₃;
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 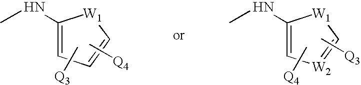
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;
or its analog, isomer, metabolite, derivative, pharmaceutically acceptable salt, pharmaceutical product, N-oxide, hydrate or any combination thereof.

15. The compound according to claim 13, wherein G is O.

16. The compound according to claim 13, wherein T is OH.

17. The compound according to claim 13, wherein R₁ is CH₃.

18. The compound according to claim 13, wherein X is O.

19. The compound according to claim 13, wherein Z is NO₂.

20. The compound according to claim 13, wherein Z is CN.

21. The compound according to claim 13, wherein Y is CF₃.

22. The compound according to claim 13, wherein Q₁ is NHCOCH₂Cl.

23. The compound according to claim 13, wherein Q₁ is NHCOCH₂Br.

24. The compound according to claim 13, wherein said compound is an alkylating agent.

25. A anti-cancer compound represented by the structure of formula III: 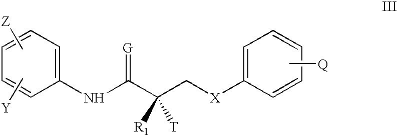
wherein
X is a bond, O, CH₂, NH, S, SO, SO₂, Se, PR, NO or NR;
G is O or S;
T is OH, OR, —NHCOCH₃, NHCOR or 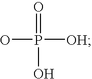
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 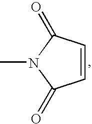
with the provisio that when Z is NO₂, CN, COR, COOH or CONHR, Q is not NHCOCH₂A or N₃;A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

26. A anti-cancer compound represented by the structure of formula III: 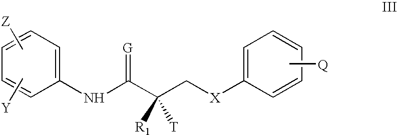
wherein
X is a bond, O, CH₂, NH, S, SO, SO₂, Se, PR, NO or NR;
G is O or S;
T is OH, OR, —NHCOCH₃, NHCOR or 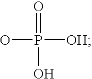
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 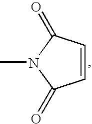
with the provisio that when Z is NO₂, CN, COR, COOH or CONHR, Q is not NHCOCH₂A or N₃;
A is F, C, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃;
or its analog, isomer, metabolite, derivative, pharmaceutically acceptable salt, pharmaceutical product, N-oxide, hydrate or any combination thereof.

27. The compound according to claim 25, wherein G is O.

28. The compound according to claim 25, wherein T is OH.

29. The compound according to claim 25, wherein R₁ is CH₃.

30. The compound according to claim 25, wherein X is O.

31. The compound according to claim 25, wherein Z is NO₂.

32. The compound according to claim 25, wherein Z is CN.

33. The compound according to claim 25, wherein Y is CF₃.

34. The compound according to claim 25, wherein Q is NHCOCH₂Cl.

35. The compound according to claim 25, wherein Q is NHCOCH₂Br.

36. The compound according to claim 25, wherein Q is COCH═CH₂.

37. The compound according to claim 25, wherein Q is COCH₂A.

38. The compound according to claim 25, wherein Q is

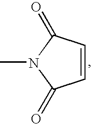

39. The compound according to claim 25, wherein said compound is an alkylating agent.

40. The compound according to claim 25, represented by the structure of formula IV:

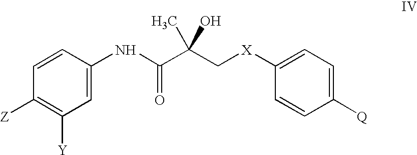

41. A process for preparing an anti-cancer compound represented by the structure of formula I: 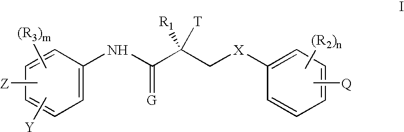
wherein
X is a bond, O, CH₂, NH, S, SO, SO₂, Se, PR, NO or NR;
G is O or S;
T is OH, OR, —NHCOCH₃, NHCOR, or 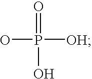
Y is CF₃ F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 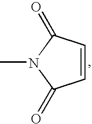
with the provisio that when Z is NO₂, CN, COR, COOH or CONHR, Q is not NHCOCH₂A or N₃;
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 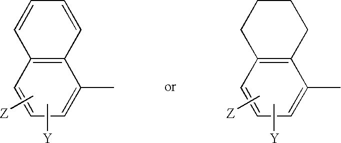
n is an integer of 1-4; and
m is an integer of 1-;
said process comprising the step of coupling a compound of formula VIII: 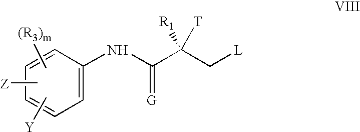
wherein Z, Y, G, R₁, T, R₃ and m are as defined above and L is a leaving group, with a compound of formula IX: 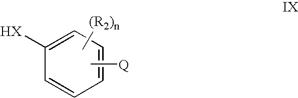
wherein Q, X R₂ and n are as defined above.

42. A process for preparing an anti-cancer compound represented by the structure of formula II: 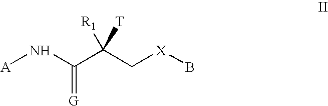
wherein
X is a bond, O, CH₂, NH, S, SO, SO₂, Se, PR, NO or NR;
G is O or S;
T is OH, OR, —NHCOCH₃, NHCOR or 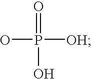
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 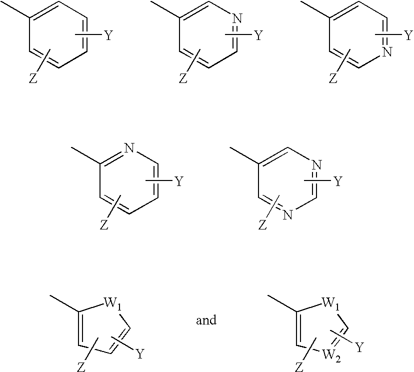
B is a ring selected from: 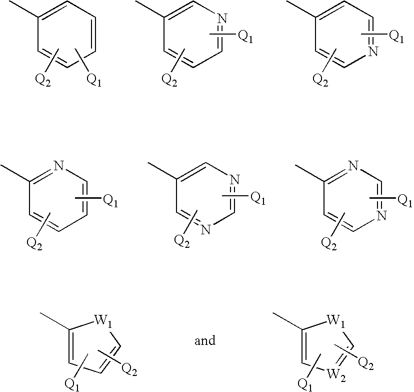
wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 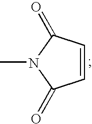
with the provisio that when Z is NO₂, CN, COR, COOH or CONHR, Q is not NHCOCH₂A or N₃;
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 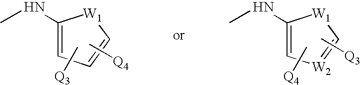
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;
said process comprising the step of coupling a compound of formula XIII: 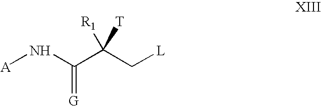
wherein A, G, R₁ and T are as defined above and L is a leaving group, with a compound of formula HX—B wherein B and X are as defined above.

43. A process for preparing an anti-cancer compound represented by the structure of formula III: 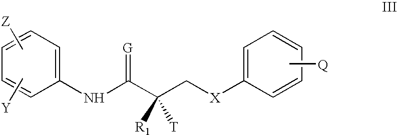
wherein
X is a bond, O, CH₂, NH, S, SO, SO₂, Se, PR, NO or NR;
G is O or S;
T is OH, OR, —NHCOCH₃, NHCOR or 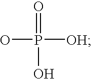
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 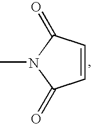
with the provisio that when Z is NO₂, CN, COR, COOH or CONHR, Q is not NHCOCH₂A or N₃;
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃ said process comprising the step of coupling a compound of formula XIV: 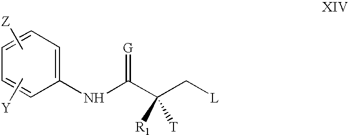
wherein Z, Y, G R₁ and T are as defined above and L is a leaving group, with a compound of formula XV: 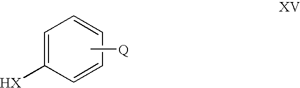
wherein Q and X are as defined above.

44. The process according to claim 43, wherein said anti-cancer compound is represented by the structure of formula IV:

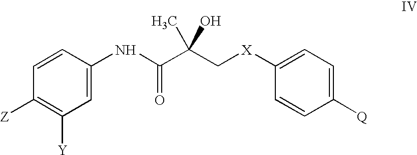

45. An anti-cancer compound represented by the structure of formula I: 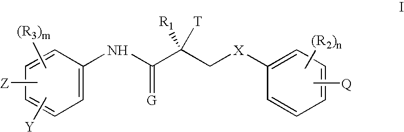
wherein X is SO or SO₂;
G is O or S;
T is OH, OR, —NHCOCH₃, NHCOR or 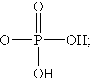
Y is CF₃ F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 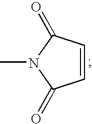
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 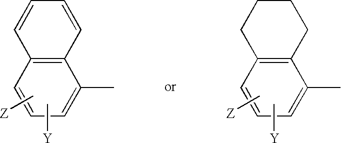
n is an integer of 1-4; and
m is an integer of 1-3.

46. A anti-cancer compound represented by the structure of formula I: 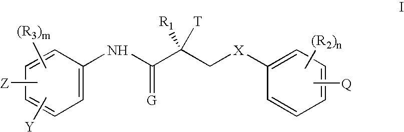
wherein
X is SO or SO₂;
G is O or S;
T is OH, OR, —NHCOCH₃, NHCOR or 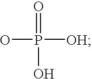
Y is CF₃, F, Cl, Br, I, CN, or SnR3;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH2A or 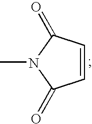
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃, SnR₃, or R₃ together with the benzene ring to which it is attached forms a fused ring system represented by the structure: 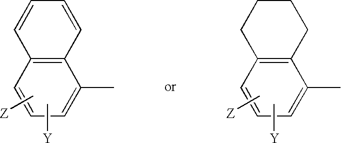
n is an integer of 1-4; and
m is an integer of 1-3;
or its analog, isomer, metabolite, derivative, pharmaceutically acceptable salt, pharmaceutical product, N-oxide, hydrate or any combination thereof.

47. The compound according to claim 45, wherein G is O.

48. The compound according to claim 45, wherein T is OH.

49. The compound according to claim 45, wherein R₁ is CH₃.

50. The compound according to claim 45, wherein Z is NO₂.

51. The compound according to claim 45, wherein Z is CN.

52. The compound according to claim 45, wherein Y is CF₃.

53. The compound according to claim 45, wherein Q is NHCOCH₂Cl.

54. The compound according to claim 45, wherein Q is NHCOCH₂Br.

55. The compound according to claim 45, wherein said compound is an alkylating agent.

56. A anti-cancer compound represented by the structure of formula II: 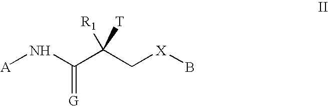
wherein
X is SO or SO₂;
G is O or S;
T is OH, OR, —NHCOCH₃, NHCOR or 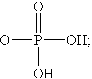
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 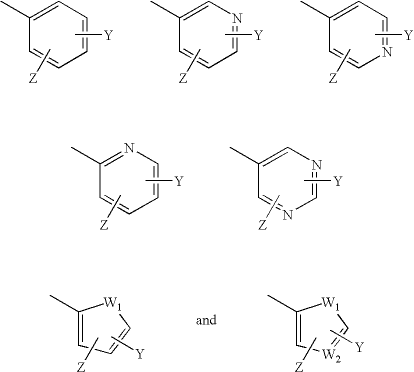
B is a ring selected from: 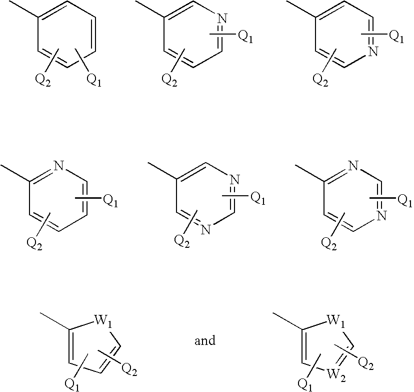
wherein A and B cannot simultaneously be a benzene ring;
Y is CF₃, F, I, Br, Cl, CN CR₃ or SnR₃;
one of Z or Q, is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂ COCH═CH₂, COCH₂A or 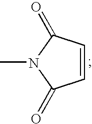
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 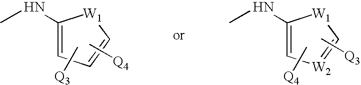
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

57. A anti-cancer compound represented by the structure of formula II: 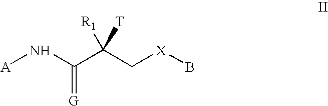
wherein
X is SO or SO₂;
G is O or S;
T is OH, OR, —NHCOCH₃, NHCOR or 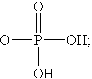
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 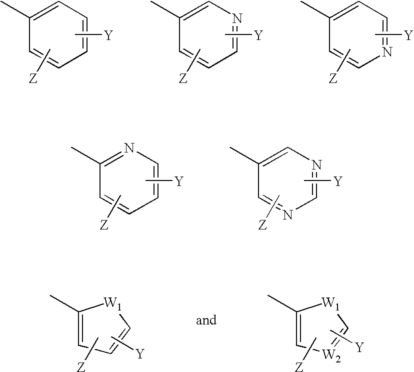
B is a ring selected from: 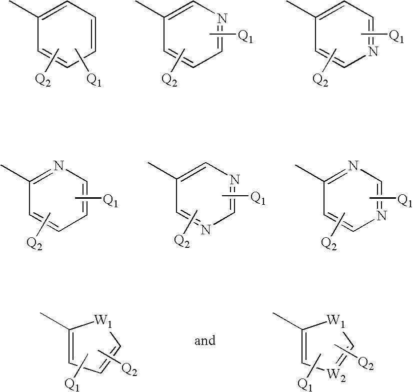
wherein A and B cannot simultaneously be a benzene ring;
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 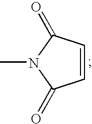
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 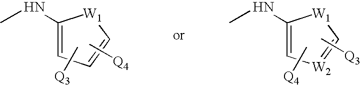
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;
or its analog, isomer, metabolite, derivative, pharmaceutically acceptable salt, pharmaceutical product, N-oxide, hydrate or any combination thereof.

58. The compound according to claim 56, wherein G is O.

59. The compound according to claim 56, wherein T is OH.

60. The compound according to claim 56, wherein R₁ is CH₃.

61. The compound according to claim 56, wherein Z is NO₂.

62. The compound according to claim 56, wherein Z is CN.

63. The compound according to claim 56, wherein Y is CF₃.

64. The compound according to claim 56, wherein Q₁ is NHCOCH₂Cl.

65. The compound according to claim 56, wherein Q₁ is NHCOCH₂Br.

66. The compound according to claim 56, wherein said compound is an alkylating agent.

67. A anti-cancer compound represented by the structure of formula III: 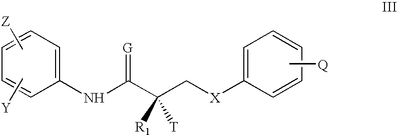
wherein
X is SO or SO₂;
G is O or S;
T is OH, OR, —NHCOCH₃, NHCOR or 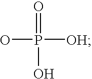
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 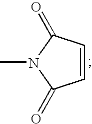
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

68. A anti-cancer compound represented by the structure of formula III: 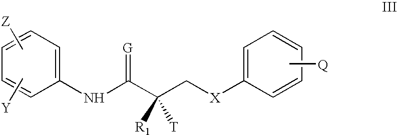
wherein
X SO or SO₂;
G is O or S;
T is OH, OR, —NHCOCH₃, NHCOR or 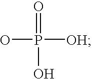
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 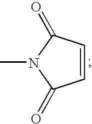
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃;
or its analog, isomer, metabolite, derivative, pharmaceutically acceptable salt, pharmaceutical product, N-oxide, hydrate or any combination thereof.

69. The compound according to claim 67, wherein G is O.

70. The compound according to claim 67, wherein T is OH.

71. The compound according to claim 67, wherein R₁ is CH₃.

72. The compound according to claim 67, wherein Z is NO₂.

73. The compound according to claim 67, wherein Z is CN.

74. The compound according to claim 67, wherein Y is CF₃.

75. The compound according to claim 67, wherein Q is NHCOCH₂Cl.

76. The compound according to claim 67, wherein Q is NHCOCH₂Br.

77. The compound according to claim 67, wherein said compound is an alkylating agent.

78. The compound according to claim 67, represented by the structure of formula IV:

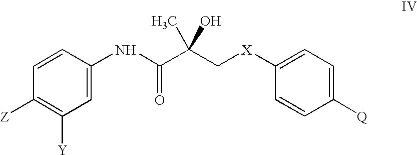

79. A process for preparing an anti-cancer compound represented by the structure of formula I: 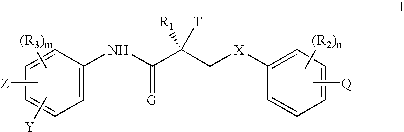
wherein
X is SO or SO₂;
G is O or S;
T is OH, OR, —NHCOCH₃, NHCOR, or 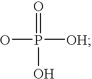
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 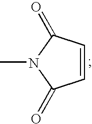
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
each of R₂, independently, are F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
each of R₃, independently, are F, Cl, Br, I, CN, NO₂, COR, COOH, CONHR, CF₃ or SnR₃, or R₃ together with the benzene ring to which it is attached forms a fused ring system represented by the structure: 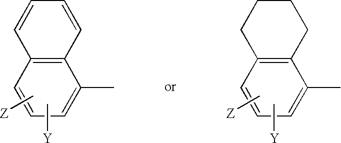
n is an integer of 1-4; and
m is an integer of 1-;
said process comprising the step of coupling a compound of formula VIII: 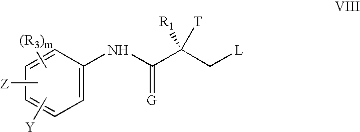
wherein Z, Y, G, R₁, T, R₃ and m are as defined above and L is a leaving group, with a compound of formula IX: 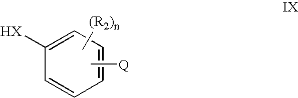
wherein Q, X, R₂ and n are as defined above.

80. A process for preparing an anti-cancer compound represented by the structure of formula II: 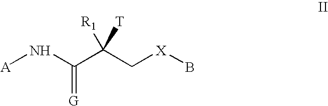
wherein
X is SO or SO₂;
G is O or S;
T is OH, OR, —NHCOCH₃, NHCOR or 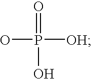
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 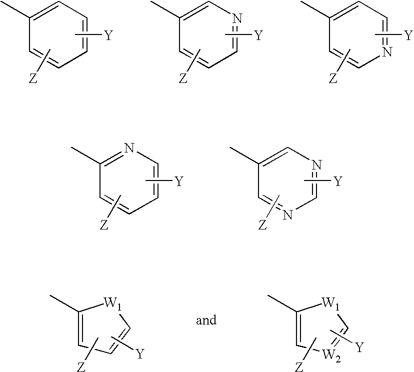
B is a ring selected from: 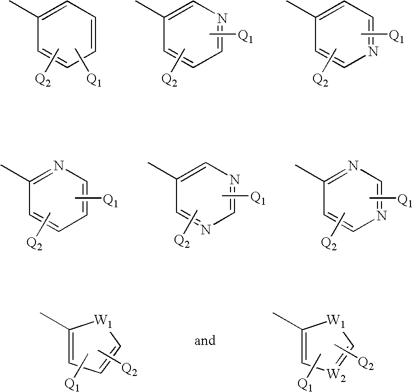
Y is CF₃, F, I, Br, Cl, CN CR₃ or SnR₃;
one of Z or Q₁ is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 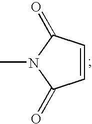
A is F, Cl, Br or I
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 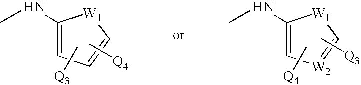
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;
said process comprising the step of coupling a compound of formula XIII: 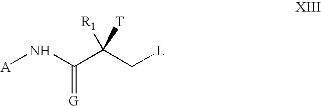
wherein A, G, R₁ and T are as defined above and L is a leaving group, with a compound of formula HX—B wherein B and X are as defined above.

81. A process for preparing an anti-cancer compound represented by the structure of formula III: 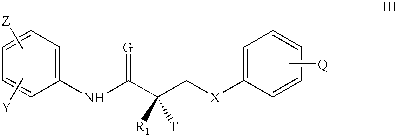
wherein
X is SO or SO₂;
G is O or S;
T is OH, OR, —NHCOCH₃, NHCOR or 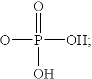
Y is CF₃, F, Cl, Br, I, CN, or SnR₃;
one of Z or Q is NO₂, CN, COR, COOH, CONHR, F, Cl, Br or I, and the other is NCS, NHCOCH₂A, N₃, SO₂F, N(OH)COR, CONHOH, NHSO₂CH₂A, NHCOCH═CH₂, COCH═CH₂, COCH₂A or 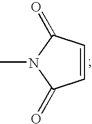
A is F, Cl, Br or I;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃ said process comprising the step of coupling a compound of formula XIV: 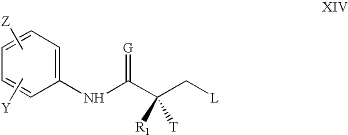
wherein Z, Y, G R₁ and T are as defined above and L is a leaving group, with a compound of formula XV: 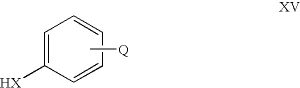
wherein Q and X are as defined above.

82. The process according to claim 81, wherein said anti-cancer compound is represented by the structure of formula IV:

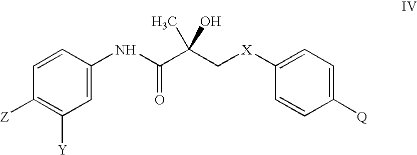

83. An anti-cancer compound represented by the structure of formula XIX: 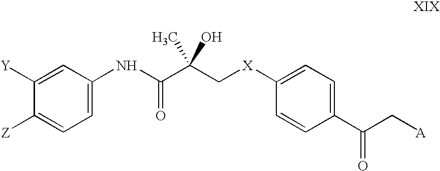
wherein
X is a bond, O, CH₂, NH, S, SO, SO₂, Se, PR, NO or NR;
Y is CF₃, F, Cl, Br, I, CN, or SnR₃;
Z is NO₂, CN, COR, COOH, CONHR, F, Cl, Br or I; and
A is F, Cl, Br or I.

84. An anti-cancer compound represented by the structure of formula XX: 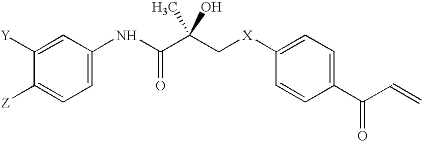
wherein
X is a bond, O, CH₂, NH, S, SO, SO₂, Se, PR, NO or NR;
Y is CF₃, F, Cl, Br, I, CN, or SnR₃; and
Z is NO₂, CN, COR, COOH, CONHR, F, Cl, Br or I.

85. An anti-cancer compound represented by the structure of formula XXI: 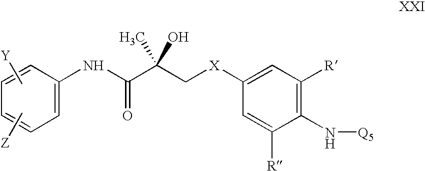
wherein
X is O, S, or SO₂;
Y is CF₃, F, Cl, Br, I, CN, or SnR₃;
Z is NO₂, CN, COR, COOH, CON, F, Cl, Br or I;
Q₅ is COCH═CH₂ or COCH₂A;
A is F, Cl, Br or I; and
each of R′ and R″, independently, are H, F, Cl, Br, I or alkyl.

86. The compound according to claim 85, represented by the structure of formula XXII: 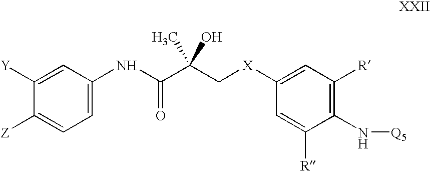
wherein X, Y, Z, Q₅, R′ and R″ are as defined in claim 85.

87. An anti-cancer compound represented by the structure of the formula XXIII: 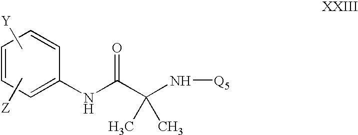
wherein
Y is CF₃, F, Cl, Br, I, CN, or SnR₃;
Z is NO₂, CN, COR, COOH, CONHR, F, Cl, Br or I;
Q₅ is COCH═CH₂ or COCH₂A; and
A is F, Cl, Br or I.

88. A compound according to claim 87, represented by the structure of the formula XXIV: 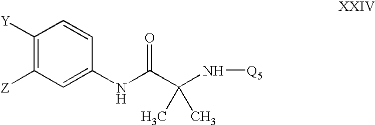
wherein Y, Z and Q₅ are as defined in claim 87.

89. An anti-cancer compound represented by the structure of the formula XXIV: 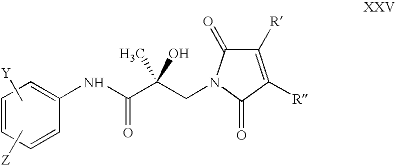
wherein
Y is CF₃, F, Cl, Br, I, CN, or SnR₃;
Z is NO₂, CN, COR, COOH, CONHR, F, Cl, Br or I; and
R′ and R″ are, independently of each other, a hydrogen, alkyl or a halogen.

90. A compound according to claim 89, represented by the structure of the formula XXVI: 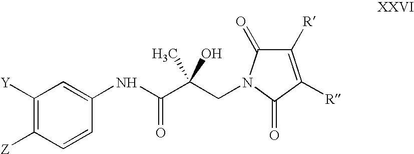
wherein Y, Z, R′ and R″ are as defined in claim 89.

91. A process for preparing an anti-cancer compound represented by the structure of the formula XXI: 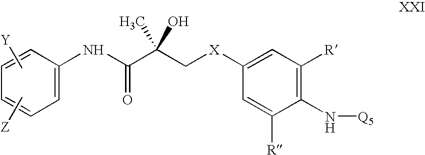
wherein
X is O, S, or SO;
Y is CF₃, F, Cl, Br, I, CN, or SnR₃;
Z is NO₂, CN, COR, COOH, CONHR, F, Cl, Br or I;
Q₅ is COCH═CH₂ or COCH₂A;
A is F, Cl, Br or I; and
each of R′ and R″, independently, are H, F, Cl, Br, I or alkyl. the process comprising the steps of:
coupling a compound represented by the structure of the formula XXVII: 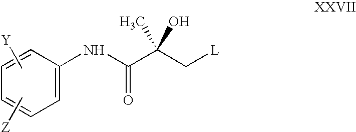
wherein Y and Z are defined above and L is a leaving group, with a compound represented by the structure of the formula XXVIII: 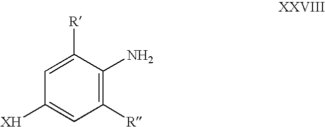
wherein R′, R″ and X are defined above, to prapare a compound represented by the structure of the formula XXIX: 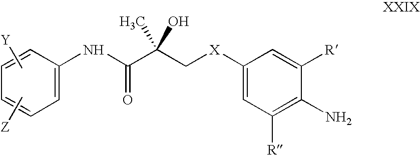
wherein R′, R″, X, Y and Z are defined above; and reacting the compound formula XXIX with A-Q₅, wherein A and Q₅ are defined above, to obtain a compound represented by the structure of the formula XXI.

92. A process for preparing an anti-cancer compound represented by the structure of the formula XXIII: 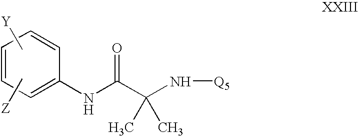
wherein
Y is CF₃, F, Cl, Br, I, CN, or SnR₃;
Z is NO₂, CN, COR, COOH, CONHR, F, Cl, Br or I;
Q₅ is COCH═CH₂ or COCH₂A; and
A is F, Cl, Br or I,
the process comprising the step of:
reacting a compound represented by the structure of the formula XXX: 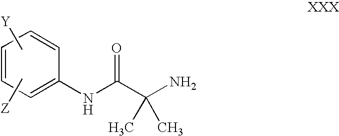
wherein Y and Z are defined above,
with A-Q₅, wherein A and Q₅ are defined above, to obtain a compound represented by the structure of the formula XXIII.

93. A process for preparing an anti-cancer compound represented by the structure of the formula XXV: 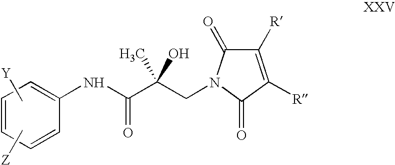
wherein
Y is CF₃, F, Cl, Br, I, CN, or SnR₃;
Z is NO₂, CN, COR, COOH, CONHR, F, Cl, Br or I; and
R′ and R″ are, independently of each other, a hydrogen, alkyl or a halogen,
the process comprising the step of:
reacting a compound represented by the structure of the formula XXXI: 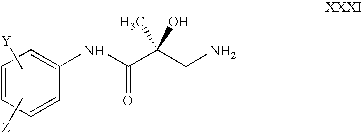
wherein Y and Z are defined above, with a an unsubstituted or a substituted phthalic anhydride represented by the structure of the formula XXXII: 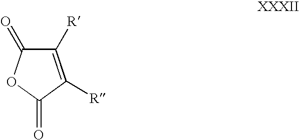
wherein R′ and R″ are defined above, to obtain a compound represented by the structure of the formula XXV.

94. A composition comprising the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof; and a suitable carrier or diluent.

95. A pharmaceutical composition comprising an effective amount of the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof; and a pharmaceutically acceptable carrier, diluent or salt.

96. A method of treating cancer in a subject in need thereof, comprising the step of administering to said subject the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to treat cancer in said subject.

97. A method of preventing cancer in a subject, comprising the step of administering to said subject the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to prevent cancer in said subject.

98. A method of delaying the progression of cancer in a subject in need thereof, comprising the step of administering to said subject the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to delay the progression of cancer in said subject.

99. A method of treating the recurrence of cancer in a subject in need thereof, comprising the step of administering to said subject the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to treat the recurrence of cancer in said subject.

100. A method of preventing the recurrence of cancer in a subject, comprising the step of administering to said subject the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to prevent the recurrence of cancer in said subject.

101. A method of suppressing, inhibiting or reducing the incidence of cancer in a subject in need thereof, comprising the step of administering to said subject the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to suppress, inhibit or reduce the incidence of cancer in said subject.

102. A method of inducing apoptosis in a cancer cell, the step of administering to said subject the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to induce apoptosis in said cancer cell.

103. A method of alkylating a cellular component, comprising the step of contacting a cell comprising said cellular component with the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to alkylate said cellular component.

104. A method of irreversibly binding an anti-cancer compound to a cellular component, comprising the step of contacting a cell comprising said cellular component with the anti-cancer compound of claim 1 and/or its analog, derivative, isomer, metabolite, pharmaceutically acceptable salt, pharmaceutical product, hydrate or N-oxide, impurity, prodrug, polymorph, crystal, or any combination thereof, in an amount effective to irreversibly bind the anti-cancer compound to said cellular component.
